(19) United States
(12) Patent Application Publication
(10) Pub. No.: US 2025/0151731 A1
(43) Pub. Date: May 15, 2025

(54) METHYLOBACTERIUM COMPOSITIONS FOR FUNGAL DISEASE CONTROL

(71) Applicant: NewLeaf Symbiotics, Inc., St. Louis, MO (US)

(72) Inventors: Renèe A. Rioux, Morrisville, NC (US); Charles Michael McFatrich, Washington, MO (US); Kishore Nannapaneni, St. Louis, MO (US)

(21) Appl. No.: 19/028,337

(22) Filed: Jan. 17, 2025

Related U.S. Application Data
(63) Continuation of application No. 18/583,042, filed on Feb. 21, 2024.
Application No. 18/583,042 is a continuation of application No. 17/686,783, filed on Mar. 4, 2022, now Pat. No. 11,930,822.
Application No. 17/686,783 is a continuation of application No. 16/467,384, filed on Jun. 6, 2019, now Pat. No. 11,284,622.
(60) Provisional application No. 62/431,927, filed on Dec. 9, 2016.

(51) Int. Cl. A01N 63/20; A01N 25/04; A01N 33/24; A01N 37/18; A01N 43/54; A01N 43/56; A01N 43/78; A01N 43/80; A01N 43/88

(52) U.S. Cl. CPC A01N 63/20; A01N 25/04; A01N 33/24; A01N 37/18; A01N 43/54; A01N 43/56; A01N 43/78; A01N 43/80; A01N 43/88

(57) ABSTRACT

Compositions comprising Methylobacterium with anti-fungal activity, methods for controlling plant pathogenic fungi, and methods of making the compositions are provided.

### CROSS-REFERENCE TO RELATED APPLICATIONS

This application is a continuation of U.S. patent application Ser. No. 18/538,042 filed Feb. 21, 2024, which is a continuation of U.S. patent application Ser. No. 17/686,783 filed Mar. 4, 2022 (issued as U.S. Pat. No. 11,930,822), which is a continuation of U.S. patent application Ser. No. 16/467,384, filed Jun. 6, 2019 (issued U.S. Pat. No. 11,284,622), which is the 371 National Phase of International Patent Application PCT/US2017/065081, filed Dec. 7, 2017, which claims the benefit of U.S. Provisional Patent Application No. 62/431,927, filed Dec. 9, 2016, the contents of which are incorporated herein by reference in their entirety.

### SEQUENCE LISTING STATEMENT

A computer readable form of the Sequence Listing XML containing the file named “NLSYM9004.USC3 Sequence Listing.xml,” which is 69, 163 bytes in size (as measured in MICROSOFT WINDOWS® EXPLORER) and was created on Jan. 17, 2025, is provided herein and is herein incorporated by reference. This Sequence Listing consists of SEQ ID NOs: 1-60.

### BACKGROUND

One-carbon organic compounds such as methane and methanol are found extensively in nature, and are utilized as carbon sources by bacteria classified as methanotrophs and methylotrophs. Methanotrophic bacteria include species in the genera Methylobacter, Methylomonas, Methylomicrobium, Methylococcus, Methylosinus, Methylocystis, Methylosphaera, Methylocaldum, and Methylocella (Lidstrom, 2006). Methanotrophs possess the enzyme methane monooxygenase that incorporates an atom of oxygen from O₂ into methane, forming methanol. All methanotrophs are obligate one-carbon utilizers that are unable to use compounds containing carbon-carbon bonds. Methylotrophs, on the other hand, can also utilize more complex organic compounds, such as organic acids, higher alcohols, sugars, and the like. Thus, methylotrophic bacteria are facultative methylotrophs. Methylotrophic bacteria include species in the genera Methylobacterium, Hyphomicrobium, Methylophilus, Methylobacillus, Methylophaga, Aminobacter, Methylorhabdus, Methylopila, Methylosulfonomonas, Marinosulfonomonas, Paracoccus, Xanthobacter, Ancylobacter (also known as Microcyclus), Thiobacillus, Rhodopseudomonas, Rhodobacter, Acetobacter, Bacillus, Mycobacterium, Arthobacter, and Nocardia (Lidstrom, 2006).

Most methylotrophic bacteria of the genus Methylobacterium are pink-pigmented. They are conventionally referred to as PPFM bacteria, being pink-pigmented facultative methylotrophs. Green (2005, 2006) identified twelve validated species in the genus Methylobacterium, specifically M. aminovorans, M. chloromethanicum, M. dichloromethanicum, M. extorquens, M. fujisawaense, M. mesophilicum, M. organophilum, M. radiotolerans, M. rhodesianum, M. rhodinum, M. thiocyanatum, and M. zatmanii. However, M. nidulans is a nitrogen-fixing Methylobacterium that is not a PPFM (Sy et al., 2001). Methylobacterium are ubiquitous in nature, being found in soil, dust, fresh water, sediments, and leaf surfaces, as well as in industrial and clinical environments (Green, 2006).

Biotic pressures—the combined effects of pathogens, animals, insects, and weeds—result in an annual loss of 20-40% crop productivity. Additional costs of pest control, reduced crop quality, and funding research to combat these pressures, among other factors, make it difficult to precisely quantify losses due to crop pests and diseases (Savary et al. 2012, Food Sec. DOI: 10.1007/s12571-012-0200-5). Agricultural biologicals present a novel, exciting, and rapidly emerging option to combat crop pests and diseases in a manner that complements and preserves current crop germplasm resistance traits, chemical control options, and cultural practices (Schlaeppi and Bulgarelli 2015, MPMI 28(3): 212-217. DOI: 10.1094/MPMI-10-14-0334-FI).

Fungal diseases are a major cause of yield and quality loss to agronomically important row crops, including corn, soybeans, wheat, cotton, and canola. Among these diseases, species of ascomycete fungi in the genera Cercospora, Colletotrichum, Fusarium, and Septoria are particularly widespread and contribute to significant crop losses. Fusarium species alone caused an estimated 36.2 million bushels/year loss in soybeans from the years 1994-2010 (Wrather et al. 2010). These species cause a range of foliar, seedling, and stalk maladies, and, unlike rusts or other diseases caused by obligate biotrophic pathogens, there is no complete genetic resistance to these diseases. Further, chemical controls often must be applied at very precise times to successfully stymie disease progress while maximizing profits and protecting plant health. The narrow thresholds and windows for control can result in incomplete or failed crop protection. Microbial inoculants, which can colonize from the seed and persist on plants and in soil for the full duration of the growing season, offer extended windows of protection, can compensate for chemical control insufficiencies, and extend the benefits of fungicide through resistance management.

### SUMMARY

Provided herein are compositions comprising Methylobacterium that inhibit growth of a plant pathogenic fungus, methods of using the compositions to control fungal infections of plants, plant parts, and plants derived therefrom, and methods of making the compositions. Such Methylobacterium that inhibit growth of a plant pathogenic fungus are in certain instances referred to herein as “Methylobacterium that inhibit plant pathogenic fungi” or, in certain contexts, as simply “Methylobacterium”. In certain embodiments, Methylobacterium that inhibit growth of a plant pathogenic fungus can be distinguished from other Methylobacterium that do not inhibit plant pathogenic fungi by assaying for the ability of the Methylobacterium to inhibit fungal disease in a plant or on an isolated plant part.

Provided herein are compositions comprising a mono-or co-culture of Methylobacterium that inhibit growth of a plant pathogenic fungus and an agriculturally acceptable excipient and/or an agriculturally acceptable adjuvant. In certain embodiments, the Methylobacterium has at least one gene that encodes a 16S RNA having at least 97%, 98%, 99%, 99.5%, or 100% sequence identity to SEQ ID NO: 8; or the Methylobacterium is NLS0109 or a derivative thereof. In certain embodiments, the Methylobacterium has in its genome one or more polynucleotide marker fragments of at least 50, 60, 100, 120, 180, 200, 240, or 300 nucleotides of SEQ ID NOS: 9-11. In certain embodiments, the Methylobacterium has in its genome one or more marker fragments comprising a sequence having at least 98%, 99%, or 99.5% sequence identity across the entire length of SEQ ID NOS: 9-11. In certain embodiments, the Methylobacterium is not M. radiotolerans or M. oryzae. In certain embodiments, the plant pathogenic fungus is selected from the group consisting of a Blumeria sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyriculariasp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. and a Verticillium sp. In certain embodiments, the Fusarium sp. is selected from the group consisting of Fusarium graminearum, Fusarium verticillioides, Fusarium oxysporum, Fusarium virguliforme, and Fusarium solani. In certain embodiments of any of the aforementioned compositions, the composition comprises a solid substance wherein a mono-culture or co-culture of Methylobacterium is adhered thereto. In certain embodiments, the plant pathogenic fungus is a Rhizoctonia sp. or a Sclerotinia sp. In certain embodiments, the Rhizoctonia sp. is Rhizoctonia solani or Rhizoctonia cerealis. In certain embodiments, the Sclerotinia sp. is Sclerotinia sclerotiorum or Sclerotinia homoeocarpa. In certain embodiments, the composition comprises a colloid formed by the solid substance wherein a mono-culture or co-culture of Methylobacterium is adhered thereto and a liquid. In certain embodiments, the colloid is a gel. In certain embodiments of any of the aforementioned compositions, the composition is an emulsion. In certain embodiments of any of the aforementioned compositions, the Methylobacterium is NLS0109, a derivative thereof, or a Methylobacterium related to NLS0109. In certain embodiments of any of the aforementioned compositions, the composition further comprises a second Methylobacterium strain selected from NLS0017, NLS0020, NLS0021, NLS0042, NLS0064, NLS0066, NLS0089, a derivative thereof, or a Methylobacterium sp. related thereto. In certain embodiments, derivatives of Methylobacterium strains NLS0017, NLS0020, NLS0089 and NL0109 or a Methylobacterium sp. related thereto can be identified in by the presence of one or more marker fragments selected from the group consisting of SEQ ID NO: 9-11, 21-24, 37-39, or 49-51, and can be distinguished from other related Methylobacterium by PCR analysis using specific DNA detection assays that include, but are not limited to, DNA primer and probe combinations provided herein. Use of any of the aforementioned compositions for coating or partially coating a plant part (e.g., a seed) to inhibit growth of any of the aforementioned plant pathogenic fungi is also provided herein.

In certain embodiments of any of the aforementioned compositions, the Methylobacterium is NLS0109 or a derivative thereof. In certain embodiments, the Methylobacterium is NLS0109, a derivative thereof, or a Methylobacterium related to NLS0109 and the plant pathogenic fungus is a Rhizoctonia sp. or a Sclerotinia sp. In certain embodiments, the Methylobacterium is NLS0109, a derivative thereof, or a Methylobacterium related to NLS0109 and the plant pathogenic fungus is Fusarium graminearum, Cercospora zeae-maydis, or Colletotrichum graminicola. In certain embodiments, the Methylobacterium is NLS0109, a derivative thereof, or a Methylobacterium related to NLS0109 and the plant pathogenic fungus is Septoria tritici, Stagonospora nodorum, Pythium spp., Rhizoctonia solani, a Fusarium spp., Magnaportha grisea, Pyrenophora tritici-repentis, Microdochium nivale, Sclerotinia sclerotiorum, Cercospora sojina, Cercospora kikuchii, Fusarium spp., Rhizoctonia solani, Fusarium virguliforme, Pythium spp., Rhizoctonia solani, Gibberella zeae, or a Pythium spp. In any of the aforementioned embodiments, the plant pathogenic fungus that is inhibited can be in its anamorphic form, its teleomorphic form, or in both its anamorphic form and its teleomorphic forms. In any of the aforementioned embodiments, the composition can comprise a fungal inhibitory concentration of the mono-or co-culture of Methylobacterium. Use of any of the aforementioned compositions for coating or partially coating a plant part (e.g., a seed) to inhibit growth of any of the aforementioned plant pathogenic fungi is also provided herein.

In certain embodiments, the Methylobacterium is NLS0109, a derivative thereof, or a Methylobacterium related to NLS0109 and the plant pathogenic fungus is a Rhizoctonia sp. or a Fusarium sp. and the plant is corn or soybean. In certain embodiments of the methods, the Fusarium sp. is one or more species selected from Fusarium graminearum, Fusarium verticillioides, Fusarium oxysporum, Fusarium virguliforme, and Fusarium solani, and the Rhizoctonia is Rhizoctonia solani. In some embodiments, Methylobacterium sp. control of fungal infections by Fusarium and Rhizoctonia is evidenced by improved germination and emergence of seed following application of the Methylobacterium. In certain embodiments, the Methylobacterium is NLS0109, a derivative thereof, or a Methylobacterium related to NLS0109 and the plant pathogenic fungus is a Pythium sp. and the plant is corn or soybean. In certain embodiments of the methods, the Pythium sp. is one or more species selected from Pythium torulosum, Pythium oopapillum, Pythium sylvaticum and Pythium lutarium.

In any of the aforementioned embodiments, the composition can further comprise an antifungal compound. In certain embodiments, the antifungal compound can be an azole, a dithiocarbamate, a strobilurin, a phenylamide, a thiazole-carboxamide, a piperidinyl thiazole isoxazoline or a benzimidazole. In certain embodiments, the azole is ipconazole, tebuconazole, triticonazole, metconazole, prothioconazole, triadimenfon, propiconazole and imazalil. In certain embodiments, the composition further comprises a strobilurin fungicide. In certain embodiments, the strobilurin fungicide is selected from the group consisting of azoxystrobin, pyraclostrobin, fluoxastrobin, and trifloxystrobin. In certain embodiments, the strobilurin fungicide is pyraclostrobin. Use of any of the aforementioned compositions for coating or partially coating a plant part (e.g., a seed) to inhibit growth of any of the aforementioned plant pathogenic fungi is also provided herein. In certain embodiments of any of the aforementioned compositions or uses, the combination of Methylobacterium, crop, pathogen, disease, and/or antifungal compounds is as set forth in Table 4.

Also provided are plants or plant parts that are at least partially coated with any of the aforementioned compositions comprising a mono-or co-culture of Methylobacterium. In certain embodiments, the Methylobacterium has at least one gene that encodes a 16S RNA having at least 97%, 98%, 99%, 99.5%, or 100% sequence identity to SEQ ID NO: 8; or the Methylobacterium is NLS0109 or a derivative thereof. In certain embodiments, the Methylobacterium has in its genome one or more polynucleotide marker fragments of at least 50, 60, 100, 120, 180, 200, 240, or 300 nucleotides of SEQ ID NOS: 9-11. In certain embodiments, the Methylobacterium has in its genome one or more marker fragments comprising a sequence having at least 98%, 99%, or 99.5% sequence identity across the entire length of SEQ ID NOS: 9-11. In certain embodiments, the at least partially coated plant or plant part is a cereal plant or cereal plant part. In certain embodiments, the at least partially coated cereal plant is selected from the group consisting of a rice, wheat, corn, barley, millet, sorghum, oat, and rye plant. In certain embodiments, the at least partially coated cereal plant part is selected from the group consisting of a rice, wheat, corn, barley, millet, sorghum, oat, and rye plant part. In certain embodiments, the at least partially coated plant or plant part is a dicot plant part. In certain embodiments, the dicot plant or plant part is a soybean, peanut, or tomato plant part. In certain embodiments of any of the aforementioned plants or plant parts, the Methylobacterium in the composition was obtained from a plant genus, plant species, plant sub-species, or plant cultivar that is distinct from the genus, species, sub-species, or cultivar of the plant or plant part that is coated with the composition. Also provided are processed plant products that comprise a detectable amount of any of the Methylobacterium of any of the aforementioned compositions.

Also provided herein are methods of identifying compositions including, but not limited to, soil samples, plant parts, including plant seeds, or processed plant products, comprising or coated with Methylobacterium sp. NLS0109, NL0020, NLS0017 or NLS0089 by assaying for the presence of nucleic acid fragments comprising at least 40, 50, 60, 100, 120, 180, 200, 240, or 300 nucleotides of SEQ ID NO: 9-11, 21-24, 37-39, or 49-51 in the compositions. In certain embodiments, such methods can comprise subjecting a sample suspected of containing Methylobacterium sp. NLS0109, NL0020, NLS0017 or NLS0089 to a nucleic acid analysis technique and determining that the sample contains one or more nucleic acid containing a sequence of at least about 50, 100, 200, or 300 nucleotides that is identical to a contiguous sequence in SEQ ID NO: 9-11, 21-24, 37-39, or 49-51, wherein the presence of a sequence that is identical to a contiguous sequence in SEQ ID NO: 9-11, 21-24, 37-39, or 49-51 is indicative of the presence of NLS0109 (SEQ ID NO: 9-11), NL0020 (SEQ ID NO: 21-24) NLS0017 (SEQ ID NO: 37-39) or NLS0089 (SEQ ID NO: 49-51) in the sample. Such nucleic acid analyses include, but are not limited to, techniques based on nucleic acid hybridization, polymerase chain reactions, mass spectroscopy, nanopore based detection, branched DNA analyses, combinations thereof, and the like. One example of such a nucleic acid analysis is a qPCR Locked Nucleic Acid (LNA) based assay described herein. Such analysis can be used to detect Methylobacterium strains present at a concentration (CFU/gm of sample) of 10³, 10⁴, 10⁵, 10⁶ or more.

Also provided herein are methods of identifying and/or isolating Methylobacterium sp. that can inhibit growth of a plant pathogenic fungus by assaying for the presence of nucleic acid sequences contained in SEQ ID NO: SEQ ID NO: 9-11, 21-24, 37-39, or 49-51 in the Methylobacterium sp. In certain embodiments, such methods can comprise subjecting a candidate Methylobacterium sp. to a nucleic acid analysis technique and determining that the sample contains one or more nucleic acid containing a sequence of at least about 50, 100, 200, or 300 nucleotides that is identical to a contiguous sequence in SEQ ID NO: 9-11, 21-24, 37-39, or 49-51, wherein the presence of a sequence that is identical to a contiguous sequence in SEQ ID NO: 9-11, 21-24, 37-39, or 49-51 indicates that the candidate Methylobacterium sp. that can inhibit growth of a plant pathogenic fungus. Such nucleic acid analyses include, but are not limited to, techniques based on nucleic acid hybridization, polymerase chain reactions, mass spectroscopy, nanopore based detection, branched DNA analyses, combinations thereof, and the like.

In certain embodiments, a Methylobacterium that is NLS0109, a derivative thereof, or a Methylobacterium related to NLS0109, is used in any of the aforementioned compositions, uses thereof, or methods to inhibit growth of a plant pathogenic fungus, where the use and/or method comprises control of any of the pathogens or diseases, in any of the crops, through any of the modes of application set forth in Table 3. In certain embodiments of any of the aforementioned compositions, uses thereof, or methods, the Methylobacterium is NLS0109, a derivative thereof, or a Methylobacterium related to NLS0109, the plant pathogenic fungus that is inhibited is Fusarium graminearum, Septoria tritici, Stagonospora nodorum, Rhizoctonia solani, a Fusarium spp., Magnaportha grisea, Pyrenophora tritici-repentis, Microdochium nivale, Blumeria graminis f. sp. tritici, or a Pythium sp., and the plant or plant part is a wheat plant or plant part. In certain embodiments of any of the aforementioned compositions, uses thereof, or methods, the Methylobacterium is NLS0109, a derivative thereof, or a Methylobacterium related to NLS0109, the plant pathogenic fungus that is inhibited is Cercospora zeae-maydis, Colletotrichum graminicola, Fusarium graminearum, Fusarium verticillioides, Gibberella zeae, Rhizoctonia solani, Stenocarpella maydis, a Pythium sp., Sclerospora graminicola or Sclerophthora macrospora, and the plant or plant part is a corn plant or corn plant part. In certain embodiments of any of the aforementioned compositions, uses thereof, or methods, the Methylobacterium is NLS0109, a derivative thereof, or a Methylobacterium related to NLS0109, the plant pathogenic fungus that is inhibited is Sclerotinia sclerotiorum, Cercospora sojina, Cercospora kikuchii, Fusarium spp., Rhizoctonia solani, Septoria glycines, Fusarium virguliforme, Pythium spp., Peronospora manshurica, or Phytophthora sojae, and the plant or plant part is a soybean plant or soybean plant part.

In certain embodiments of any of the aforementioned compositions, uses thereof, or methods, the Methylobacterium is NLS0109, a derivative thereof, or a Methylobacterium related thereto and a second Methylobacterium selected from NLS0020, NLS0017, NLS0089, a derivative thereof, or a Methylobacterium related thereto, is included in the composition. In certain embodiments, the combination of NLS0109 with a second Methylobacterium selected from NLS0020, NLS0017, NLS0089, a derivative thereof, or a Methylobacterium related thereto that is used in any of the aforementioned compositions, uses thereof, or methods comprises any of the combinations of Methylobacterium, crops, pathogens, diseases, and/or modes of application set forth in Table 3. For example, in certain embodiments set forth in Table 3, the Methylobacterium is NLS0109, a derivative thereof, or a Methylobacterium related thereto and a second Methylobacterium is NLS0020,, a derivative thereof, or a Methylobacterium related thereto, the crop is wheat, the pathogen is Fusarium graminearum, Septoria tritici; Stagonospora nodorum, a Pythium spp., Rhizoctonia solani, a Fusarium spp., Blumeria graminis f. sp. tritici, Magnaporthe grisea, Pyrenophora tritici-repentis, or Microdochium nival, and application is to a seed in-furrow; foliar; or any combination thereof, where isolates are used alone or in combination for any treatment, including NLS0020 applied in-furrow and NLS0109 applied foliar. In certain embodiments, the Methylobacterium is NLS0109, a derivative thereof, or a Methylobacterium related thereto and a second Methylobacterium selected from NLS0020, NLS0017, NLS0089, a derivative thereof, or a Methylobacterium related thereto, the plant pathogenic fungus that is inhibited is Fusarium graminearum, Septoria tritici, Stagonospora nodorum, Rhizoctonia solani, a Fusarium spp., Magnaportha grisea, Pyrenophora tritici-repentis, Microdochium nivale, Blumeria graminis f. sp. tritici, or a Pythium sp., and the plant or plant part is a wheat plant or plant part. In certain embodiments, the Methylobacterium is NLS0109, a derivative thereof, or a Methylobacterium related thereto and a second Methylobacterium selected from NLS0020, NLS0021, NLS0017, NLS0089, a derivative thereof, or a Methylobacterium related thereto, the plant pathogenic fungus that is inhibited is Cercospora zeae-maydis, Colletotrichum graminicola, Fusarium graminearum, Fusarium verticillioides, Gibberella zeae, Rhizoctonia solani, Stenocarpella maydis, a Pythium sp., Sclerospora graminicola or Sclerophthora macrospora, and the plant or plant part is a corn plant or corn plant part. In certain embodiments, the Methylobacterium is NLS0109, a derivative thereof, or a Methylobacterium related thereto and a second Methylobacterium selected from NLS0020, NLS0064, NLS0017, NLS0089, a derivative thereof, or a Methylobacterium related thereto, the plant pathogenic fungus that is inhibited is Sclerotinia sclerotiorum, Cercospora sojina, Cercospora kikuchii, Fusarium spp., Rhizoctonia solani, Septoria glycines, Fusarium virguliforme, Pythium spp., Peronospora manshurica, or Phytophthora sojae, and the plant or plant part is a soybean plant or soybean plant part.

In certain embodiments of any of the aforementioned compositions, uses thereof, or methods, the plant or plant part comprises a fungal inhibitory amount of the Methylobacterium. In certain embodiments, a fungal inhibitory amount of the Methylobacterium applied to a plant part (e.g., a seed) is about 1.0×10³, 1.0×10⁴, or 1.0×105 to about 1.0×10⁷ or 1.0×10⁸ CFUs of PPFM bacteria/plant part (e.g., a seed). In certain embodiments, the Methylobacterium is heterologous to the plant or plant part. In certain embodiments of any of the aforementioned plant parts, the plant part is a leaf, a stem, a flower, a root, a tuber, or a seed.

Also provided are methods of making any of the aforementioned compositions containing the Methylobacterium that inhibit growth of a plant pathogenic fungus that comprise combining a Methylobacterium that inhibit growth of a plant pathogenic fungus with an agriculturally acceptable excipient and/or with an agriculturally acceptable adjuvant. In certain embodiments, the Methylobacterium has: (i) at least one gene that encodes a 16S RNA having at least 97%, 98%, 99%, 99.5%, or 100% sequence identity to SEQ ID NO: 8; (ii) has in its genome one or more polynucleotide marker fragments of at least 50, 60, 100, 120, 180, 200, 240, or 300 nucleotides of SEQ ID NOS: 9-11; or (iii) has in its genome one or more marker fragments comprising a sequence having at least 98%, 99%, or 99.5% sequence identity across the entire length of SEQ ID NOS: 9-11; or the Methylobacterium is NLS0109 or a derivative thereof. In certain embodiments of the methods, the Methylobacterium sp. is selected from the group consisting of M. aminovorans, M. extorquens, M. fujisawaense, M. mesophilicum, M. radiotolerans, M. rhodesianum, M. nodulans, M. phyllosphaerae, M thiocyanatum, and M. oryzae. In certain embodiments of the methods, the Methylobacterium is not M. radiotolerans or M. oryzae. In certain embodiments of the methods, the Methylobacterium is NLS0109, a derivative thereof, or a Methylobacterium related to NLS0109. In certain embodiments of any of the aforementioned methods, the composition further comprises Methylobacterium strain NLS0017, NLS0020, NLS0021, NLS0042, NLS0064, NLS0066, NLS0089, a derivative thereof, or a Methylobacterium sp. related thereto. In certain embodiments of any of the aforementioned methods, the Methylobacterium is NLS0109, a derivative thereof, or a Methylobacterium related to NLS0109. In certain embodiments, the plant or plant part is a soybean plant or soybean plant part. In certain embodiments, the plant or plant part is selected from the group consisting of a rice, wheat, corn, barley, millet, sorghum, oat, and rye plant or plant part. In certain embodiments of the methods, the plant pathogenic fungus is selected from the group consisting of a Blumeria sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. and a Verticillium sp.

In certain embodiments of any of the aforementioned compositions, uses thereof, or methods, the mono- or co-culture of Methylobacterium is adhered to a solid substance. In certain embodiments of the methods, the Methylobacterium that is adhered to the solid substance is combined with a liquid to form a composition that is a colloid. In certain embodiments of the methods, the colloid is a gel. In certain embodiments of the methods, the mono- or co-culture of Methylobacterium adhered to the solid substance is provided by culturing the Methylobacterium in the presence of the solid substance. In certain embodiments of the methods, the composition comprises an emulsion. In certain embodiments of the methods, the Methylobacterium is provided by culturing the Methylobacterium in an emulsion. In any of the aforementioned embodiments, the plant pathogenic fungus that is inhibited can be in its anamorphic form, its teleomorphic form, or in both its anamorphic and telcomorphic forms. In any of the aforementioned embodiments, the composition can further comprise an antifungal compound selected from the group consisting of an azole, dithiocarbamate, strobilurin, and benzimidazole. In certain embodiments, the azole is ipconazole.

Also provided are methods for controlling a plant pathogenic fungus that comprise applying any of the aforementioned compositions as a first composition that contains one or more Methylobacterium that inhibit growth of a plant pathogenic fungus to a plant or a plant part, to soil where a plant is grown, to soil where a plant part such as a seed is deposited, or any combination thereof, in an amount that provides for inhibition of infection by the plant pathogenic fungus in the plant, plant part, or a plant obtained therefrom relative to infection of a control plant, plant part, or plant obtained therefrom that had not received an application of the composition or been grown in soil treated with the composition. In certain embodiments, the Methylobacterium: (i) has at least one gene that encodes a 16S RNA having at least 97%, 98%, 99%, 99.5%, or 100% sequence identity to SEQ ID NO: 8;; (ii) has in its genome one or more polynucleotide marker fragments of at least 50, 60, 100, 120, 180, 200, 240, or 300 nucleotides of SEQ ID NOS: 9-11; or (iii) has in its genome one or more marker fragments comprising a sequence having at least 98%, 99%, or 99.5% sequence identity across the entire length of SEQ ID NOS: 9-11; or the Methylobacterium is NLS0109, a derivative thereof, or is a Methylobacterium related to NLS0109. In certain embodiments, the Methylobacterium contains in its genome one or more marker fragments having a sequence of SEQ ID NOS: 9-11.

In certain embodiments of any of the aforementioned methods or uses, the application of the composition provides for at least 40%, 50%, 75%, at least 85%, or at least 95% inhibition of a plant pathogenic fungal infection in the plant, plant part, or a plant derived therefrom relative to infection of the control plant, plant part, or plant obtained therefrom. In certain embodiments of the methods, the plant part is selected from the group consisting of a leaf, a stem, a flower, a root, a tuber, and a seed. In certain embodiments of the methods, the method further comprises the step of harvesting at least one plant part selected from the group consisting of a leaf, a stem, a flower, a root, a tuber, or a seed from the plant or plant part. In certain embodiments of the methods, the mycotoxin levels in the plant part are reduced by at least 50%, at least 75%, at least 85%, or at least 95% relative to a plant part obtained from the control plant, plant part, or plant obtained therefrom. In certain embodiments of the aforementioned methods, the method further comprises obtaining a processed food or feed composition from the plant or plant part. In certain embodiments of the aforementioned methods, the mycotoxin levels in the processed food or feed composition are reduced by at least 50%, at least 75%, at least 85%, or at least 95% relative to a processed food or feed composition obtained from the control plant, plant part, or plant obtained therefrom. In certain embodiments, a fungal inhibitory amount of the Methylobacterium is applied to the plant part. In certain embodiments, the fungal inhibitory amount of the Methylobacterium applied to a plant part (e.g., a seed) is about 1.0×10³, 1.0×10⁴, or 1.0×10⁵ to about 1.0×10⁷, 1.0×10⁸, 1.0×10⁹, or 1.0×10¹⁰ CFUs of Methylobacterium/plant part (e.g., a seed). In certain embodiments, the Methylobacterium is heterologous to the plant or plant part. In certain embodiments of any of the aforementioned methods, the plant part is a leaf, a stem, a flower, a root, a tuber, or a seed. In certain embodiments of the methods, the Methylobacterium is NLS0109, a derivative thereof, or a Methylobacterium related to NLS0109. In certain embodiments of any of the aforementioned methods, the composition further comprises Methylobacterium strain NLS0017, NLS0020, NLS0021, NLS0042, NLS0064, NLS0066, NLS0089, a derivative thereof, or a Methylobacterium sp. related thereto. In certain embodiments, a derivative of NLS0020, NLS0017, or NLS0089, or a Methylobacterium sp. related to NLS0020, NLS0017, or NLS0089, can be identified by the presence of one or more marker fragments having a sequence of SEQ ID NO:21-24, 37-39, or 49-51. In certain embodiments of any of the aforementioned methods, the Methylobacterium is NLS0109 or a derivative thereof. In certain embodiments, the plant or plant part is a soybean plant or soybean plant part, or a peanut plant or peanut plant part. In certain embodiments, the plant or plant part is selected from the group consisting of a rice, wheat, corn, barley, millet, sorghum, oat, and rye plant or plant part.

In certain embodiments, the methods can further comprise applying a second composition to soil where the plant is grown, wherein the second composition comprises Methylobacterium strain NLS0017, NLS0020, NLS0021, NLS0042, NLS0064, NLS0066, NLS0089, a derivative thereof, or a Methylobacterium sp. related thereto. In certain embodiments, the second composition is applied to the soil in furrow with the plant part or with a seed from which the plant is grown. In certain embodiments, the first composition is applied to foliage of the plant after application of the second composition to the soil. In certain embodiments, the first composition comprises NLS0109, a derivative thereof, or a Methylobacterium sp. related thereto; and the second composition comprises Methylobacterium strain NLS0020, NLS0021, NLS0017, a derivative thereof, or a Methylobacterium sp. related thereto. In certain embodiments, the first composition comprises NLS0109, a derivative thereof, or a Methylobacterium sp. related thereto and a strobilurin fungicide; and the second composition comprises Methylobacterium strain NLS0020, NLS0021, NLS0017, a derivative thereof, or a Methylobacterium sp. related thereto.

In certain embodiments, the methods can further comprise applying a fungicide to a plant, plant part or soil. In such embodiments, the fungicide can be applied simultaneously with one or more of the Methylobacterium sp. or may be applied as a separate composition to a plant or plant part. In certain embodiments, the fungicide is a strobilurin, a phenylamide, a thiazole-carboxamide, or a piperidinyl thiazole isoxazoline fungicide. In certain embodiments, the strobilurin fungicide is selected from the group consisting of azoxystrobin, pyraclostrobin, fluoxystrobin, and trifloxystrobin. In certain embodiments, the strobilurin fungicide is pyraclostrobin. In certain embodiments, the fungicide is metalaxyl, mefenoxam, ethaboxam or oxathiapiprolin. In certain embodiments of any of the aforementioned methods, the combination of Methylobacterium, crop, pathogen, disease, and/or antifungal compounds is as set forth in Table 4. In certain embodiments, the compositions provided herein or the plants or plant parts treated with the compositions can further comprise one or more of the aforementioned fungicides.

In certain embodiments, the Methylobacterium is NLS0109, a derivative thereof, or a Methylobacterium related to NLS0109 and the plant pathogenic fungus is a Fusarium sp., a Rhizoctonia sp., a Colletotrichum sp., a Cercospora sp., a Septoria sp., a Pythium sp., a Puccinia sp. or a Sclerotinia sp. In certain embodiments, the Methylobacterium is NLS0109, a derivative thereof, or a Methylobacterium related to NLS0109, the plant pathogenic fungus that is inhibited is Fusarium graminearum, Septoria tritici, Stagonospora nodorum, Rhizoctonia solani, a Fusarium spp., Magnaportha grisea, Pyrenophora tritici-repentis, Microdochium nivale, Blumeria graminis f. sp. tritici, or a Pythium sp., and the plant or plant part is a wheat plant or plant part. In certain embodiments, the Methylobacterium is NLS0109, a derivative thereof, or a Methylobacterium related to NLS0109, the plant pathogenic fungus that is inhibited is Cercospora zeae-maydis, Colletotrichum graminicola, Fusarium graminearum, Fusarium verticillioides, Gibberella zeae, Rhizoctonia solani, Stenocarpella maydis, a Pythium sp., Sclerospora graminicola or Sclerophthora macrospora, and the plant or plant part is a corn plant or corn plant part. In certain embodiments, the Methylobacterium is NLS0109, a derivative thereof, or a Methylobacterium related to NLS0109, the plant pathogenic fungus that is inhibited is Sclerotinia sclerotiorum, Cercospora sojina, Cercospora kikuchii, Fusarium spp., Rhizoctonia solani, Septoria glycines, Fusarium virguliforme, Pythium spp., Peronospora manshurica, or Phytophthora sojae, and the plant or plant part is a soybean plant or soybean plant part.

Also provided are isolated Methylobacterium that inhibit growth of a plant pathogenic fungus. In certain embodiments, the Methylobacterium sp. that inhibits growth of a plant pathogenic fungus has a 16S nucleic acid sequence having at least 97%, 98%, 99%, 99.5%, or 100% sequence identity to SEQ ID NO: 8; or the Methylobacterium is NLS0109 or a derivative thereof. In certain embodiments, the isolated Methylobacterium has in its genome one or more polynucleotide marker fragments of at least 50, 60, 100, 120, 180, 200, 240, or 300 nucleotides of SEQ ID NOS: 9-11. In certain embodiments, the isolated Methylobacterium has in its genome one or more marker fragments comprising a sequence having at least 98%, 99%, or 99.5% sequence identity across the entire length of SEQ ID NOS: 9-11. In certain embodiments, the plant pathogenic fungus is selected from the group consisting of a Blumeria sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Scleroplithora, a Sclerospora sp., a Sclerotium sp., a Sclerotinia sp., a Septoria sp., a Stagonospora sp., a Stenocarpella sp. and a Verticillium sp.

In any of the aforementioned embodiments, the plant pathogenic fungi that is inhibited can be in its anamorphic form, its telcomorphic form, or in both its anamorphic and telcomorphic forms. The use of any of the aforementioned isolated Methylobacterium to inhibit growth of any one of the aforementioned plant pathogenic fungi is also provided herein

### BRIEF DESCRIPTION OF THE DRAWINGS

The accompanying drawings, which are incorporated in and form a part of the specification, illustrate certain embodiments of the present disclosure. In the drawings:

FIG. 1 is a bar chart showing suppression of soybean white mold wilt symptom severity by NLS0109. White bars represent the mean for mock-inoculated controls; gray bars represent the mean for all inoculated plants within each treatment group. Means are calculated from three independent experiments with eight replicated pots per experiment (n=24). Errors bars represent±one SEM. Severity was rated on a 0-5 scale, with 0=no symptom development and 5=dead plant.

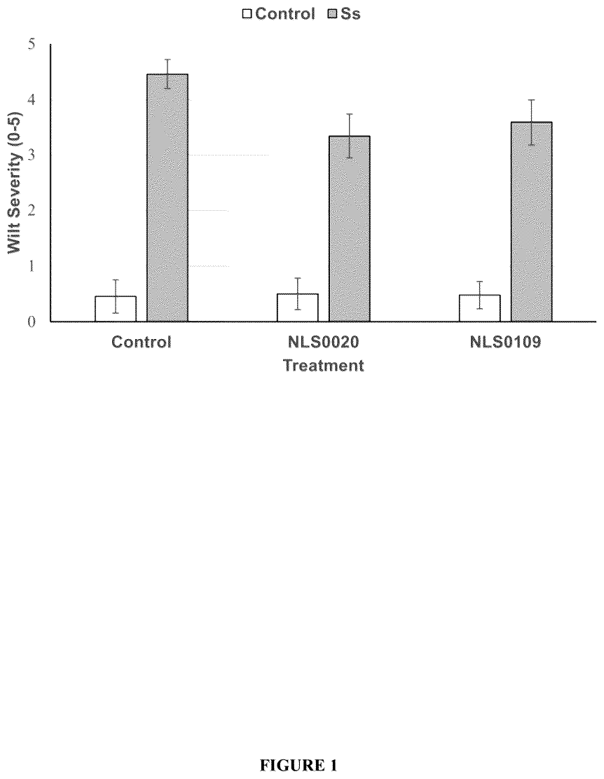

FIG. 2 is a bar chart showing suppression of soybean white mold lesion length development by NLS0109. White bars represent the mean for mock-inoculated controls; gray bars represent the mean for all inoculated plants within each treatment group. Means are calculated from three independent experiments with eight replicated pots per experiment (n=24). Errors bars represent±one SEM. Lesion length was measured to the nearest millimeter using a ruler held against the infected plant.

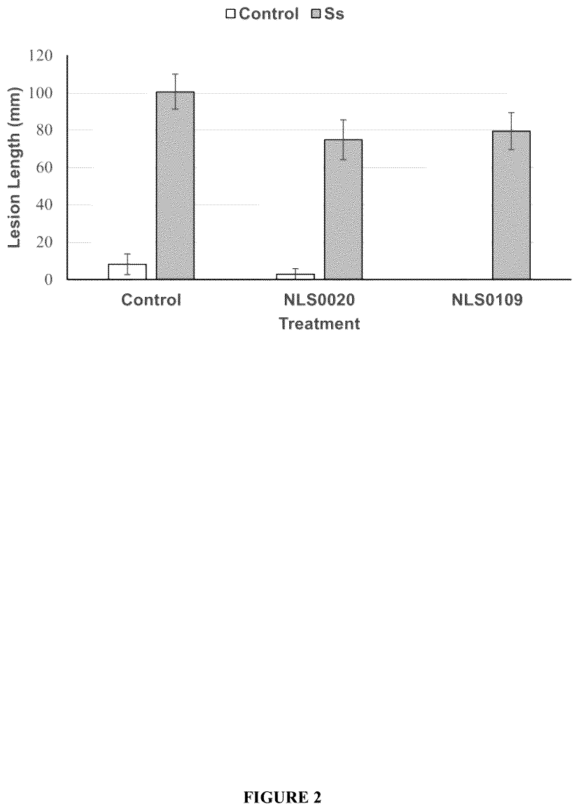

FIG. 3 is a bar chart showing the additive effect on reduction of late gray leaf spot disease severity in corn of NLS0020 in-furrow application followed by NL0109 foliar application. Applications were made during the 2015 field season at a trial site in Bethel, MO. NLS0020 was applied in-furrow at a rate of 1,250 mL/A and NLS0109 was applied at a foliar rate of 5,000 mL/A.

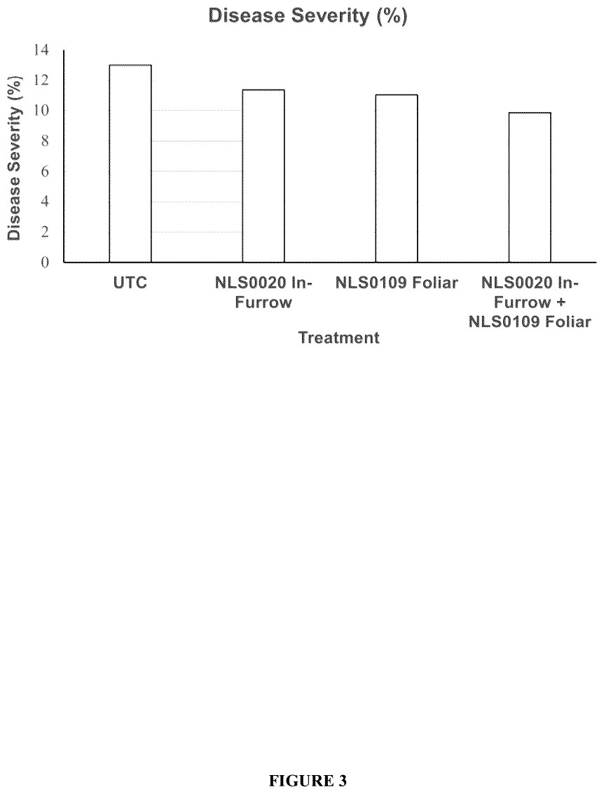

FIG. 4 is a line graph showing the progress of gray leaf spot disease severity over time in corn plants treated with PPFM strains, various commercial fungicides, and combinations thereof. Groups of ten plants were treated with various PPFM and fungicide treatments representing standard disease management practices or alternate PPFM treatment regimes alone or in combination with conventional chemistries. Disease incidence and severity data was collected at 14-, 21-, and 28-days post-inoculation and used to calculate disease index values [(incidenc *severity)/100].

### DESCRIPTION

### Definitions

As used herein, the phrases “adhered thereto” and “adherent” refer to Methylobacterium that are associated with a solid substance by growing, or having been grown, on a solid substance.

As used herein, the phrase “agriculturally acceptable adjuvant” refers to a substance that enhances the performance of an active agent in a composition comprising a mono-culture or co-culture of Methylobacterium for treatment of plants and/or plant parts.

As used herein, the phrase “agriculturally acceptable excipient” refers to an essentially inert substance that can be used as a diluent and/or carrier for an active agent in a composition for treatment of plants and/or plant parts. In certain compositions, an active agent can comprise a mono-culture or co-culture of Methylobacterium.

As used herein, the phrase “derivatives thereof”, when used in the context of a Methylobacterium strain, or isolate refers to any strain or isolate that is obtained from a given Methylobacterium strain. Derivatives of a Methylobacterium strain include, but are not limited to, variants of the strain obtained by selection, variants of the strain selected by mutagenesis and selection, variants of the strains resulting from unselected mutations resulting from growth in culture over one or more generations, and genetically transformed strains obtained from a Methylobacterium strain.

As used herein, the term “Methylobacterium” refers to bacteria that are facultative methylotrophs of the genus Methylobacterium. The term Methylobacterium, as used herein, thus does not encompass includes species in the genera Methylobacter, Methylomonas, Methylomicrobium, Methylococcus, Methylosinus, Methylocystis, Methylosphaera, Methylocaldum, and Methylocella, which are obligate methanotrophs.

As used herein, the phrase “co-culture of Methylobacterium” refers to a Methylobacterium culture comprising at least two strains of Methylobacterium or at least two species of Methylobacterium.

As used herein, the term “cultivar” refers to any plant known only in cultivation and includes asexually propagated plants, sexually propagated plants, inbred lines, and hybrids.

As used herein, the phrase “contaminating microorganism” refers to microorganisms in a culture, fermentation broth, fermentation broth product, or composition that were not identified prior to introduction into the culture, fermentation broth, fermentation broth product, or composition.

As used herein, the term “emulsion” refers to a colloidal mixture of two immiscible liquids wherein one liquid is the continuous phase and the other liquid is the dispersed phase. In certain embodiments, the continuous phase is an aqueous liquid and the dispersed phase is liquid that is not miscible, or partially miscible, in the aqueous liquid.

As used herein, the phrase “essentially free of contaminating microorganisms” refers to a culture, fermentation broth, fermentation product, or composition where at least about 95% of the microorganisms present by amount or type in the culture, fermentation broth, fermentation product, or composition are the desired Methylobacterium or other desired microorganisms of pre-determined identity.

As used herein, the phrase “a fungal inhibitory concentration of the mono- or co-culture of Methylobacterium” is a concentration that provides for at least a 40%, 50%, 75%, at least 85%, or at least 95% inhibition of a plant pathogenic fungal infection in a plant, plant part, or a plant derived therefrom relative to infection of the control plant or plant part.

As used herein, the term “heterologous”, when used in the context of Methylobacterium that at least partially coats a plant or plant part, refers to a Methylobacterium that is not naturally associated with a plant or plant part of the same species as the plant or plant part that is at least partially coated with the Methylobacterium. In certain embodiments, the heterologous Methylobacterium that is used to at least partially coat a plant or plant part of a first plant species is a Methylobacterium that was isolated, or can be isolated, from a second and distinct plant species.

As used herein, the phrase “inanimate solid substance” refers to a substance which is insoluble or partially soluble in water or aqueous solutions and which is either non-living or which is not a part of a still-living organism from which it was derived.

As used herein, the phrase “a Methylobacterium related to NLS0109” can refer to a Methylobacterium that: (i) has at least one gene that encodes a 16S RNA having at least 97%, 98%, 99%, 99.5%, or 100% sequence identity to SEQ ID NO: 8;; (ii) has in its genome one or more polynucleotide marker fragments of at least 50, 60, 100, 120, 180, 200, 240, or 300 nucleotides of SEQ ID NOS: 9-11; or (iii) has in its genome one or more marker fragments comprising a sequence having at least 98%, 99%, or 99.5% sequence identity across the entire length of SEQ ID NOS: 9-11.

As used herein, the phrase “mono-culture of Methylobacterium” refers to a Methylobacterium culture consisting of a single strain of Methylobacterium.

As used herein, a “pesticide” refers to an agent that is insecticidal, fungicidal, nematocidal, bacteriocidal, or any combination thereof.

As used herein, the phrase “bacteriostatic agent” refers to agents that inhibit growth of bacteria but do not kill the bacteria.

As used herein, the phrase “pesticide does not substantially inhibit growth of the Methylobacterium” refers to any pesticide that when provided in a composition comprising a fermentation product comprising a solid substance wherein a mono-culture or co-culture of Methylobacterium is adhered thereto, results in no more than a 50% inhibition of

Methylobacterium growth when the composition is applied to a plant or plant part in comparison to a composition lacking the pesticide. In certain embodiments, the pesticide results in no more than a 40%, 20%, 10%, 5%, or 1% inhibition of Methylobacterium growth when the composition is applied to a plant or plant part in comparison to a composition lacking the pesticide.

As used herein, the term “PPFM bacteria” refers without limitation to bacterial species in the genus Methylobacterium other than M. nodulans.

As used herein, the phrase “solid substance” refers to a substance which is insoluble or partially soluble in water or aqueous solutions.

As used herein, the phrase “solid phase that can be suspended therein” refers to a solid substance that can be distributed throughout a liquid by agitation.

As used herein, the term “non-regenerable” refers to either a plant part or processed plant product that cannot be regenerated into a whole plant.

As used herein, the phrase “substantially all of the solid phase is suspended in the liquid phase” refers to media wherein at least 95%, 98%, or 99% of solid substance(s) comprising the solid phase are distributed throughout the liquid by agitation.

As used herein, the phrase “substantially all of the solid phase is not suspended in the liquid phase” refers to media where less than 5%, 2%, or 1% of the solid is in a particulate form that is distributed throughout the media by agitation.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Methylobacterium that Inhibit Plant Pathogenic Fungi, Compositions Comprising Methylobacterium that Inhibit Plant Pathogenic Fungi, Methods of Their Use, and Methods of Making

Various Methylobacterium that inhibit plant pathogenic fungi, compositions comprising these Methylobacterium, methods of using the compositions to inhibit plant pathogenic fungi, and methods of making the compositions are provided herein. As used herein, inhibition of the growth of a plant pathogenic fungus includes any measurable decrease in fungal growth, where fungal growth includes but is not limited to any measurable decrease in the numbers and/or extent of fungal cells, spores, conidia, or mycelia. As used herein, inhibition of infection by a plant pathogenic fungus and/or inhibition of the growth of a plant pathogenic fungus are also understood to include any measurable decrease in the adverse effects caused by fungal growth in a plant. Adverse effects of fungal growth in a plant include, but are not limited to, any type of plant tissue damage or necrosis, any type of plant yield reduction, any reduction in the value of the crop plant product, and/or production of undesirable fungal metabolites or fungal growth by-products including, but not limited to, mycotoxins. Plant pathogenic fungi that are inhibited by the compositions and Methylobacterium provided herein can be in their anamorphic form, their telcomorphic form, or in both their anamorphic and telcomorphic forms.

Methylobacterium and compositions comprising the same that inhibit growth of a plant pathogenic fungus are provided herein. In certain embodiments, the Methylobacterium or composition provides for at least about 25%, at least about 40%, at least about 50%, or at least about 75% inhibition of plant pathogenic fungal growth in comparison to a control treatment upon exposure to a plant pathogenic fungus. In certain embodiments, the plant pathogenic fungus that is inhibited is selected from the group consisting of a Blumeria sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora sp., a Sclerospora sp., a Sclerotium sp., a Sclerotinia sp., a Septoria sp., a Stagonospora sp., a Stenocarpella sp. and a Verticillium sp.

In certain embodiments, the plant pathogenic fungus that is inhibited is a Fusarium sp. In certain embodiments, the Fusarium sp. that is inhibited is selected from the group consisting of Fusarium graminearum, Fusarium verticillioides, Fusarium oxysporum, Fusarium virguliforme, and Fusarium solani. In certain embodiments, the isolated Methylobacterium is NLS0109, a derivative thereof, or a Methylobacterium related to NLS0109. In certain embodiments, Methylobacterium related to NLS0109 include, but are not limited to, (i) Methylobacterium sp. that inhibit growth of a plant pathogenic fungus and that have a gene encoding a 16S RNA sequence that has at least 97%, 98%, 99%, 99.5%, or 100% sequence identity to SEQ ID NO:8; (ii) a Methylobacterium has in its genome one or more polynucleotide marker fragments of at least 50, 60, 100, 120, 180, 200, 240, or 300 nucleotides of SEQ ID NOS: 9-11; or (iii) is a Methylobacterium that has in its genome one or more marker fragments comprising a sequence having at least 98%, 99%, or 99.5% sequence identity across the entire length of SEQ ID NOS: 9-11. In certain embodiments, the composition further comprises Methylobacterium strain NLS0017, NLS0020, NLS0021, NLS0042, NLS0064, NLS0066, NLS0089, a derivative thereof, or a Methylobacterium sp. related thereto. Plant pathogenic fungi that are inhibited by the compositions and Methylobacterium provided herein can be in their anamorphic form, their teleomorphic form, or in both their anamorphic and telcomorphic forms.

Also provided are compositions that comprise Methylobacterium that inhibit growth of a plant pathogenic fungus. In certain embodiments, the compositions further comprise an agriculturally acceptable excipient and/or an agriculturally acceptable adjuvant. In certain embodiments, the composition provides for at least about 25%, about 50%, or about 75% inhibition of plant pathogenic fungal growth in comparison to a control treatment upon exposure to a plant pathogenic fungus. In certain embodiments, the plant pathogenic fungus that is inhibited is selected from the group consisting of a Blumerid sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. and a Verticillium sp.

In certain embodiments, the plant pathogenic fungus that is inhibited is a Fusarium sp. In certain embodiments, the Fusarium sp., which is inhibited is selected from the group consisting of Fusarium graminearum, Fusarium verticillioides, Fusarium oxysporum, Fusarium virguliforme, and Fusarium solani. In certain embodiments of any of the aforementioned compositions, the composition comprises a solid substance wherein a mono-culture or co-culture of Methylobacterium is adhered thereto. In certain embodiments where the Methylobacterium is adhered to a solid substance, the composition comprises a colloid formed by the solid substance wherein a mono-culture or co-culture of Methylobacterium is adhered thereto and a liquid. In certain embodiments, the colloid is a gel. In certain embodiments of certain aforementioned compositions, composition is an emulsion that does not contain a solid substance. In certain embodiments of any of the aforementioned compositions, the Methylobacterium sp. that inhibits growth of a plant pathogenic fungus has a gene encoding a 16S RNA sequence that has at least 97%, 98%, 99%, 99.5%, or 100% sequence identity to SEQ ID NO:8. In certain embodiments of any of the aforementioned compositions, the Methylobacterium is NLS0109, a derivative thereof, or a Methylobacterium related to NLS0109. In certain embodiments of any of the aforementioned compositions, the composition further comprises Methylobacterium strain NLS0017, NLS0020, NLS0021, NLS0042, NLS0064, NLS0066, NLS0089, a derivative thereof, or a Methylobacterium sp. related thereto. In any of the aforementioned embodiments, the plant pathogenic fungi that are inhibited can be in their anamorphic form, their telcomorphic form, or in both their anamorphic and telcomorphic forms.

In certain embodiments, the Methylobacterium sp. that inhibit plant pathogenic fungi can be identified by testing newly isolated candidate Methylobacterium sp. for the presence of polymorphic nucleic acid, orthologous gene, or gene sequences that are present in Methylobacterium sp. provided herein that inhibit certain plant pathogenic fungi.

Various Methylobacterium sp. isolates provided herein are disclosed in Table 1.

TABLE 1
Methylobacterium sp. isolates

| | | USDA ARS |
|---|---|---|
| NLS | Origin | NRRL No.¹ |
| NLS0017 | Obtained from a peppermint plant grown | NRRL B-50931 |
| | in Saint Louis County, Missouri, USA | |
| NLS0020 | Obtained from a horse nettle plant grown | NRRL B-50930 |
| | in Saint Louis County, Missouri, USA | |
| NLS0021 | Obtained from a lettuce plant grown in | NRRL B-50939 |
| | Saint Louis Country, Missouri, USA | |
| NLS0042 | Obtained from a soybean plant grown in | NRRL B-50932 |
| | Saint Louis Country, Missouri, USA | |
| NLS0064 | Obtained from a corn plant grown in | NRRL B-50938 |
| | Saint Louis Country, Missouri, USA | |
| NLS0066 | Obtained from the corn hybrid | NRRL B-50940 |
| | “MC534” (Masters Choice 3010 | |
| | State Route 146 East Anna, IL 62906) | |
| NLS0089 | Obtained from a broccoli plant grown | NRRL B-50933 |
| | in Saint Louis County, Missouri, USA | |
| NLS0109 | Obtained from a Yucca filamentosa plant | NRRL B-67340 |
| | in Saint Louis Country, Missouri, USA | |

¹Deposit number for strain deposited with the AGRICULTURAL RESEARCH SERVICE CULTURE COLLECTION (NRRL) of the National Center for Agricultural Utilization Research, Agricultural Research Service, U.S. Department of Agriculture, 1815 North University Street, Peoria, Illinois 61604 U.S.A. under the terms of the Budapest Treaty on the International Recognition of the Deposit of Microorganisms for the Purposes of Patent Procedure. Methylocacterium sp. NRRL B-50930, NRRL B-50931, NRRL B-50932, NRRL B-50933, NRRL B-50938, NRRL B-50939, and NRRL B-50940 were deposited with NRRL on Mar. 12, 2014. Methylobacterium sp. NRL B-67340 was deposited with NRRL on Nov. 18, 2016. Subject to 37 CFR §1.808(b), all restrictions imposed by the depositor on the availability to the public of the deposited material will be irrevocably removed upon the granting of any patent from this patent application.

Also provided herein are methods for controlling a plant pathogenic fungus that comprise applying any of the aforementioned compositions comprising the Methylobacterium that are provided herein to a plant or a plant part in an amount that provides for inhibition of infection by the plant pathogenic fungus in the plant, plant part, or a plant obtained therefrom relative to infection of a control plant, plant part, or plant obtained therefrom that had not received an application of the composition. In certain embodiments, application of the composition provides for at least about 40%, at least about 50%, at least about 75%, at least about 85%, or at least about 95% inhibition of a plant pathogenic fungal infection in the plant, plant part, or a plant derived therefrom relative to infection of the control plant, plant part, or plant obtained therefrom. In certain embodiments, the plant part is selected from the group consisting of a leaf, a stem, a flower, a root, a tuber, and a seed. In certain embodiments, the method further comprises the step of harvesting at least one plant part selected from the group consisting of a leaf, a stem, a flower, a root, a tuber, or a seed from the plant or plant part. In certain embodiments of any of the aforementioned methods, the mycotoxin levels in the plant part are reduced by at least 50%, at least 75%, at least 85%, or at least 95% relative to a plant part obtained from the control plant, plant part, or plant obtained therefrom. In certain embodiments of any of the aforementioned methods, the methods further comprise obtaining a processed food or feed composition from the plant or plant part. In certain embodiments of the aforementioned methods, mycotoxin levels in the processed food or feed composition are reduced by at least 50%, at least 75%, at least 85%, or at least 95% relative to a processed food or feed composition obtained from the control plant, plant part, or plant obtained therefrom. In certain embodiments of any of the aforementioned methods, the composition comprises the Methylobacterium isolate NLS0109, a derivative thereof, or a Methylobacterium related to NLS0109. In certain embodiments of any of the aforementioned methods, the composition further comprises Methylobacterium strain NLS0017, NLS0020, NLS0021, NLS0042, NLS0064, NLS0066, NLS0089, a derivative thereof, or a Methylobacterium sp. related thereto. In certain embodiments of any of the aforementioned methods, the composition comprises a Methylobacterium sp. related to NLS0109 that: (i) has at least one gene encoding a 16S RNA that has at least 97%, 98%, 99%, 99.5%, or 100% sequence identity to SEQ ID NO: 8; (ii) has in its genome one or more polynucleotide marker fragments of at least 50, 60, 100, 120, 180, 200, 240, or 300 nucleotides of SEQ ID NOS: 9-11; or (iii) has in its genome one or more marker fragments comprising a sequence having at least 98%, 99%, or 99.5% sequence identity across the entire length of SEQ ID NOS: 9-11. In certain embodiments of any of the aforementioned methods, the composition comprises a Methylobacterium sp. related to NLS0017, NLS0020, NLS0021, NLS0042, NLS0064, NLS0066, or NLS0089 that has at least one gene encoding a 16S RNA that has at least 97%, 98%, 99%, 99.5%, or 100% sequence identity to SEQ ID NO:1, 2, 3, 4, 5, 6, or 7, respectively.

TABLE 2
16S RNA Sequences of Methylobacterium strains

| | SEQ | |
|---|---|---|
| | ID | |
| NLS | NO | 16S RNA encoding DNA sequence |
| NLS0017 | 1 | ggtgatccagccgcaggttcccctacggctaccttgttacgactt |
| | | caccccagtcgctgaccctaccgtggtcgcctgcctccttgcggt |
| | | tggcgcagcgccgtcgggtaagaccaactcccatggtgtgacggg |
| | | cggtgtgtacaaggcccgggaacgtattcaccgtggcatgctgat |
| | | ccacgattactagcgattccgccttcatgcactcgagttgcagag |
| | | tgcaatccgaactgagacggcttttggggatttgctccagatcgc |
| | | tccttcgcctcccactgtcaccgccattgtagcacgtgtgtagcc |
| | | catcccgtaagggccatgaggacttgacgtcatccacaccttcct |
| | | cgcggcttatcaccggcagtctccctagagtgcccaactgaatga |
| | | tggcaactaaggacgtgggttgcgctcgttgcgggacttaaccca |
| | | acatctcacgacacgagctgacgacagccatgcagcacctgtgtg |
| | | cgcgccaccgaagtggaccccaaatctctctgggtaacacgccat |
| | | gtcaaaggatggtaaggttctgcgcgttgcttcgaattaaaccac |
| | | atgctccaccgcttgtgcgggcccccgtcaattcctttgagtttt |
| | | aatcttgcgaccgtactccccaggcggaatgctcaaagcgttagc |
| | | tgcgctactgcggtgcaagcaccccaacagctggcattcatcgtt |
| | | tacggcgtggactaccagggtatctaatcctgtttgctccccacg |
| | | ctttcgcgcctcagcgtcagtaatggtccagttggccgccttcgc |
| | | caccggtgttcttgcgaatatctacgaatttcacctctacactcg |
| | | cagttccaccaacctctaccatactcaagcgtcccagtatcgaag |
| | | gccattctgtggttgagccacaggctttcacccccgacttaaaac |
| | | gccgcctacgcgccctttacgcccagtgattccgagcaacgctag |
| | | cccccttcgtattaccgcggctgctggcacgaagttagccggggc |
| | | ttattcctccggtaccgtcattatcgtcccggataaaagagcttt |
| | | acaaccctaaggccttcatcactcacgcggcatggctggatcagg |
| | | cttgcgcccattgtccaatattccccactgctgcctcccgtagga |
| | | gtctgggccgtgtctcagtcccagtgtggctgatcatcctctcag |
| | | accagctactgatcgtcgccttggtaggccgttaccccaccaact |
| | | agctaatcagacgcgggccgatcttccggcagtaaacctttcccc |
| | | aaaagggcgtatccggtattagccctagtttcccagggttattcc |
| | | gaaccagaaggcacgttcccacgcgttactcacccgtccgccgct |
| | | gaccccgaaaggcccgctcgacttgcatgtgttaagcctgccgcc |
| | | agcgttcgctctgagccaggatcaaactctc |
| NLS0020 | 2 | ggtgatccagccgcaggttcccctacggctaccttgttacgactt |
| | | caccccagtcgctgaccctaccgtggtcgcctgcctccttgcggt |
| | | tggcgcagcgccgtcgggtaagaccaactcccatggtgtgacggg |
| | | cggtgtgtacaaggcccgggaacgtattcaccgtggcatgctgat |
| | | ccacgattactagcgattccgccttcatgcactcgagttgcagag |
| | | tgcaatccgaactgagacggcttttggggatttgctccagatcgc |
| | | tccttcgcgtcccactgtcaccgccattgtagcacgtgtgtagcc |
| | | catcccgtaagggccatgaggacttgacgtcatccacaccttcct |
| | | cgcggcttatcaccggcagtctccctagagtgcccaactgaatga |
| | | tggcaactaaggacgtgggttgcgctcgttgcgggacttaaccca |
| | | acatctcacgacacgagctgacgacagccatgcagcacctgtgtg |
| | | cgcgccaccgaagtggaccccaaatctctctgggtaacacgccat |
| | | gtcaaaggatggtaaggttctgcgcgttgcttcgaattaaaccac |
| | | atgctccaccgcttgtgcgggcccccgtcaattcctttgagtttt |
| | | aatcttgcgaccgtactccccaggcggaatgctcaaagcgttagc |
| | | tgcgctactgcggtgcaagcaccccaacagctggcattcatcgtt |
| | | tacggcgtggactaccagggtatctaatcctgtttgctccccacg |
| | | ctttcgcgcctcagcgtcagtaatggtccagttggccgccttcgc |
| | | caccggtgttcttgcgaatatctacgaatttcacctctacactcg |
| | | cagttccaccaacctctaccatactcaagcgtcccagtatcgaag |
| | | gccattctgtggttgagccacaggctttcacccccgacttaaaac |
| | | gccgcctacgcgccctttacgcccagtgattccgagcaacgctag |
| | | cccccttcgtattaccgcggctgctggcacgaagttagccggggc |
| | | ttattcctccggtaccgtcattatcgtcccggataaaagagcttt |
| | | acaaccctaaggccttcatcactcacgcggcatggctggatcagg |
| | | cttgcgcccattgtccaatattccccactgctgcctcccgtagga |
| | | gtctgggccgtgtctcagtcccagtgtggctgatcatcctctcag |
| | | accagctactgatcgtcgccttggtaggccgttaccccaccaact |
| | | agctaatcagacgcgggccgatcttccggcagtaaacctttcccc |
| | | aaaagggcgtatccggtattagccctagtttcccagggttattcc |
| | | gaaccagaaggcacgttcccacgcgttactcacccgtccgccgct |
| | | gaccccgaagggcccgctcgacttgcatgtgttaagcctgccgcc |
| | | agcgttcgctctgagccaggatcaaactctc |
| NLS0021 | 3 | gagtttgatcctggctcagagcgaacgctggcggcaggcttaaca |
| | | catgcaagtcgaacgggcttcttcggaagtcagtggcagacgggt |
| | | gagtaacacgtgggaacgtgcccttcggttcggaataactcaggg |
| | | aaacttgagctaataccggatacgcccttatggggaaaggtttac |
| | | tgccgaaggatcggcccgcgtctgattagcttgttggtggggtaa |
| | | cggcctaccaaggcgacgatcagtagctggtctgagaggatgatc |
| | | agccacactgggactgagacacggcccagactcctacgggaggca |
| | | gcagtggggaatattggacaatgggcgcaagcctgatccagccat |
| | | gccgcgtgagtgatgaaggccttagggttgtaaagctcttttgtc |
| | | cgggacgataatgacggtaccggaagaataagccccggctaactt |
| | | cgtgccagcagccgcggtaatacgaagggggctagcgttgctcgg |
| | | aatcactgggcgtaaagggcgcgtaggcggccgattaagtcgggg |
| | | gtgaaagcctgtggctcaaccacagaattgccttcgatactggtt |
| | | ggcttgagaccggaagaggacagcggaactgcgagtgtagaggtg |
| | | aaattcgtagatattcgcaagaacaccagtggcgaaggcggctgt |
| | | ctggtccggttctgacgctgaggcgcgaaagcgtggggagcaaac |
| | | aggattagataccctggtagtccacgccgtaaacgatgaatgcca |
| | | gccgttggtctgcttgcaggtcagtggcgccgctaacgcattaag |
| | | cattccgcctggggagtacggtcgcaagattaaaactcaaaggaa |
| | | ttgacgggggcccgcacaagcggtggagcatgtggtttaattcga |
| | | agcaacgcgcagaaccttaccatcccttgacatggcatgttacct |
| | | cgagagatcggggatcctcttcggaggcgtgcacacaggtgctgc |
| | | atggctgtcgtcagctcgtgtcgtgagatgttgggttaagtcccg |
| | | caacgagcgcaacccacgtccttagttgccatcattcagttgggc |
| | | actctagggagactgccggtgataagccgcgaggaaggtgtggat |
| | | gacgtcaagtcctcatggcccttacgggatgggctacacacgtgc |
| | | tacaatggcggtgacagtgggacgcgaaaccgcgaggttgagcaa |
| | | atccccaaaagccgtctcagttcggattgcactctgcaactcggg |
| | | tgcatgaaggcggaatcgctagtaatcgtggatcagcacgccacg |
| | | gtgaatacgttcccgggccttgtacacaccgcccgtcacaccatg |
| | | ggagttggtcttacccgacggcgctgcgccaaccgcaagggggca |
| | | ggcgaccacggtagggtcagcgactggggtgaagtcgtaacaagg |
| | | tagccgtaggggaacctgcggctggatcacct |
| NLS0042 | 4 | ggtgatccagccgcaggttcccctacggctaccttgttacgactt |
| | | caccccagtcgctgaccctaccgtggtcgcctgcctccttgcggt |
| | | tggcgcagcgccgtcgggtaagaccaactcccatggtgtgacggg |
| | | cggtgtgtacaaggcccgggaacgtattcaccgtggcgtgctgat |
| | | ccacgattactagcgattccgccttcatgcacccgagttgcagag |
| | | tgcaatccgaactgagacggtttttggggatttgctccacctcgc |
| | | ggcttcgcgtcccactgtcaccgccattgtagcacgtgtgtagcc |
| | | catcccgtaagggccatgaggacttgacgtcatccacaccttcct |
| | | cgcggcttatcaccggcagtctccctagagtgcccaactgaatga |
| | | tggcaactaaggacgtgggttgcgctcgttgcgggacttaaccca |
| | | acatctcacgacacgagctgacgacagccatgcagcacctgtgtg |
| | | cacgcctccgaagaggatccccgatctctcgaggtaacatgccat |
| | | gtcaagggatggtaaggttctgcgcgttgcttcgaattaaaccac |
| | | atgctccaccgcttgtgcgggcccccgtcaattcctttgagtttt |
| | | aatcttgcgaccgtactccccaggcggaatgcttaatgcgttagc |
| | | ggcgccactgacctgcaagcaggccaacggctggcattcatcgtt |
| | | tacggcgtggactaccagggtatctaatcctgtttgctccccacg |
| | | ctttcgcgcctcagcgtcagaaccggaccagacagccgccttcgc |
| | | cactggtgttcttgcgaatatctacgaatttcacctctacactcg |
| | | cagttccgctgtcctcttccggtctcaagccaaccagtatcgaag |
| | | gcaattctgtggttgagccacaggctttcacccccgacttaatcg |
| | | gccgcctacgcgccctttacgcccagtgattccgagcaacgctag |
| | | cccccttcgtattaccgcggctgctggcacgaagttagccggggc |
| | | ttattcttccggtaccgtcattatcgtcccggacaaaagagcttt |
| | | acaaccctaaggccttcatcactcacgcggcatggctggatcagg |
| | | cttgcgcccattgtccaatattccccactgctgcctcccgtagga |
| | | gtctgggccgtgtctcagtcccagtgtggctgatcatcctctcag |
| | | accagctactgatcgtcgccttggtaggccgttaccccaccaaca |
| | | agctaatcagacgcgggccgatccttcggcagtaaacctttcccc |
| | | aaaagggcgtatccggtattagctcaagtttccctgagttattcc |
| | | gaaccgaagggtacgttcccacgtgttactcacccgtctgccact |
| | | gacacccgaaggtgcccgttcgacttgcatgtgttaagcctgccg |
| | | ccagcgttcgctctgagccaggatcaaactctc |
| NLS0064 | 5 | ggtgatccagccgcaggttcccctacggctaccttgttacgacttcaccccagtcgctgaccctaccg |
| | | tggtcgcctgcctccagtcgagcaagctcgatttggttggcgcagcgccgtcgggtaagaccaactcc |
| | | catggtgtgacgggcggtgtgtacaaggcccgggaacgtattcaccgtggcatgctgatccacgatta |
| | | ctagcgattccgccttcatgcacgcgagttgcagcgtgcaatccgaactgagacggcttttggagatt |
| | | ggctccgggtcaccccttcgcgtcccactgtcaccgccattgtagcacgtgtgtagcccatcccgtaa |
| | | gggccatgaggacttgacgtcatccacaccttcctcgcggcttatcaccggcagtctccctagagtgc |
| | | ccaaccaaatgatggcaactaaggacgtgggttgcgctcgttgcgggacttaacccaacatctcacga |
| | | cacgagctgacgacagccatgcagcacctgtgtgcgcgcccccgaaggggacctggaatctctcccag |
| | | taacacgccatgtcaaaggatggtaaggttctgcgcgttgcttcgaattaaaccacatgctccaccgc |
| | | ttgtgcgggcccccgtcaattcctttgagttttaatcttgcgaccgtactccccaggcggaatgctta |
| | | atgcgttagctgcgctactgcggtgcatgcaccccaacagctagcattcatcgtttacggcgtggact |
| | | accagggtatctaatcctgtttgctccccacgctttcgcgcctcagcgtcagtaatggtccagttggc |
| | | cgccttcgccaccggtgttcttgcgaatatctacgaatttcacctctacactcgcagttccaccaacc |
| | | tctaccatactcaagcgtcccagtatcgaaggccattctgtggttgagccacaggctttcacccccga |
| | | cttaaaacgccgcctacgcgccctttacgcccagtgattccgagcaacgctagcccccttcgtattac |
| | | cgcggctgctggcacgaagttagccggggcttattcctccggtaccgtcattatcgtcccggagaaaa |
| | | gagctttacaaccctaaggccgtcatcactcacgcggcatggctggatcaggcttgcgcccattgtcc |
| | | aatattccccactgctgcctcccgtaggagtctgggccgtgtctcagtcccagtgtggctgatcatcc |
| | | tctcagaccagctactgatcgtcgccttggtaggccgttaccccaccaacaagctaatcagacgcggg |
| | | ccgatcctccggcagtaaacctttctgccaaagcacgtatccggtattagccctagtttcccagggtt |
| | | atcccagaccggagggcacgttcccacgtgttactcacccgtctgccactcaccttgcggtgcgttcg |
| | | acttgcatgtgttaagcctgccgccagcgttcgctctgagccaggatcaaactctc |
| NLS0066 | 6 | gagtttgatcctggctcagagcgaacgctggcggcaggcttaacacatgcaagtcgaacgcaccgcaa |
| | | ggtgagtggcagacgggtgagtaacacgtgggaacgtgccctccggtctgggataaccctgggaaact |
| | | agggctaataccggatacgtgctttggcagaaaggtttactgccggaggatcggcccgcgtctgatta |
| | | gcttgttggtggggtaacggcctaccaaggcgacgatcagtagctggtctgagaggatgatcagccac |
| | | actgggactgagacacggcccagactcctacgggaggcagcagtggggaatattggacaatgggcgca |
| | | agcctgatccagccatgccgcgtgagtgatgacggccttagggttgtaaagctcttttctccgggacg |
| | | ataatgacggtaccggaggaataagccccggctaacttcgtgccagcagccgcggtaatacgaagggg |
| | | gctagcgttgctcggaatcactgggcgtaaagggcgcgtaggcggcgttttaagtcgggggtgaaagc |
| | | ctgtggctcaaccacagaatggccttcgatactgggacgcttgagtatggtagaggttggtggaactg |
| | | cgagtgtagaggtgaaattcgtagatattcgcaagaacaccggtggcgaaggcggccaactggaccat |
| | | tactgacgctgaggcgcgaaagcgtggggagcaaacaggattagataccctggtagtccacgccgtaa |
| | | acgatgaatgctagctgttggggtgcatgcaccgcagtagcgcagctaacgcattaagcattccgcct |
| | | ggggagtacggtcgcaagattaaaactcaaaggaattgacgggggcccgcacaagcggtggagcatgt |
| | | ggtttaattcgaagcaacgcgcagaaccttaccatcctttgacatggcgtgttactgggagagattcc |
| | | aggtccccttcgggggcgcgcacacaggtgctgcatggctgtcgtcagctcgtgtcgtgagatgttgg |
| | | gttaagtcccgcaacgagcgcaacccacgtccttagttgccatcatttggttgggcactctagggaga |
| | | ctgccggtgataagccgcgaggaaggtgtggatgacgtcaagtcctcatggcccttacgggatgggct |
| | | acacacgtgctacaatggcggtgacagtgggacgcgaaggggtgacccggagccaatctccaaaagcc |
| | | gtctcagttcggattgcacgctgcaactcgcgtgcatgaaggcggaatcgctagtaatcgtggatcag |
| | | catgccacggtgaatacgttcccgggccttgtacacaccgcccgtcacaccatgggagttggtcttac |
| | | ccgacggcgctgcgccaaccaaatcgagcttgctcgactggaggcaggcgaccacggtagggtcagcg |
| | | actggggtgaagtcgtaacaaggtagccgtaggggaacctgcggctggatcacctc |
| NLS0089 | 7 | gagtttgatcctggctcagagcgaacgctggcggcaggcttaaca |
| | | catgcaagtcgaacgggcttcttcggaagtcagtggcagacgggt |
| | | gagtaacacgtgggaacgtgcccttcggttcggaataactcaggg |
| | | aaacttgagctaataccggatacgcccttacggggaaaggtttac |
| | | tgccgaaggatcggcccgcgtctgattagcttgttggtggggtaa |
| | | cggcctaccaaggcgacgatcagtagctggtctgagaggatgatc |
| | | agccacactgggactgagacacggcccagactcctacgggaggca |
| | | gcagtggggaatattggacaatgggcgcaagcctgatccagccat |
| | | gccgcgtgagtgatgaaggccttagggttgtaaagctcttttgtc |
| | | cgggacgataatgacggtaccggaagaataagccccggctaactt |
| | | cgtgccagcagccgcggtaatacgaagggggctagcgttgctcgg |
| | | aatcactgggcgtaaagggcgcgtaggcggccgattaagtcgggg |
| | | gtgaaagcctgtggctcaaccacagaattgccttcgatactggtt |
| | | ggcttgagaccggaagaggacagcggaactgcgagtgtagaggtg |
| | | aaattcgtagatattcgcaagaacaccagtggcgaaggcggctgt |
| | | ctggtccggttctgacgctgaggcgcgaaagcgtggggagcaaac |
| | | aggattagataccctggtagtccacgccgtaaacgatgaatgcca |
| | | gccgttggtctgcttgcaggtcagtggcgccgctaacgcattaag |
| | | cattccgcctggggagtacggtcgcaagattaaaactcaaaggaa |
| | | ttgacgggggcccgcacaagcggtggagcatgtggtttaattcga |
| | | agcaacgcgcagaaccttaccatcccttgacatggcatgttacct |
| | | cgagagatcggggatcctcttcggaggcgtgcacacaggtgctgc |
| | | atggctgtcgtcagctcgtgtcgtgagatgttgggttaagtcccg |
| | | caacgagcgcaacccacgtccttagttgccatcattcagttgggc |
| | | actctagggagactgccggtgataagccgcgaggaaggtgtggat |
| | | gacgtcaagtcctcatggcccttacgggatgggctacacacgtgc |
| | | tacaatggcggtgacagtgggacgcgaaaccgcgaggttgagcaa |
| | | atccccaaaagccgtctcagttcggattgcactctgcaactcggg |
| | | tgcatgaaggcggaatcgctagtaatcgtggatcagcacgccacg |
| | | gtgaatacgttcccgggccttgtacacaccgcccgtcacaccatg |
| | | ggagttggtcttacccgacggcgctgcgccaaccgcaagggggca |
| | | ggcgaccacggtagggtcagcgactggggtgaagtcgtaacaagg |
| | | tagccgtaggggaacctgcggctggatcacct |
| NLS0109 | 8 | ggtgatccagccgcaggttcccctacggctaccttgttacgactt |
| | | caccccagtcgctgaccctaccgtggtcgcctgctccccttgcgg |
| | | gtcggcgcagcgccgtcgggtaagaccaactcccatggtgtgacg |
| | | ggcggtgtgtacaaggcccgggaacgtattcaccgtggcatgctg |
| | | atccacgattactagcgattccgccttcatgcactcgagttgcag |
| | | agtgcaatccgaactgagacggcttttggagatttgcttgccctc |
| | | gcgggttcgcgtcccactgtcaccgccattgtagcacgtgtgtag |
| | | cccatcccgtaagggccatgaggacttgacgtcatccacaccttc |
| | | ctcgcggcttatcaccggcagtctccccagagtgcccaactgaat |
| | | gatggcaactgaggacgtgggttgcgctcgttgcgggacttaacc |
| | | caacatctcacgacacgagctgacgacagccatgcagcacctgtg |
| | | tgcgcgctcccgaaggagaccgtggatctctccacgtaacacgcc |
| | | atgtcaaaggatggtaaggttctgcgcgttgcttcgaattaaacc |
| | | acatgctccaccgcttgtgcgggcccccgtcaattcctttgagtt |
| | | ttaatcttgcgaccgtactccccaggcggaatgctcaaagcgtta |
| | | gctgcgccactgagaggcaagccccccaacggctggcattcatcg |
| | | tttacggcgtggactaccagggtatctaatcctgtttgctcccca |
| | | cgctttcgcgcctcagcgtcagtgtcggaccagttggccgccttc |
| | | gccaccggtgttcttgcgaatatctacgaatttcacctctacact |
| | | cgcagttccaccaacctcttccgaactcaagtctcccagtatcga |
| | | aggcaattctgtggttgagccacaggctttcacccccgacttaaa |
| | | agaccgcctacgcgccctttacgcccagtgattccgagcaacgct |
| | | agcccccttcgtattaccgcggctgctggcacgaagttagccggg |
| | | gcttattcctccggtaccgtcattatcgtcccggataaaagagct |
| | | ttacaaccctaaggccttcatcactcacgcggcatggctggatca |
| | | ggcttgcgcccattgtccaatattccccactgctgcctcccgtag |
| | | gagtctgggccgtgtctcagtcccagtgtggctgatcatcctctc |
| | | agaccagctactgatcgtcgccttggtaggccgttaccccaccaa |
| | | ctagctaatcagacgcgggccgatcttccggcagtaaacctttcc |
| | | ccaaaagggcgtatccggtattagctcaagtttccctgagttatt |
| | | ccgaaccagaaggcacgttcccacgcgttactcacccgtccgccg |
| | | ctgacaccgaagtgcccgctcgacttgcatgtgttaagcctgccg |
| | | ccagcgttcgctctgagccaggatcaaactctc |

Also provided are methods of making the compositions useful for controlling plant pathogenic fungi that comprise combining a Methylobacterium that inhibit growth of a plant pathogenic fungus with an agriculturally acceptable excipient and/or with an agriculturally acceptable adjuvant. In certain embodiments of the methods, the Methylobacterium is not M. radiotolerans or M. oryzae. In certain embodiments of any of the aforementioned methods, the composition comprises a Methylobacterium sp. related to NLS0109 that has; (i) at least one gene encoding a 16S RNA that has at least 97%, 98%, 99%, 99.5%, or 100% sequence identity to SEQ ID NO: 8; (ii) in its genome one or more polynucleotide marker fragments of at least 50, 60, 100, 120, 180, 200, 240, or 300 nucleotides of SEQ ID NOS: 9-11; or (iii) in its genome one or more marker fragments comprising a sequence having at least 98%, 99%, or 99.5% sequence identity across the entire length of SEQ ID NOS: 9-11. In certain embodiments of any of the aforementioned methods, the composition comprises the Methylobacterium isolate NLS0109, a derivative thereof, or a Methylobacterium related to NLS0109. In certain embodiments of any of the aforementioned methods, the composition further comprises Methylobacterium strain

NLS0017, NLS0020, NLS0021, NLS0042, NLS0064, NLS0066, NLS0089, a derivative thereof, or a Methylobacterium sp. related thereto. In certain embodiments of the methods, the compositions provide for at least about 25%, at least about 50%, or at least about 75% inhibition of plant pathogenic fungal growth in comparison to a control composition that lacks Methylobacterium that inhibit a plant pathogenic fungus upon exposure to the plant pathogenic fungus. In certain embodiments of the methods, the plant pathogenic fungus is selected from the group consisting of a Blumeria sp., a Cercospora sp., a Cochliobolus sp., a Colletotrichum sp., a Diplodia sp., an Exserohilum sp., a Fusarium sp., a Gacumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. and a Verticillium sp.

In certain embodiments of the methods, the Fusarium sp. is selected from the group consisting of Fusarium graminearum, Fusarium verticillioides, Fusarium oxysporum, and Fusarium solani. In certain embodiments of the methods, the Methylobacterium is adhered to a solid substance. In certain embodiments of the methods, the Methylobacterium adhered to the solid substance is combined with a liquid to form a composition that is a colloid. In certain embodiments of the methods, the colloid is a gel. In certain embodiments of the methods, the Methylobacterium adhered to the solid substance is provided by culturing the Methylobacterium in the presence of the solid substance. In certain embodiments of the methods, the composition comprises an emulsion. In certain embodiments of the methods, the Methylobacterium is provided by culturing the Methylobacterium in an emulsion. In certain embodiments of any of the aforementioned methods, the plant pathogenic fungus is a Fusarium sp. and/or the plant is a cereal plant. In certain embodiments of any of the aforementioned methods, the plant pathogenic fungus is a Fusarium sp. and the plant is a cereal plant selected from the group consisting of a rice, wheat, corn, barley, millet, sorghum, oat, and rye plant. In certain embodiments of any of the aforementioned methods, the plant pathogenic fungus is Fusarium graminearum and the plant is a cereal plant selected from the group consisting of a rice, wheat, corn, barley, millet, sorghum, oat, and rye plant. In any of the aforementioned embodiments, the plant pathogenic fungi that is inhibited can be in its anamorphic form, its teleomorphic form, or in both its anamorphic and telcomorphic forms.

Methods where Methylobacterium are cultured in biphasic media comprising a liquid phase and a solid substance have been found to significantly increase the resultant yield of Methylobacterium relative to methods where the Methylobacterium are cultured in liquid media alone. In certain embodiments, the methods can comprise growing the Methylobacterium in liquid media with a particulate solid substance that can be suspended in the liquid by agitation under conditions that provide for Methylobacterium growth. In certain embodiments where particulate solid substances are used, at least substantially all of the solid phase can thus be suspended in the liquid phase upon agitation. Such particulate solid substances can comprise materials that are about 1 millimeter or less in length or diameter. In certain embodiments, the degree of agitation is sufficient to provide for uniform distribution of the particulate solid substance in the liquid phase and/or optimal levels of culture aeration. However, in other embodiments provided herein, at least substantially all of the solid phase is not suspended in the liquid phase, or portions of the solid phase are suspended in the liquid phase and portions of the solid phase are not suspended in the liquid phase. Non-particulate solid substances can be used in certain biphasic media where the solid phase is not suspended in the liquid phase. Such non-particulate solid substances include, but are not limited to, materials that are greater than about 1 millimeter in length or diameter. Such particulate and non-particulate solid substances also include, but are not limited to, materials that are porous, fibrous, or otherwise configured to provide for increased surface areas for adherent growth of the Methylobacterium. Biphasic media where portions of the solid phase are suspended in the liquid phase and portions of the solid phase are not suspended in the liquid phase can comprise a mixture of particulate and non-particulate solid substances. Such particulate and non-particulate solid substances used in any of the aforementioned biphasic media also include, but are not limited to, materials that are porous, fibrous, or otherwise configured to provide for increased surface areas for adherent growth of the Methylobacterium. In certain embodiments, the media comprises a colloid formed by a solid and a liquid phase. A colloid comprising a solid and a liquid can be pre-formed and added to liquid media or can be formed in media containing a solid and a liquid. Colloids comprising a solid and a liquid can be formed by subjecting certain solid substances to a chemical and/or thermal change. In certain embodiments, the colloid is a gel. In certain embodiments, the liquid phase of the media is an emulsion. In certain embodiments, the emulsion comprises an aqueous liquid and a liquid that is not miscible, or only partially miscible, in the aqueous liquid. Liquids that are not miscible, or only partially miscible, in water include, but are not limited to, any of the following: (1) liquids having a miscibility in water that is equal to or less than that of pentanol, hexanol, or heptanol at 25 degrees C.; (2) liquids comprising an alcohol, an aldehyde, a ketone, a fatty acid, a phospholipid, or any combination thereof; (3) alcohols selected from the group consisting of aliphatic alcohols containing at least 5 carbons and sterols; (4) an animal oil, microbial oil, synthetic oil, plant oil, or combination thereof; and/or, (5) a plant oil selected from the group consisting of corn, soybean, cotton, peanut, sunflower, olive, flax, coconut, palm, rapeseed, sesame seed, safflower, and combinations thereof. In certain embodiments, the immiscible or partially immiscible liquid can comprise at least about 0.02% to about 20% of the liquid phase by mass. In certain embodiments, the methods can comprise obtaining a biphasic culture media comprising the liquid, the solid, and Methylobacterium and incubating the culture under conditions that provide for growth of the Methylobacterium. Biphasic culture medias comprising the liquid, the solid, and Methylobacterium can be obtained by a variety of methods that include, but are not limited to, any of: (a) inoculating a biphasic media comprising the liquid and the solid substance with Methylobacterium; (b) inoculating the solid substance with Methylobacterium and then introducing the solid substance comprising the Methylobacterium into the liquid media; (c) inoculating the solid substance with Methylobacterium, incubating the Methylobacterium on the solid substance, and then introducing the solid substance comprising the Methylobacterium into the liquid media; or (d) any combination of (a), (b), or (c). Methods and compositions for growing Methylobacterium in biphasic media comprising a liquid and a solid are disclosed in co-assigned US Patent Application Publication No. 20130324407, which is incorporated herein by reference in its entirety.

Methods where Methylobacterium are cultured in media comprising an emulsion have also been found to significantly increase the resultant yield of Methylobacterium relative to methods where the Methylobacterium are cultured in liquid media alone. In certain embodiments, the methods for making the compositions provided herein can comprise growing the Methylobacterium in an emulsion under conditions that provide for Methylobacterium growth. Medias comprising the emulsion and Methylobacterium can be obtained by a variety of methods that include, but are not limited to, any of: (a) inoculating a media comprising the emulsion with Methylobacterium; (b) inoculating the aqueous liquid with the Methylobacterium, introducing the non-aqueous liquid, and mixing to form an emulsion; (c) inoculating the aqueous liquid with the Methylobacterium, introducing the non-aqueous liquid, and mixing to form an emulsion; or (d) any combination of (a), (b), or (c). In certain embodiments, the emulsion comprises an aqueous liquid and a liquid that is not miscible, or only partially miscible, in the aqueous liquid. Non-aqueous liquids that are not miscible, or only partially miscible, in water include, but are not limited to, any of the following: (1) liquids having a miscibility in water that is equal to or less than that of n-pentanol, n-hexanol, or n-heptanol at 25 degrees C.; (2) liquids comprising an alcohol, an aldehyde, a ketone, a fatty acid, a phospholipid, or any combination thereof; (3) alcohols selected from the group consisting of aliphatic alcohols containing at least 5, 6, or 7 carbons and sterols; (4) an animal oil, microbial oil, synthetic oil, plant oil, or combination thereof; and/or, (5) a plant oil selected from the group consisting of corn, soybean, cotton, peanut, sunflower, olive, flax, coconut, palm, rapeseed, sesame seed, safflower, and combinations thereof. In certain embodiments, the immiscible or partially immiscible non-aqueous liquid can comprise at least about 0.02% to about 20% of the emulsion by mass. In certain embodiments, the immiscible or partially immiscible non-aqueous liquid can comprise at least about any of about 0.05%, 0.1%, 0.5%, or 1% to about 3%, 5%, 10%, or 20% of the emulsion by mass. Methods and compositions for growing Methylobacterium in media comprising an emulsion are disclosed in co-assigned U.S. patent application Ser. No. 14/894,568, filed May 30, 2014, which is incorporated herein by reference in its entirety.

In certain embodiments, the fermentation broth, fermentation broth product, or compositions that comprise Methylobacterium that inhibit plant pathogenic fungi can further comprise one or more introduced microorganisms of pre-determined identity other than Methylobacterium. Other microorganisms that can be added include, but are not limited to, microorganisms that are biopesticidal or provide some other benefit when applied to a plant or plant part. Biopesticidal or otherwise beneficial microorganisms thus include, but are not limited to, various Bacillus sp., Pseudomonas sp., Coniothyrium sp., Pantoea sp., Streptomyces sp., and Trichoderma sp., as well as bioinoculants for improved nitrogen fixation, including rhizobia species such as from Rhizobium or Bradyrhizobium, Microbial biopesticides can be a bacterium, fungus, virus, or protozoan. Particularly useful biopesticidal microorganisms include various Bacillus subtilis, Bacillus thuringiensis, Bacillus pumilis, Pseudomonas syringae, Trichoderma harzianum, Trichoderma virens, and Streptomyces lydicus strains. Other microorganisms that are added can be genetically engineered or isolates that are available as pure cultures. In certain embodiments, it is anticipated that the bacterial or fungal microorganism can be provided in the fermentation broth, fermentation broth product, or composition in the form of a sporc.

In certain embodiments, the liquid culture medium is prepared from inexpensive and readily available components, including, but not limited to, inorganic salts such as potassium phosphate, magnesium sulfate and the like, carbon sources such as glycerol, methanol, glutamic acid, aspartic acid, succinic acid and the like, and amino acid blends such as peptone, tryptonc, and the like. Examples of liquid media that can be used include, but are not limited to, ammonium mineral salts (AMS) medium (Whittenbury et al., 1970), Vogel-Bonner (VB) minimal culture medium (Vogel and Bonner, 1956), and LB broth (“Luria-Bertani Broth”).

Fermentation products and compositions with a mono- or co-culture of Methylobacterium that inhibit plant pathogenic fungi at a titer of greater than about 5×10⁷ colony-forming units per milliliter, at a titer of greater than about 1×10⁸ colony-forming units per milliliter, at a titer of greater than about 5×10⁸ colony-forming units per milliliter, at a titer of greater than about 1×10⁹ colony-forming units per milliliter, at a titer of greater than about 1×10¹⁰ colony-forming units per milliliter, at a titer of at least about 3×10¹⁰ colony-forming units per milliliter are provided herein. In certain embodiments, fermentation products and compositions provided herein can comprise Methylobacterium that inhibit plant pathogenic fungi at a titer of at least about 5×10⁷, 1×10⁸, or 5×10⁸ colony-forming units per milliliter to at least about 3×10¹⁰ colony-forming units per milliliter, at least about 5×10⁸ colony-forming units per milliliter to at least about 4×10¹⁰ colony-forming units per milliliter, or at least about 5×10⁸ colony-forming units per milliliter to at least about 6×10¹⁰ colony-forming units per milliliter. In certain embodiments, fermentation products and compositions provided herein can comprise Methylobacterium that inhibit plant pathogenic fungi at a titer of at least about 1×10⁹ colony-forming units per milliliter to at least about 3×10¹⁰ colony-forming units per milliliter, at least about 1×10⁹ colony-forming units per milliliter to at least about 4×10¹⁰ colony-forming units per milliliter, or at least about 1×10⁹ colony-forming units per milliliter to at least about 6×10¹⁰ colony-forming units per milliliter. In certain embodiments, fermentation products and compositions provided herein will comprise Methylobacterium that inhibit plant pathogenic fungi at a titer of at least about 1×10¹⁰ colony-forming units per milliliter to at least about 3×10¹⁰ colony-forming units per milliliter, at least about 1×10¹⁰ colony-forming units per milliliter to at least about 4×10¹⁰ colony-forming units per milliliter, or at least about 1×10¹⁰ colony-forming units per milliliter to at least about 6×10¹⁰ colony-forming units per milliliter. In certain embodiments, fermentation products and compositions provided herein will comprise Methylobacterium that inhibit plant pathogenic fungi at a titer of, at least about 3×10¹⁰ colony-forming units per milliliter to at least about 4×10¹⁰ colony-forming units per milliliter, or at least about 3×10¹⁰ colony-forming units per milliliter to at least about 6×10¹⁰ colony-forming units per milliliter. In any of the aforementioned fermentation products or compositions, the indicated concentrations can be fungal inhibitory concentrations. In any of the aforementioned fermentation products or compositions, the fermentation products or compositions can be essentially free of contaminating microorganisms, can comprise Methylobacterium that are adhered to and/or associated with materials that the Methylobacterium are not are adhered to and/or associated with in nature, or any combination thereof.

Fermentation products and compositions with Methylobacterium that inhibit plant pathogenic fungi at a titer of greater than about 5×10⁷, 1×10⁸, or 5×10⁸ colony-forming units per gram, at a titer of greater than about 1×10¹⁰ colony-forming units per gram, at a titer of greater than about 1×10¹⁰ colony-forming units per gram, at a titer of at least about 3×10¹⁰ colony-forming units per gram are provided herein. In certain embodiments, fermentation products and compositions provided herein can comprise Methylobacterium that inhibit plant pathogenic fungi at a titer of at least about 5×10⁷, 1×10⁸, or 5×10⁸ colony-forming units per gram to at least about 3×10¹⁰ colony-forming units per gram, at least about 5×10⁷, 1×10⁸, or 5×10⁸ colony-forming units per gram to at least about 4×10¹⁰ colony-forming units per gram, or at least about 5×10⁷, 1×10⁸, or 5×10⁸ colony-forming units per gram to at least about 6×10¹⁰ colony-forming units per gram. In certain embodiments, fermentation products and compositions provided herein can comprise Methylobacterium that inhibit plant pathogenic fungi at a titer of at least about 1×10⁹ colony-forming units per gram to at least about 3×10¹⁰ colony-forming units per gram, at least about 1×10⁹ colony-forming units per gram to at least about 4×10¹⁰ colony-forming units per gram, or at least about 1×10⁹ colony-forming units per gram to at least about 6×10¹⁰ colony-forming units per gram. In certain embodiments, fermentation products and compositions provided herein will comprise Methylobacterium that inhibit plant pathogenic fungi at a titer of at least about 1×10¹⁰ colony-forming units per gram to at least about 3×10¹⁰ colony-forming units per gram, at least about 1×10¹⁰ colony-forming units per gram to at least about 4×10¹⁰ colony-forming units per gram, or at least about 1×10¹⁰ colony-forming units per gram to at least about 6×10¹⁰ colony-forming units per gram. In certain embodiments, fermentation products and compositions provided herein will comprise Methylobacterium that inhibit plant pathogenic fungi at a titer of, at least about 3×10¹⁰ colony-forming units per gram to at least about 4×10¹⁰ colony-forming units per gram, or at least about 3×10¹⁰ colony-forming units per gram to at least about 6×10¹⁰, 1×10¹¹, 1×10¹², 1×10¹³, or 5×10¹³ colony-forming units per gram. In any of the aforementioned fermentation products or compositions, the fermentation or composition can comprise a mono- or co-culture of Methylobacterium that is adhered to a solid substance. In any of the aforementioned fermentation products or compositions, the indicated concentrations can be fungal inhibitory concentrations. In any of the aforementioned fermentation products or compositions, the indicated concentrations can be fungal inhibitory concentrations. In any of the aforementioned fermentation products or compositions, the fermentation products or compositions can be essentially free of contaminating microorganisms, can comprise Methylobacterium that are adhered to and/or associated with materials that the Methylobacterium are not are adhered to and/or associated with in nature, or any combination thereof.

Methylobacterium that inhibit plant pathogenic fungi can be obtained as fermentation products can be used to make various compositions useful for treating plants or plant parts to inhibit infection by plant pathogenic fungi. Alternatively, compositions provided herein comprising solid substances with Methylobacterium that inhibit plant pathogenic fungi or adherent Methylobacterium that inhibit plant pathogenic fungi can be used to treat plants or plant parts. Plants, plant parts, and, in particular, plant seeds that have been at least partially coated with the fermentation broth products or compositions comprising Methylobacterium that inhibit plant pathogenic fungi are thus provided. Also provided are processed plant products that contain the fermentation broth products or compositions with Methylobacterium that inhibit plant pathogenic fungi or adherent Methylobacterium that inhibit plant pathogenic fungi. Methylobacterium that inhibit plant pathogenic fungi can be used to make various compositions that are particularly useful for treating plant seeds. Seeds that have been at least partially coated with the fermentation broth products or compositions are thus provided. Also provided are processed seed products, including, but not limited to, meal, flour, feed, and flakes that contain the fermentation broth products or compositions provided herein. In certain embodiments, the processed plant product will be non-regenerable (i.e. will be incapable of developing into a plant). In certain embodiments, the fermentation product or composition that at least partially coats the plant, plant part, or plant seed or that is contained in the processed plant, plant part, or seed product comprises a Methylobacterium that inhibit plant pathogenic fungi that can be readily identified by comparing a treated and an untreated plant, plant part, plant seed, or processed product thereof. In certain embodiments, the identification is performed by determining if the fermentation product or composition that at least partially coats the plant, plant part, or plant seed or that is contained in the processed plant, plant part, or seed product comprises a Methylobacterium sp. related to NLS0109 that: (i) has at least one gene encoding a 16S RNA that has at least 97%, 98%, 99%, 99.5%, or 100% sequence identity to SEQ ID NO: 8; (ii) has in its genome one or more polynucleotide marker fragments of at least 50, 60, 100, 120 , 180, 200, 240, or 300 nucleotides of SEQ ID NOS: 9-11; or (iii) has in its genome one or more marker fragments comprising a sequence having at least 98%, 99%, or 99.5% sequence identity across the entire length of SEQ ID NOS: 9-11. Similar 16S RNA sequence analyses can be conducted to determine if the fermentation product or composition that at least partially coats the plant, plant part, or plant seed or that is contained in the processed plant, plant part, or seed product further comprises NLS0017, NLS0020, NLS0021, NLS0042, NLS0064, NLS0066, NLS0089, a derivative thereof, or a Methylobacterium sp. related thereto.

Compositions useful for treating plants or plant parts that comprise Methylobacterium that inhibit plant pathogenic fungi or a solid substance with adherent Methylobacterium that inhibit plant pathogenic fungi, emulsions containing the Methylobacterium that inhibit plant pathogenic fungi or combinations thereof can also comprise an agriculturally acceptable adjuvant or an agriculturally acceptable excipient. An agriculturally acceptable adjuvant or an agriculturally acceptable excipient is typically an ingredient that does not cause undue phytotoxicity or other adverse effects when exposed to a plant or plant part. In certain embodiments, the solid substance can itself be an agriculturally acceptable adjuvant or an agriculturally acceptable excipient so long as it is not bacteriocidal or bacteriostatic to the Methylobacterium. In other embodiments, the composition further comprises at least one of an agriculturally acceptable adjuvant or an agriculturally acceptable excipient. Any of the aforementioned compositions can also further comprise a pesticide. Pesticides used in the composition include, but are not limited to, an insecticide, a fungicide, a nematocide, and a bacteriocide. In certain embodiments, the pesticide used in the composition is a pesticide that does not substantially inhibit growth of the Methylobacterium. As Methylobacterium are gram negative bacteria, suitable bacteriocides used in the compositions can include, but are not limited to, bacteriocides that exhibit activity against gram positive bacteria but not gram negative bacteria. Compositions provided herein can also comprise a bacteriostatic agent that does not substantially inhibit growth of the Methylobacterium. Bacteriostatic agents suitable for use in compositions provided herein include, but are not limited to, those that exhibit activity against gram positive bacteria but not gram negative bacteria. Any of the aforementioned compositions can also be an essentially dry product (i.e. having about 5% or less water content), a mixture of the composition with an emulsion, or a suspension.

Agriculturally acceptable adjuvants used in the compositions that comprise Methylobacterium that inhibit plant pathogenic fungi, emulsions containing the Methylobacterium that inhibit plant pathogenic fungi, or combinations thereof include, but are not limited to, components that enhance product efficacy and/or products that enhance case of product application. Adjuvants that enhance product efficacy can include various wetters/spreaders that promote adhesion to and spreading of the composition on plant parts, stickers that promote adhesion to the plant part, penetrants, extenders, and humectants that increase the density or drying time of sprayed compositions. Wetters/spreaders used in the compositions can include, but are not limited to, non-ionic surfactants, anionic surfactants, cationic surfactants, amphoteric surfactants, organo-silicate surfactants, and/or acidified surfactants. Stickers used in the compositions can include, but are not limited to, latex-based substances, terpene/pinolene, and pyrrolidone-based substances. Penetrants can include mineral oil, vegetable oil, esterified vegetable oil, organo-silicate surfactants, and acidified surfactants. Extenders used in the compositions can include, but are not limited to, ammonium sulphate, or menthene-based substances. Humectants used in the compositions can include, but are not limited to, glycerol, propylene glycol, and diethyl glycol. Adjuvants that improve case of product application include, but are not limited to, acidifying/buffering agents, anti-foaming/de-foaming agents, compatibility agents, drift-reducing agents, dyes, and water conditioners. Anti-foaming/de-foaming agents used in the compositions can include, but are not limited to, dimethopolysiloxane. Compatibility agents used in the compositions can include, but are not limited to, ammonium sulphate. Drift-reducing agents used in the compositions can include, but are not limited to, polyacrylamides, and polysaccharides. Water conditioners used in the compositions can include, but are not limited to, ammonium sulphate.

Methods of treating plants and/or plant parts with the fermentation broths, fermentation broth products, and compositions comprising Methylobacterium that inhibit plant pathogenic fungi, or combinations thereof are also provided herein. Treated plants, and treated plant parts obtained therefrom, include, but are not limited to, corn, Brassica sp. (e.g., B. napus, B. rapa, B. juncea), alfalfa, rice, rye, sorghum, millet (e.g., pearl millet (Pennisetum glaucum)), proso millet (Panicum miliaceum), foxtail millet (Setaria italica), finger millet (Eleusine coracana), sunflower, safflower, soybean, tobacco, potato, peanuts, cotton, sweet potato (Ipomoea batatus), cassava, coffee, coconut, pineapple, citrus trees, cocoa, tea, banana, avocado, fig, guava, mango, olive, papaya, cashew, macadamia, almond, sugar beets, sugarcane, oats, barley, tomatoes, lettuce, green beans, lima beans, peas, cucurbits such as cucumber, cantaloupe, and musk melon, ornamentals, and conifers. Plant parts that are treated include, but are not limited to, leaves, stems, flowers, roots, seeds, fruit, tubers, coleoptiles, and the like. Ornamental plants and plant parts that can be treated include, but are not limited to azalea, hydrangea, hibiscus, roses, tulips, daffodils, petunias, carnation, poinsettia, and chrysanthemum. Conifer plants and plant parts that can be treated include, but are not limited to, pines such as loblolly pine, slash pine, ponderosa pine, lodge pole pine, and Monterey pine; Douglas-fir; Western hemlock; Sitka spruce; redwood; true firs such as silver fir and balsam fir; and cedars such as Western red cedar and Alaska yellow-cedar. Turfgrass plants and plant parts that can be treated include, but are not limited to, annual bluegrass, annual ryegrass, Canada bluegrass, fescue, bentgrass, wheatgrass, Kentucky bluegrass, orchard grass, ryegrass, redtop, Bermuda grass, St. Augustine grass, and zoysia grass. In certain embodiments, the treated plant or plant part is a cereal plant or plant part selected from the group consisting of a rice, wheat, corn, barley, millet, sorghum, oat, and rye plant or plant part. Seeds or other propagules of any of the aforementioned plants can be treated with the fermentation broths, fermentation broth products, fermentation products, and/or compositions provided herein.

In certain embodiments, plants and/or plant parts are treated by applying the fermentation broths, fermentation broth products, fermentation products, and compositions that comprise Methylobacterium that inhibit plant pathogenic fungi, or combinations thereof as a spray. Such spray applications include, but are not limited to, treatments of a single plant part or any combination of plant parts. Spraying can be achieved with any device that will distribute the fermentation broths, fermentation broth products, fermentation products, and compositions to the plant and/or plant part(s). Useful spray devices include a boom sprayer, a hand or backpack sprayer, crop dusters (i.e. aerial spraying), and the like. Spraying devices and or methods providing for application of the fermentation broths, fermentation broth products, fermentation products, and compositions to either one or both of the adaxial surface and/or abaxial surface can also be used. Plants and/or plant parts that are at least partially coated with any of a biphasic fermentation broth, a fermentation broth product, fermentation product, or compositions that comprise a solid substance with Methylobacterium that inhibit plant pathogenic fungi adhered thereto are also provided herein. Also provided herein are processed plant products that comprise a solid substance with Methylobacterium that inhibit plant pathogenic fungi adhered thereto.

In certain embodiments, seeds are treated by exposing the seeds to the fermentation broths, fermentation broth products, fermentation products, and compositions that comprise Methylobacterium that inhibit plant pathogenic fungi, or combinations thereof. Seeds can be treated with the fermentation broths, fermentation broth products, and compositions provided herein by methods including, but not limited to, imbibition, coating, spraying, and the like. Seed treatments can be effected with both continuous and/or a batch seed treaters. In certain embodiments, the coated seeds can be prepared by slurrying seeds with a coating composition containing a fermentation broth, fermentation broth product, or compositions that comprise the solid substance with Methylobacterium that inhibit plant pathogenic fungi and air drying the resulting product. Air drying can be accomplished at any temperature that is not deleterious to the seed or the Methylobacterium, but will typically not be greater than 30 degrees Centigrade. The proportion of coating that comprises a solid substance and Methylobacterium that inhibit plant pathogenic fungi includes, but is not limited to, a range of 0.1 to 25% by weight of the seed, 0.5 to 5% by weight of the seed, and 0.5 to 2.5% by weight of seed. In certain embodiments, a solid substance used in the seed coating or treatment will have Methylobacterium that inhibit plant pathogenic fungi adhered thereon. In certain embodiments, a solid substance used in the seed coating or treatment will be associated with Methylobacterium that inhibit plant pathogenic fungi and will be a fermentation broth, fermentation broth product, or composition obtained by the methods provided herein. Various seed treatment compositions and methods for seed treatment disclosed in U.S. Pat. Nos. 5,106,648; 5,512,069; and 8,181,388 are incorporated herein by reference in their entireties and can be adapted for use with fermentation products or compositions provided herein. In certain embodiments, the composition used to treat the seed can contain agriculturally acceptable excipients that include, but are not limited to, woodflours, clays, activated carbon, diatomaceous earth, fine-grain inorganic solids, calcium carbonate and the like. Clays and inorganic solids that can be used with the fermentation broths, fermentation broth products, or compositions provided herein include, but are not limited to, calcium bentonite, kaolin, china clay, talc, perlite, mica, vermiculite, silicas, quartz powder, montmorillonite and mixtures thereof. Agriculturally acceptable adjuvants that promote sticking to the seed that can be used include, but are not limited to, polyvinyl acetates, polyvinyl acetate copolymers, hydrolyzed polyvinyl acetates, polyvinylpyrrolidone-vinyl acetate copolymer, polyvinyl alcohols, polyvinyl alcohol copolymers, polyvinyl methyl ether, polyvinyl methyl ether-maleic anhydride copolymer, waxes, latex polymers, celluloses including ethylcelluloses and methylcelluloses, hydroxy methylcelluloses, hydroxypropylcellulose, hydroxymethylpropylcelluloses, polyvinyl pyrrolidones, alginates, dextrins, malto-dextrins, polysaccharides, fats, oils, proteins, karaya gum, jaguar gum, tragacanth gum, polysaccharide gums, mucilage, gum arabics, shellacs, vinylidene chloride polymers and copolymers, soybean-based protein polymers and copolymers, lignosulfonates, acrylic copolymers, starches, polyvinylacrylates, zeins, gelatin, carboxymethylcellulose, chitosan, polyethylene oxide, acrylamide polymers and copolymers, polyhydroxyethyl acrylate, methylacrylamide monomers, alginate, ethylcellulose, polychloroprene and syrups or mixtures thereof. Other useful agriculturally acceptable adjuvants that can promote coating include, but are not limited to, polymers and copolymers of vinyl acetate, polyvinylpyrrolidone-vinyl acetate copolymer and water-soluble waxes. Various surfactants, dispersants, anticaking-agents, foam-control agents, and dyes disclosed herein and in U.S. Pat. No. 8,181,388 can be adapted for use with a fermentation products or compositions provided herein.

Provided herein are compositions that comprise Methylobacterium that inhibit plant pathogenic fungi and that provide control of plant pathogenic fungal infections of plants, plant parts, and plants obtained therefrom relative to untreated plants, plant parts, and plants obtained therefrom that have not been exposed to the compositions. In certain embodiments, plant parts, including, but not limited to, a seed, a leaf, a fruit, a stem, a root, a tuber, or a coleoptile can be treated with the compositions provided herein to control fungal disease. Treatments or applications can include, but are not limited to, spraying, coating, partially coating, immersing, and/or imbibing the plant or plant parts with the compositions provided herein. In certain embodiments, a seed, a leaf, a fruit, a stem, a root, a tuber, or a coleoptile can be immersed and/or imbibed with a liquid, semi-liquid, emulsion, or slurry of a composition provided herein. Such seed immersion or imbibition can be sufficient to provide for fungal disease inhibition in a plant or plant part in comparison to an untreated plant or plant part. Such fungal disease inhibition includes, but is not limited to decreases in fungal growth and/or the adverse effects of fungal growth relative to untreated plants. In certain embodiments, plant seeds can be immersed and/or imbibed for at least 1, 2, 3, 4, 5, or 6 hours. Such immersion and/or imbibition can, in certain embodiments, be conducted at temperatures that are not deleterious to the plant seed or the Methylobacterium. In certain embodiments, the seeds can be treated at about 15 to about 30degrees Centigrade or at about 20 to about 25 degrees Centigrade. In certain embodiments, seed imbibition and/or immersion can be performed with gentle agitation.

Amounts of the compositions that comprise Methylobacterium that inhibit plant pathogenic fungi that are sufficient to provide for an inhibition of fungal infection of a plant or plant part can thus be determined by measuring any or all fungal growth and/or the adverse effects of fungal growth in treated plants or plant parts relative to untreated plants or plant parts. Adverse effects of fungal growth in a plant that can be measured include any type of plant tissue damage or necrosis, any type of plant yield reduction, any reduction in the value of the crop plant product, and/or production of undesirable fungal metabolites or fungal growth by-products including, but not limited to, mycotoxins. Mycotoxins comprise a number of toxic molecules produced by fungal species, including, but not limited to, polyketides (including aflatoxins, demethylsterigmatocystin, O-methylsterigmatocystin etc.), fumonisins, alperisins (e.g., A1, A2, B1, B2), sphingofungins (A, B, C and D), trichothecenes, fumifungins, and the like. Methods of quantitating mycotoxin levels are widely documented. Moreover, commercial kits for measurement of the mycotoxins such as aflatoxin, fumonisin, deoxynivalenol, and zearalenone are also available (VICAM, Watertown, MA, USA).

Compositions provided herein comprising Methylobacterium that inhibit plant pathogenic fungi are therefore expected to be useful in inhibiting fungal growth and/or infection in a wide variety of plant pathogenic fungi, including, but not limited to the anamorphic and/or telcomorphic stages of those phytopathogenic fungi in the following genera and species: Blumeria (Blumeria graminis f. sp. tritici), Cercospora (Cercospora kikuchii; Cercospora sojina; Cercospora zeae-maydis); Cochliobolus (Colchliobolus maydis; Cochliobolus heterostrophus; Cochliobolus carbonum); Colletotrichum (Colletotrichum lindemuthianum; Colletotrichum graminicola; Colletotrichum cereale); Diplodia (Diplodia maydis); Exserohilum(Exserohilum turcicum); Fusarium (Fusarium nivale; Fusarium oxysporum; Fusarium graminearum; Fusarium culmorum; Fusarium solani; Fusarium moniliforme; Fusarium virguliforme); Macrophomina (Macrophomina phaseolina); Magnaporthe (Magnaporthe oryzae; Magnaporthe grisea); Phakopsora (Phakopsora pachyrhizi); Phialopora (Phialophora gregata); Phymatotrichum (Phymatotrichum omnivorum); Phytophthora (Phytophthora cinnamomi; Phytophthora cactorum; Phytophthora phaseoli; Phytophthora parasitica; Phytophthora citrophthora; Phytophthora megasperma f.sp. sojae; Phytophthora infestans); Puccinia(Puccinia sorghi; Puccinia striiformis; Puccinia graminis f.sp. tritici; Puccinia asparagi; Puccinia recondita; Puccinia arachidis; Puccinia coronata); Pythium (Pythium aphanidermatum; Pythium ultimum; Pythium sylvaticum; Pythium torulosum; Pythium lutarium; Pythium oopapillum); Pyrenophora (Pyrenophora tritici-repentis); Rhizoctonia (Rhizoctonia solani; Rhizoctonia cerealis); Sclerotium (Sclerotium rolfsii); Sclerotinia (Sclerotinia sclerotiorum; Sclerotinia homoeocarpa); Septoria (Septoria lycopersici; Septoria glycines; Septoria nodorum; Septoria tritici); Setosphaeria (Setosphaeria turcica); Stagonospora(Stagonospora nodorum); Verticillium (Verticillium dahliae; Verticillium albo-atrum).Compositions provided herein comprising Methylobacterium that inhibit plant pathogenic fungi are also expected to be useful in inhibiting fungal growth and/or infection by Fusarium graminearum, Fusarium verticillioides, Fusarium virguliforme, and/or Fusarium proliferatum. Compositions provided herein comprising Methylobacterium that inhibit fungal growth and/or infection by Fusarium graminearum, Fusarium verticillioides, Fusarium virguliforme, and/or Fusarium proliferatum can be used to control infections of cereal plants infected by these fungi. Infections of cereal plants selected from the group consisting of a rice, wheat, corn, barley, millet, sorghum, oat, and rye plants by Fusarium sp can be controlled by the compositions provided herein. In any of the aforementioned embodiments, the plant pathogenic fungus that is inhibited can be in its anamorphic form, its telcomorphic form, or in both its anamorphic and teleomorphic forms. Certain Methylobacterium isolates or combinations of isolates can also be used to inhibit certain plant pathogenic fungi in certain crops as disclosed in Table 3. In certain embodiments where a combination of isolates are used (e.g., NLS0109, a derivative thereof, or a Methylobacterium sp. related thereto and at least one additional Methylobacterium strain selected from the group consisting of NLS0017, NLS0020, NLS0021, NLS0042, NLS0064, NLS0066, NLS0089, a derivative thereof, and a Methylobacterium sp. related thereto), the isolates can be applied either simultaneously or sequentially. In certain embodiments where a combination of isolates are used (e.g., or NLS0109, a derivative thereof, or a Methylobacterium sp. related thereto and at least one additional Methylobacterium strain selected from the group consisting of NLS0017, NLS0020, NLS0021, NLS0042, NLS0064, NLS0066, NLS0089, a derivative thereof, and a Methylobacterium sp. related thereto), the isolates can be applied in either the same mode(s) (e.g., via a seed treatment, a foliar application, or in furrow) or by distinct modes.

TABLE 3
Methylobacterium isolates and combinations of isolates for use in compositions and methods
for controlling certain plant pathogenic fungi in certain crops (including derivatives of the
Methylobacterium isolates or Methylobacterium spp. related thereto)

| NLS Isolate(s) | Crop | Pathogen | Disease Common Name(s) | Mode(s) of Application |
|---|---|---|---|---|
| NLS0109 | Wheat | Fusarium | Fusarium head blight; | Seed treatment; |
| | | graminearum | seedling blight | foliar; or both |
| | | Pythium spp | Pythium root rot | |
| | | Septoria tritici; | Septoria/Stagonospora | |
| | | Stagonospora nodorum | blotch | |
| | | Rhizoctonia solani | Rhizoctonia root rot | |
| | | Fusarium spp. | Fusarium root, crown, | |
| | | | and foot rot | |
| | | Magnaporthe grisea | Head blast | |
| | | Pyrenophora tritici- | Tan Spot | |
| | | repentis | | |
| | | Microdochium nivale | Snow mold | |
| | | Blumeria graminis f. | Powdery mildew | |
| | | sp. tritici | | |
| NLS0109 | Corn | Cercospora zeae- | Gray leaf spot | Seed treatment; |
| | | maydis | | In-furrow; foliar; or |
| | | Colletotrichum | Anthracnose (foliar | both in-furrow and |
| | | graminicola | and stalk rot) | foliar |
| | | Fusarium | Fusarium stalk rot; ear | |
| | | graminearum | rot | |
| | | Fusarium | Fusarium ear rot | |
| | | verticillioides | | |
| | | Rhizoctonia solani | Rhizoctonia crown and | |
| | | | root rot | |
| | | Stenocarpella maydis | Diplodia stalk and ear | |
| | | | rot | |
| | | Pythium spp. | Pythium root rot | |
| | | Sclerospora | Downy mildew; Crazy | |
| | | graminicola; | top | |
| | | Sclerophthora | | |
| | | macrospora | | |
| NLS0109 | Soybean | Cercospora sojina | Frogeye leaf spot | Seed treatment; |
| | | Cercosporia kikuchii | Purple leaf blotch and | in-furrow; foliar; or |
| | | | seed stain | any combination |
| | | Septoria glycines | Brown spot | thereof |
| | | Pythium spp. | Pythium root rot | |
| | | Fusarium spp. | Fusarium seed rot, | |
| | | | blight/wilt, root rot, | |
| | | | and pod and collar rot | |
| | | Sclerotinia | White mold | |
| | | sclerotiorum | | |
| | | Fusarium virguliform | Sudden death | |
| | | | syndrome | |
| | | Rhizoctonia solani | Rhizoctonia damping | |
| | | | off and root rot | |
| | | Peronospora | Downy mildew | |
| | | manshurica | | |
| | | Phytophthora sojae | Root and stem rot | |
| NLS0109 + | Wheat | Fusarium | Fusarium head blight; | Seed treatment; |
| NLS0089 | | graminearum | seedling blight | in-furrow; foliar; or |
| | | Septoria tritici; | Septoria/Stagonospora | any combination |
| | | Stagonospora nodorum | blotch | thereof where |
| | | Pythium spp. | Pythium root rot | isolates are used |
| | | Rhizoctonia solani | Rhizoctonia root rot | alone or in |
| | | Fusarium spp. | Fusarium root, crown, | combination for |
| | | | and foot rot | each treatment. |
| | | Blumeria graminis f. | Powdery mildew | |
| | | sp. tritici | | |
| | | Magnaporthe grisea | Head blast | |
| | | Pyrenophora tritici- | Tan Spot | |
| | | repentis | | |
| | | Microdochium nivale | Snow mold | |
| NLS0109 + | Soybean | Sclerotinia | White mold | Seed treatment; |
| NLS0089 | | sclerotiorum | | in-furrow; foliar; or |
| | | Cercospora sojina | Frogeye leaf spot | any combination |
| | | Cercospora kikuchii | Purple leaf blotch and | thereof, where |
| | | | seed stain | isolates are used |
| | | Fusarium spp. | Fusarium seed rot, | alone or in |
| | | | blight/wilt, root rot, | combination for |
| | | | and pod and collar rot | any treatment. |
| | | Septoria glycines | Brown spot | |
| | | Pythium spp. | Pythium root rot | |
| | | Peronospora | Downy mildew | |
| | | manshurica | | |
| | | Rhizoctonia solani | Rhizoctonia damping | |
| | | | off and root rot | |
| | | Phytophthora sojae | Root and stem rot | |
| | | Fusarium virguliform | Sudden death | |
| | | | syndrome | |
| NLS0109 + | Corn | Pythium spp. | Pythium root and stalk | In-furrow; foliar; or |
| NLS0089 | | | rot | any combination |
| | | Stenocarpella maydis | Diplodia stalk and ear | thereof, where |
| | | | rot | isolates are used |
| | | Sclerospora | Downy mildew; Crazy | alone or in |
| | | graminicola; | top | combination for |
| | | Sclerophthora | | any treatment. |
| | | macrospora | | |
| | | Rhizoctonia solani | Rhizoctonia crown and | |
| | | | root rot | |
| | | Fusarium spp. | Fusarium root and | |
| | | | stalk rot | |
| | | Colletotrichum | Anthracnose leaf blight | |
| | | graminicola | and stalk rot | |
| | | Cercospora zeae- | Gray leaf spot | |
| | | maydis | | |
| NLS0020 + | Wheat | Fusarium | Fusarium head blight; | Seed treatment; |
| NLS0109 | | graminearum | seedling blight | in-furrow; foliar; or |
| | | Septoria tritici; | Septoria/Stagonospora | any combination |
| | | Stagonospora nodorum | blotch | thereof, where |
| | | Pythium spp. | Pythium root rot | isolates are used |
| | | Rhizoctonia solani | Rhizoctonia root rot | alone or in |
| | | Fusarium spp. | Fusarium root, crown, | combination for |
| | | | and foot rot | any treatment, |
| | | Blumeria graminis f. | Powdery mildew | including NLS0020 |
| | | sp. tritici | | applied in-furrow |
| | | Magnaporthe grisea | Head blast | and NLS0109 |
| | | Pyrenophora tritici- | Tan spot | applied foliar. |
| | | repentis | | |
| | | Microdochium nivale | Snow mold | |
| NLS0020 + | Soybean | Sclerotinia | White mold | Seed treatment; |
| NLS0109 | | sclerotiorum | | in-furrow; foliar; or |
| | | Cercospora sojina | Frogeye leaf spot | any combination |
| | | Cercospora kikuchii | Purple leaf blotch and | thereof, where |
| | | | seed stain | isolates are used |
| | | Septoria glycines | Brown spot | alone or in |
| | | Pythium spp. | Pythium root rot | combination for |
| | | Fusarium spp. | Fusarium seed rot, | any treatment, |
| | | | blight/wilt, root rot, | including NLS0020 |
| | | | and pod and collar rot | applied in-furrow |
| | | Peronospora | Downy mildew | and NLS0109 |
| | | manshurica | | applied foliar. |
| | | Rhizoctonia solani | Rhizoctonia damping | |
| | | | off and root rot | |
| | | Phytophthora sojae | Root and stem rot | |
| | | Fusarium virguliform | Sudden death | |
| | | | syndrome | |
| NLS0020 + | Corn | Cercospora zeae- | Gray leaf spot | Seed treatment; |
| NLS0109 | | maydis | | in-furrow; foliar; or |
| | | Colletotrichum | Anthracnose (foliar | any combination |
| | | graminicola | and stalk rot) | thereof, where |
| | | Fusarium | Fusarium stalk rot; ear | isolates are used |
| | | graminearum | rot | alone or in |
| | | Fusarium | Fusarium ear rot | combination for |
| | | verticillioides | | any treatment, |
| | | Rhizoctonia solani | Rhizoctonia crown and | including NLS0020 |
| | | | root rot | applied in-furrow |
| | | Stenocarpella maydis | Diplodia stalk and ear | and NLS0109 |
| | | | rot | applied foliar. |
| | | Pythium spp. | Pythium root rot | |
| | | Sclerospora | Downy mildew; Crazy | |
| | | graminicola; | top | |
| | | Sclerophthora | | |
| | | macrospora | | |
| NLS0017 + | Corn | Pythium spp. | Pythium root and stalk | Seed treatment; |
| NLS0109 | | | rot | in-furrow; foliar; or |
| | | Rhizoctonia solani | Rhizoctonia crown and | any combination |
| | | | root rot | thereof, where |
| | | Stenocarpella maydis | Diplodia stalk and ear | isolates are used |
| | | | rot | alone or in |
| | | Sclerospora | Downy mildew; Crazy | combination for |
| | | graminicola; | top | any treatment, |
| | | Sclerophthora | | including NLS0017 |
| | | macrospora | | applied in-furrow |
| | | Fusarium spp. | Fusarium root and | and NLS0109 |
| | | | stalk rot | applied foliar. |
| | | Colletotrichum | Anthracnose leaf blight | |
| | | graminicola | and stalk rot | |
| | | Cercospora zeae- | Gray leaf spot | |
| | | maydis | | |
| NLS0017 + | Soybean | Sclerotinia | White mold | Seed treatment; |
| NLS0109 | | sclerotiorum | | in-furrow; foliar; or |
| | | Cercospora sojina | Frogeye leaf spot | any combination |
| | | Cercospora kikuchii | Purple leaf blotch and | thereof, where |
| | | | seed stain | isolates are used |
| | | Septoria glycines | Brown spot | alone or in |
| | | Pythium spp. | Pythium root rot | combination for |
| | | Fusarium spp. | Fusarium seed rot, | any treatment. |
| | | | blight/wilt, root rot, | |
| | | | and pod and collar rot | |
| | | Peronospora | Downy mildew | |
| | | manshurica | | |
| | | Rhizoctonia solani | Rhizoctonia damping | |
| | | | off and root rot | |
| | | Phytophthora sojae | Root and stem rot | |
| | | Fusarium virguliform | Sudden death | |
| | | | syndrome | |
| NLS0017 + | Wheat | Fusarium | Fusarium head blight; | Seed treatment; |
| NLS0109 | | graminearum | seedling blight | foliar; or both |
| | | Pythium spp | Pythium root rot | |
| | | Septoria tritici; | Septoria/Stagonospora | |
| | | Stagonospora nodorum | blotch | |
| | | Rhizoctonia solani | Rhizoctonia root rot | |
| | | Fusarium spp. | Fusarium root, crown, | |
| | | | and foot rot | |
| | | Magnaporthe grisea | Head blast | |
| | | Pyrenophora tritici- | Tan Spot | |
| | | repentis | | |
| | | Microdochium nivale | Snow mold | |
| | | Blumeria graminis f. | Powdery mildew | |
| | | sp. tritici | | |
| NLS0064 + | Soybean | Sclerotinia | White mold | Seed treatment; |
| NLS0109 | | sclerotiorum | | in-furrow; foliar; or |
| | | Cercospora sojina | Frogeye leaf spot | any combination |
| | | Cercospora kikuchii | Purple leaf blotch and | thereof, where |
| | | | seed stain | isolates are used |
| | | Septoria glycines | Brown spot | alone or in |
| | | Pythium spp. | Pythium root rot | combination for |
| | | Fusarium spp. | Fusarium seed rot, | any treatment. |
| | | | blight/wilt, root rot, | |
| | | | and pod and collar rot | |
| | | Peronospora | Downy mildew | |
| | | manshurica | | |
| | | Rhizoctonia solani | Rhizoctonia damping | |
| | | | off and root rot | |
| | | Phytophthora sojae | Root and stem rot | |
| | | Fusarium virguliform | Sudden death | |
| | | | syndrome | |
| NLS0021 + | Corn | Pythium spp. | Pythium root and stalk | Seed treatment; |
| NLS0109 | | | rot | in-furrow; foliar; or |
| | | Rhizoctonia solani | Rhizoctonia crown and | any combination |
| | | | root rot | thereof, where |
| | | Stenocarpella maydis | Diplodia stalk and ear | isolates are used |
| | | | rot | alone or in |
| | | Sclerospora | Downy mildew; Crazy | combination for |
| | | graminicola; | top | any treatment, |
| | | Sclerophthora | | including NLS0021 |
| | | macrospora | | applied in-furrow |
| | | Fusarium spp. | Fusarium root and | and NLS0109 |
| | | | stalk rot | applied foliar. |
| | | Colletotrichum | Anthracnose leaf blight | |
| | | graminicola | and stalk rot | |
| | | Cercospora zeae- | Gray leaf spot | |
| | | maydis | | |

In certain embodiments, an amount of a composition provided herein that is sufficient to provide for inhibition of fungal infection in a plant or plant part can be a composition with Methylobacterium that inhibit plant pathogenic fungi at a titer of at least about 5×10⁸ colony-forming units per milliliter, at least about 1×10⁹ colony-forming units per milliliter, at least about 1×10¹⁰ colony-forming units per milliliter, or at least about 3×10¹⁰ colony-forming units per milliliter. In certain embodiments, an amount of a composition provided herein that is sufficient to provide for inhibition of fungal disease in a plant or plant part can be a composition with Methylobacterium that inhibit plant pathogenic fungi at a titer of about 5×10⁸ colony-forming units per milliliter to at least about 6×10¹⁰ colony-forming units per milliliter. In certain embodiments, an amount of a composition provided herein that is sufficient to provide for inhibition of fungal disease in a plant or plant part can be a fermentation broth product with a Methylobacterium that inhibit plant pathogenic fungi titer of a solid phase of that product is at least about 1×10⁷, 5×10⁷, 1×10⁸, or 5×10⁸ colony-forming units per gram to at least about 6×10¹⁰, 1×10¹³, or 5×10¹³ colony-forming units of Methylobacterium per gram of the solid phase wherein a mono-culture or co-culture of Methylobacterium that inhibit plant pathogenic fungi is adhered thereto. In certain embodiments, an amount of a composition provided herein that is sufficient to provide for inhibition of fungal disease in a plant or plant part can be a composition with a Methylobacterium titer of at least about 1×10⁷, 5×10⁷, 1×10⁸, or 5×10⁸ colony-forming units per gram to at least about 6×10¹⁰, 1×10¹³, or 5×10¹³ colony-forming units of Methylobacterium per gram of particles in the composition containing the particles that comprise a solid substance wherein a mono-culture or co-culture of Methylobacterium that inhibit plant pathogenic fungi is adhered thereto. In any of the aforementioned compositions, the indicated concentrations can be fungal inhibitory concentrations.

In certain embodiments, the compositions, plants and plant parts treated with the compositions, and methods provided herein can comprise fungicides in addition to the Methylobacterium strains for enhanced disease control. In certain embodiments, the fungicides are provided in compositions comprising the Methylobacterium, for example as seed treatments, as slurries for in furrow treatment or in foliar sprays. In certain embodiments, the fungicides are provided as separate compositions for use in the methods provided herein. Examples of fungicides that can be used in the compositions and methods provided herein for enhanced control of plant pathogenic fungi are provided in Table 4.

TABLE 4

| | | | Chemicals to Combine with |
|---|---|---|---|
| | | | Methylobacterium for |
| Crop | Pathogen | Disease Common Name(s) | Enhanced Disease Control |
| Wheat | Fusarium | Fusarium head blight; | tebuconazole, triticonazole, |
| | graminearum | seedling blight | metconazole, ipconazole, |
| | | | prothioconazole |
| Wheat | Pythium spp | Pythium root rot | metalaxyl, mefenoxam, |
| | | | oxathiapiprolin, ethaboxam, |
| | | | thiram, thiophanate-methyl |
| Wheat | Blumeria graminis f. | Powdery mildew | propiconazole, triadimenfon, |
| | sp. tritici | | fluxapyroxad, (trifloxystrobin, |
| | | | fluopyram specialty crops) |
| Wheat | Septoria tritici; | Septoria/Stagonospora | tebuconazole, triticonazole, |
| | Stagonospora | blotch | metconazole, ipconazole, |
| | nodorum | | prothioconazole |
| Wheat | Rhizoctonia | Rhizoctonia root rot | tebuconazole, triticonazole, |
| | solani | | metconazole, ipconazole, |
| | | | prothioconazole, thiram, captan, |
| | | | penflufen, carboxin, fluxapyroxad |
| Wheat | Fusarium spp. | Fusarium root, crown, and | tebuconazole, triticonazole, |
| | | foot rot | metconazole, ipconazole, |
| | | | prothioconazole, thiram, captan, |
| | | | penflufen, imazalil, carboxin, |
| | | | thiabendazole, fluxapyroxad |
| Wheat | Magnaporthe | Head blast | prothioconazole, trifloxystrobin |
| | grisea | | |
| Wheat | Pyrenophora | Tan Spot | tebuconazole, triticonazole, |
| | tritici-repentis | | metconazole, ipconazole, |
| | | | prothioconazole |
| Wheat | Microdochium | Snow mold | dimethomorph, boscalid |
| | nivale | | |
| Corn | Cercospora | Gray leaf spot | azoxystrobin, pyraclostrobin, |
| | zeae-maydis | | fluoxastrobin, and trifloxystrobin, |
| | | | fenamidone |
| Corn | Colletotrichum | Anthracnose (foliar and | ipconazole, prothioconazole, |
| | graminicola | stalk rot) | propiconazole, azoxystrobin, |
| | | | pyraclostrobin, fluoxastrobin, and |
| | | | trifloxystrobin |
| Corn | Fusarium | Fusarium stalk rot; ear rot | tebuconazole, triticonazole, |
| | graminearum | | metconazole, ipconazole, |
| | | | prothioconazole, thiram, captan, |
| | | | penflufen, imazalil, carboxin, |
| | | | thiabendazole, fluxapyroxad |
| Corn | Fusarium | Fusarium ear rot | tebuconazole, triticonazole, |
| | verticillioides | | metconazole, ipconazole, |
| | | | prothioconazole, thiram, captan, |
| | | | penflufen, imazalil, carboxin, |
| | | | thiabendazole, fluxapyroxad |
| Corn | Fusarium spp. | Fusarium root and stalk rot | tebuconazole, triticonazole, |
| | | | metconazole, ipconazole, |
| | | | prothioconazole, thiram, captan, |
| | | | penflufen, imazalil, carboxin, |
| | | | thiabendazole, fluxapyroxad |
| Corn | Stenocarpella | Diplodia stalk and ear rot | tebuconazole, triticonazole, |
| | maydis | | metconazole, ipconazole, |
| | | | prothioconazole, thiram, captan, |
| | | | penflufen, carboxin, fluxapyroxad |
| Corn | Pythium spp. | Pythium root and stalk rot | metalaxyl, mefenoxam, |
| | | | oxathiapiprolin, ethaboxam, |
| | | | thiram, thiophanate-methyl |
| Corn | Sclerospora | Downy mildew; Crazy top | propamocarb, dimethomorph, |
| | graminicola; | | metalaxyl, mefenoxam |
| | Sclerophthora | | |
| | macrospora | | |
| Corn | Rhizoctonia | Rhizoctonia crown and | tebuconazole, triticonazole, |
| | solani | root rot | metconazole, ipconazole, |
| | | | prothioconazole, thiram, captan, |
| | | | penflufen, carboxin, fluxapyroxad |
| Soybean | Cercospora | Frogeye leaf spot | azoxystrobin, pyraclostrobin, |
| | sojina | | fluoxastrobin, and trifloxystrobin |
| Soybean | Cercosporia | Purple leaf blotch and seed | azoxystrobin, pyraclostrobin, |
| | kikuchii | stain | fluoxastrobin, and trifloxystrobin, |
| | | | fenamidone |
| Soybean | Septoria | Brown spot | azoxystrobin, pyraclostrobin, |
| | glycines | | fluoxastrobin, and trifloxystrobin, |
| | | | fluopyram |
| Soybean | Pythium spp. | Pythium root rot | metalaxyl, mefenoxam, |
| | | | oxathiapiprolin, ethaboxam |
| Soybean | Sclerotinia | White mold | tebuconazole, prothioconazole, |
| | sclerotiorum | | fluopyram |
| Soybean | Fusarium spp. | Fusarium seed rot, | tebuconazole, triticonazole, |
| | | blight/wilt, root rot, and | metconazole, ipconazole, |
| | | pod and collar rot | prothioconazole, thiram, captan, |
| | | | penflufen, imazalil, carboxin, |
| | | | thiabendazole, fluxapyroxad |
| Soybean | Fusarium | Sudden death syndrome | fluopyram |
| | virguliform | | |
| Soybean | Rhizoctonia | Rhizoctonia damping off | tebuconazole, triticonazole, |
| | solani | and root rot | metconazole, ipconazole, |
| | | | prothioconazole, thiram, captan, |
| | | | penflufen, carboxin, fluxapyroxad |
| Soybean | Phytophthora | Root and stem rot | metalaxyl, mefenoxam, |
| | sojae | | oxathiapiprolin |

When chemicals are used in combination with Methylobacterium, application rates of the chemicals may be reduced compared to standard commercial application rates for a given plant species, while providing the same or increased level of protection to the plant from plant pathogenic fungal pathogens. In certain embodiments, Methylobacterium strains provided here provide enhanced activity in combination with fungicides as the result of activity against particular fungal strains for which the chemical fungicides provide no or incomplete control. In certain embodiments, the Methylobacterium is NLS0109 or NL0089, the fungus is Cercospora, the crop is corn, the disease is grey leaf spot, and the chemical is a strobilurin, such as pyraclostrobin, or an azole, such as tebuconazole. In certain embodiments, the fungicide application rate or numbers of applications of the fungicide can be reduced as the result of antifungal activity of the applied Methylobacterium. In certain embodiments, the Methylobacterium is NLS0109, the fungus is Pythium, the crop is soybean or corn, the disease is Pythium root rot, and the chemical is metalaxyl, mefenoxam, oxathiapiprolin or ethaboxam. In certain embodiments, the fungicide application rate or numbers of applications of the fungicide can be reduced as the result of antifungal activity of the applied Methylobacterium.

### EXAMPLES

The following examples are included to demonstrate various embodiments. It will be appreciated by those of skill in the art that the techniques disclosed in the following examples represent techniques discovered by the Applicants to function well. However, those of skill in the art should, in light of the instant disclosure, appreciate that many changes can be made in the specific embodiments that are disclosed, while still obtaining like or similar results, without departing from the scope of the disclosure.

### Example 1. Corn and Soybean Field Trials Summer of 2015

In the summer of 2015, field trials to evaluate disease suppression in corn and soybeans by PPFMs were performed at two independent locations: Bethel, Missouri and Troy, Ohio. Both trial locations were managed by contract research organizations. NewLeaf Symbiotics personnel visited each site at least twice to ensure proper trial implementation. The same strains and application rates were tested at both locations. The trials were arranged as a split-plot within an RCBD (randomized complete block design) with six replications at the Bethel site and four replications at the Troy site. Treatments for corn are described in Table 5 and treatments for soybean are described in Table 6. In-furrow treatments were applied at a rate of 1,250 mL 10X PPFM concentrate per acre and foliar treatments were applied at a rate of 5,000 mL 10X PPFM concentrate per acre. The split-plot design allowed for the evaluation of in-furrow treatment, foliar treatment, response to sequential PPFM treatments, and interactions between different PPFMs. Data from the Troy, Ohio soybean trial were not analyzed.

TABLE 5
2015 Pathology Corn Field Trial Treatments

| Treatment Number | Whole-plot treatment | Sub-plot treatment |
|---|---|---|
| 1 | Mock | Mock |
| 2 | NLS0020 | Mock |
| 3 | Mock | NLS0020 |
| 4 | NLS0020 | NLS0020 |
| 5 | Mock | NLS0109 |
| 6 | NLS0020 | NLS0109 |
| 7 | Mock | NLS0066 |
| 8 | NLS0020 | NLS0066 |

TABLE 6
2015 Pathology Soybean Field Trial Treatments

| Treatment Number | Whole-plot treatment | Sub-plot treatment |
|---|---|---|
| 1 | Mock | Mock |
| 2 | NLS0089 | Mock |
| 3 | Mock | NLS0020 |
| 4 | NLS0089 | NLS0020 |
| 5 | Mock | NLS0109 |
| 6 | NLS0089 | NLS0109 |
| 7 | Mock | NLS0066 |
| 8 | NLS0089 | NLS0066 |

At each site, conventional row spacing was used and standard agronomic practices were followed. Corn and soy hybrids with similar genetics but suitable for the specific trial locations were supplied for each site. Sub-plot sizes were no less than four 20′ rows. A five-foot border was left between sub-plot to mitigate neighbor effects. Additionally, observations were taken from only the center two rows of each plot. Whole-plots consisted of the four sub-plots plus five foot borders between plots. Trial locations were selected in areas with natural disease pressure and no artificial inoculations were made. As a result, the same diseases were not evaluated at each location. Diseases rated in corn were anthracnose (Colletotrichum graminicola), grey leaf spot (Cercospora zeae-maydis), and common rust (Puccinia sorghi). Diseases rated in soybean were brown spot (Septoria glycines) and various foliar diseases, particularly frogeye leafspot (Cercospora sojina). For each disease present, incidence and/or severity ratings were collected and analyzed to determine treatment effects.

Disease ratings and statistical analysis results are reported in Tables 7-9. Due to the different disease ratings and replication number at each site, data from the two trial locations were analyzed separately. Data analyses were performed using SAS JMP software v11.2 (SAS Institute, Cary, NC). Data were analyzed according to JMP guidelines for split-plot analysis within the ‘Fit Model’ function, which uses the REML technique for mixed models. Student's T and Tukey's HSD post hoc tests were applied to determine differences between treatment groups (α=0.05). Contrasts were used to make comparisons between specific groups of interest.

TABLE 7
Soybean Foliar Disease-Bethel, Missouri

| | | Brown | Brown | | |
|---|---|---|---|---|---|
| | | spot | spot | Leaf spot | Leaf spot |
| Whole-plot | Sub-plot | severity | severity | severity | severity |
| (in-furrow) | (foliar) | early | late | early | late |
| Treatment | Treatment | (%) | (%) | (%) | (%) |
| Mock | Mock | 3.00 | 8.33 | 1.67 | 5.50 |
| Mock | NLS0020 | 1.33^{T} | 4.17^{T,H} | 0.17^{T,H} | 2.00^{T,H} |
| Mock | NLS0066 | 1.67^{T} | 4.83^{T} | 0.67^{T} | 4.17 |
| Mock | NLS0109 | 1.67^{T} | 5.67^{T} | 0.50^{T,H} | 4.00^{T} |
| NLS0089 | Mock | 1.17^{T} | 5.00^{T} | 0.17^{T,H} | 2.50^{T,H} |
| NLS0089 | NLS0020 | 1.17^{T} | 5.17^{T} | 0.33^{T,H} | 2.50^{T,H} |
| NLS0089 | NLS0066 | 1.67 | 5.17^{T} | 0.33^{T,H} | 4.33 |
| NLS0089 | NLS0109 | 2.00 | 5.00^{T} | 0.33^{T,H} | 3.00^{T} |

^{T}Treatment significantly different from control (Mock, Mock) by Student's T-test (α = 0.05)
^{H}Treatment significantly different from control (Mock, Mock) by Tukey's HSD (α = 0.05)

TABLE 8
Corn Foliar Disease-Bethel, Missouri

| | | Anthrac- | Gray leaf | Gray leaf | Common |
|---|---|---|---|---|---|
| Whole-plot | Sub-plot | nose | spot | spot | rust |
| (in-furrow) | (foliar) | severity | severity | severity | severity |
| Treatment | Treatment | (%) | early (%) | late (%) | (%) |
| Mock | Mock | 21.17 | 3.17 | 13.00 | 12.17 |
| Mock | NLS0020 | 18.83 | 2.00^{T,H} | 11.33 | 11.67 |
| Mock | NLS0066 | 9.50^{T,H} | 1.83^{T,H} | 10.83^{T} | 10.50 |
| Mock | NLS0109 | 9.67^{T,H} | 2.50^{T} | 11.00^{T} | 10.00^{T} |
| NLS0020 | Mock | 18.67 | 2.17^{T} | 11.83 | 10.67 |
| NLS0020 | NLS0020 | 17.83 | 2.00^{T} | 11.50 | 11.33 |
| NLS0020 | NLS0066 | 9.17^{T} | 1.00^{T,H} | 9.50^{T,H} | 9.67^{T} |
| NLS0020 | NLS0109 | 9.17^{T} | 1.17^{T,H} | 9.83^{T,H} | 9.17^{T,H} |

^{T}Treatment significantly different from control by Student's T-test (α = 0.05)
^{H}Treatment significantly different from control by Tukey's HSD (α = 0.05)

TABLE 9
Corn Foliar Disease-Troy, Ohio

| | | Gray leaf | Tip | Tip | Stalk |
|---|---|---|---|---|---|
| Whole-plot | Sub-plot | spot | dieback | dieback | rot |
| (in-furrow) | (foliar) | severity | severity | incidence | severity |
| Treatment | Treatment | (%) | (%) | (%) | (%) |
| Mock | Mock | 67.50 | 11.00ⁱ | 0.17ⁱ | 1.55 |
| Mock | NLS0020 | 67.50 | 8.25 | 0.14 | 1.60 |
| Mock | NLS0066 | 62.50 | 12.50 | 0.18 | 1.45 |
| Mock | NLS0109 | 60.00 | 11.75 | 0.19 | 1.40 |
| NLS0020 | Mock | 65.00 | 7.50* | 0.12* | 1.85 |
| NLS0020 | NLS0020 | 67.50 | 10.00 | 0.16 | 1.55 |
| NLS0020 | NLS0066 | 60.00 | 9.00 | 0.14 | 1.45 |
| NLS0020 | NLS0109 | 62.50 | 9.25 | 0.14 | 1.85 |

^{I}The average across all mock in-furrow treatments was significantly different from the average across all NLS0020 in-furrow treatments by contrast (α = 0.05)
*Treatment significantly different from control (Mock, Mock) by contrast (α = 0.10)

All treatments applied to soybeans demonstrated disease suppression against both brown spot (Septoria glycines) and other foliar leaf spot diseases. Foliar application of NLS0020 without in-furrow treatment resulted in the lowest rating for all diseases and was the most effective treatment for suppression of disease relative to the control. Foliar application of NLS0020 following NLS0089 in-furrow treatment also demonstrated disease suppression across all treatments. Foliar treatment of NLS0109 significantly reduced all diseases and provided greater suppression when applied after in-furrow treatment with NLS0089 for all applications except the carly brown spot rating. In-furrow treatment with NLS0089 alone significantly reduced all diseases and had a particularly strong effect against foliar leaf spot diseases.

In corn at the Bethel, Missouri site, foliar applications of NLS0066 and NLS0109 significantly suppressed all diseases, with the exception of NLS0066 against common rust. In-furrow application of NLS0020 improved the disease suppression provided by NLS0066 and NLS0109 foliar applications in all examples. This demonstrates enhanced efficacy through multiple applications of these specific PPFM strains. Application of NLS0020 alone, including foliar, in-furrow, and in-furrow followed by foliar, resulted in reduction of carly Gray leaf spot severity, but had no significant impact on the other diseases tested.

At the Troy, Ohio location, the NLS0066 and NLS0109 foliar applications provided the lowest gray leaf spot severity ratings. This was in agreement with the treatment effects observed in Missouri. In-furrow application of NLS0020 alone suppressed both the severity and incidence of tip dieback, which can be indicative of an effect on disease and abiotic stressors. Additionally, all applications of in-furrow NLS0020 combined suppressed tip dieback metrics relative to all mock in-furrow treatments combined, indicating an overall positive effect of NLS0020 in-furrow treatment.

### Example 2. Suppression of White Mold by Methylobacterium

Sclerotinia sclerotiorum is a polyphagous ascomycete fungus, with a host range that encompasses thousands of dicot plants. White mold on soybean and other leguminous crops is of particular agronomic importance. Under cool, moist environmental conditions, white mold causes premature senescence and drastically reduced yields. There is no available complete genetic resistance to white mold and partial resistance is only marginally effective. Further, fungicide applications specifically for white mold are only applied in years when disease is highly likely and must be applied within a narrow window in order to provide effective protection. Application of beneficial bacteria that colonize the plant and provide enhanced disease resistance could mitigate effects of white mold and increase the efficacy of genetic and chemical disease management tactics.

Frozen PPFM stock solutions at a 10X concentration of approximately 1×10⁸ CFU/mL were thawed to room temperature directly prior to foliar treatment. PPFM cells were washed by centrifuging to pellet the cells, pouring off the resulting supernatant, re-suspending the cells in one mL sterile distilled water, centrifuging and pouring off the supernatant a second time, then re-suspending a second time in 1 mL of sterile distilled water. Re-suspended, washed cells were then diluted to a 1X concentration. Within two hours of PPFM preparation, an airbrush calibrated to 20 psi was used to apply 10mL of 1X PPFM solution evenly across each flat of treatment plants. PPFMs were applied to three-weck old plants and inoculations were performed the following week when plants were approximately one-month old.

Seeds were planted into either potting media, field soil, or a 50/50 mix of potting media and field soil, depending on the specific experiment. In all experiments, flats holding 18 pots each were used and the pots containing individual treatments were organized into randomized complete blocks either at planting or just prior to inoculation. Immediately after planting, pots were moved to a greenhouse (75-80° F.; RH 40-90%; 16h day-length) and grown there for one month. Plants were watered daily and received supplemental fertilizer two times per weck.

One-month old plants were inoculated with 5-7 day-old cultures of Sclerotinia sclerotiorum grown on PDA in the dark. A modified version of the cut petiole inoculation technique was used (Hoffman et al. 2002. Plant Dis. 86:971-980). Briefly, the petiole of the third trifoliate was cut with scissors approximately one inch from the stem. The broad end of a 1000 μL pipet tip was used to excise an agar disk from the outer edge of an S. sclerotiorum culture. The tip was then placed over the cut petiole such that the broad end of the pipet tip was in contact with the stem and petiole base and the cut end of the petiole was in contact with the mycelium side of the agar plug. A small piece of parafilm was wrapped around the tip and stem to prevent the tip from falling off. Inoculated plants were incubated in the greenhouse for 7-10 days to allow for disease development prior to rating.

Lesion length and wilt severity were collected as disease metrics. Length of brown or bleached lesions was measured using a ruler. Wilt severity was rated on a 0-5 scale with 0 indicating a completely health plant and 5 indicating a dead plant.

A total of 12 different PPFM strains in five individual experiments, cach replicated three times, were tested for suppression of white mold following foliar PPFM application (Table 10). Of these strains, only NLS0020 and NLS0109, both of which were in the first two experiments, had a significant effect on either lesion length or severity of wilt caused by pathogen infection (Table 11). In the first experiment, which had a lower sample size, no significant effects were detected but both NLS0020 and NLS0109 decreased white mold lesion length and wilt severity by at least 20% relative to the mock-treated control. In the second experiment, NLS0020 decreased lesion length by approximately 30% (P=0.04) but did not have a significant effect on wilt severity (10% reduction; P=0.76). NLS0109 decreased lesion length by approximately 40% (P<0.01) and wilt by approximately 40% (P=0.06). For analysis, a Dunnett's test was conducted in the ‘Fit Y by X’ function of SAS JMP v12.0 statistical analysis software. The mock-foliar treatment application group, which received a bacterial growth medium only foliar application, was designated as the control group.

TABLE 10
Lesion Length (LL) and Wilt Severity of White Mold
Following Foliar Application of PPFM Strains

| | | | | |
|---|---|---|---|---|
| Strain | LL ± SE | LL RTC (%) | Wilt ± SE | Wilt RTC (%) |

Experiment 1

| | | | | |
|---|---|---|---|---|
| Control | 100.42 ± 9.55 | 100.00 | 4.46 ± 0.26 | 100.00 |
| NLS0020 | 74.50 ± 10.65 | 74.19 | 3.33 ± 0.39 | 74.67 |
| NLS0071 | 84.79 ± 12.66 | 84.44 | 3.54 ± 0.41 | 79.37 |
| NLS0109 | 79.17 ± 09.90 | 78.84 | 3.58 ± 0.41 | 80.26 |

Experiment 2

| | | | | |
|---|---|---|---|---|
| Control | 106.04 ± 11.18 | 100.00 | 4.13 ± 0.34 | 100.00 |
| NLS0020 | 74.17 ± 8.67 | 69.94 | 3.71 ± 0.35 | 89.90 |
| NLS0071 | 94.38 ± 10.77 | 89.00 | 4.12 ± 0.32 | 101.01 |
| NLS0109 | 64.38 ± 10.81 | 60.71 | 2.92 ± 0.45 | 70.71 |

Experiment 3

| | | | | |
|---|---|---|---|---|
| Control | 45.00 ± 6.12 | 100.00 | 2.19 ± 0.33 | 100.00 |
| NLS0017 | 55.33 ± 7.12 | 123.00 | 2.83 ± 0.32 | 129.00 |
| NLS0037 | 50.63 ± 7.02 | 112.50 | 2.19 ± 0.38 | 100.00 |
| NLS0089 | 45.16 ± 6.52 | 100.35 | 1.94 ± 0.33 | 88.57 |

Experiment 4

| | | | | |
|---|---|---|---|---|
| Control | 63.75 ± 8.16 | 100.00 | 3.22 ± 0.33 | 100.00 |
| NLS0046 | 54.22 ± 9.33 | 85.05 | 2.34 ± 0.33 | 72.82 |
| NLS0064 | 67.81 ± 9.07 | 106.37 | 2.88 ± 0.34 | 89.32 |
| NLS0066 | 59.38 ± 9.03 | 93.13 | 2.56 ± 0.35 | 79.61 |

Experiment 5

| | | | | |
|---|---|---|---|---|
| Control | 79.38 ± 7.52 | 100.00 | 3.72 ± 0.30 | 100.00 |
| IP0021 | 97.26 ± 7.35 | 122.53 | 4.39 ± 0.21 | 117.97 |
| IP0121 | 95.78 ± 6.77 | 120.67 | 4.16 ± 0.17 | 111.76 |
| IP0237 | 84.38 ± 6.58 | 106.30 | 4.16 ± 0.24 | 111.76 |

TABLE 11
Analysis of White Mold Control by PPFM Strains

| | LL | | Wilt | |
|---|---|---|---|---|
| Strain | (% control) | LL p-value | (% control) | Wilt p-value |
| NLS0071 | 11.00 | 0.6975 | −1.01 | 0.9996 |
| NLS0020 | 30.06 | 0.0429 | 10.10 | 0.7625 |
| NLS0109 | 39.29 | 0.0053 | 29.29 | 0.0589 |

Foliar applications of PPFM strain NLS0109 reduced symptoms of both lesion length and wilt on soybean caused by the white mold pathogen Sclerotinia sclerotiorum. Applications of this strain could be used as a standalone treatment for suppression of white mold. Further, NLS0109 could be mixed with chemical or biological disease management tools as part of an integrated disease management approach. Such combinations could be leveraged to increase and/or prolong product efficacy and as part of a resistance management program.

### Example 3. Suppression of Gray Leaf Spot by Methylobacterium spp. in Combination and Comparison with Conventional Fungicides

Gray leaf spot, caused by the ascomycete fungal pathogen Cercospora zeae-maydis, is a serious disease of field corn. While varying levels of resistance to this disease are present in commercial germplasm, no complete resistance is available. Gray leaf spot is a perennial problem and is particularly severe during summers with high relative humidity. The summer of 2016 was favorable for gray leaf spot, with disease pressure starting in June and persisting throughout the growing season.

In this trial, a greenhouse study was conducted to determine the efficacy of PPFM strains when applied alone, in combination with each other, or in combination with low label rates of the foliar fungicide Headline™ (active ingredient—pyraclostrobin; BASF Corporation, Research Triangle, NC, USA). These treatments were compared against Headline application alone, as well as other agriculturally relevant fungicide treatment and treatment combinations containing pyraclostrobin as an active ingredient (Table 12).

TABLE 12
PPFM/Industry Standard Greenhouse Trial Treatments

| No. | Treatment | Rate | | Timing |
|---|---|---|---|---|
| 1 | Untreated control | 0 | fl oz/A | N/A |
| 2 | Headline ™-Foliar | 6 | fl oz/A | VT |
| 3 | Fortix ™ ¹-Foliar | 4 | fl oz/A | VT |
| 4 | Xanthion ™ ²-In-furrow | 0.6 fl oz/A Component A | | Planting |
| | | 3.0 fl oz/A Component B | | |
| 5 | Xanthion ™ In-furrow | 0.6 fl oz/A Component A | | Planting |
| | | 3.0 fl oz/A Component B | | |
| | Headline ™-Foliar | 6 | fl oz/A | VT |
| 6 | NLS0020-In-furrow | 1,250 | mL/A | Planting |
| 7 | NLS0020-In-furrow | 1,250 | mL/A | Planting |
| | Headline ™-Foliar | 6 | fl oz/A | VT |
| 8 | NLS0109-Foliar | 5,000 | mL/A | VT |
| 9 | NLS0109-Foliar | 5,000 | mL/A | VT |
| | Headline ™-Foliar | 6 | fl oz/A | VT |
| 9 | NLS0020-In-furrow | 1,250 | mL/A | Planting |
| | NLS0109-Foliar | 5,000 | mL/A | VT |
| 10 | NLS0020-In-furrow | 1,250 | mL/A | Planting |
| | NLS0109-Foliar | 5,000 | mL/A | VT |
| | Headline ™-Foliar | 6 | fl oz/A | VT |

¹ Fortix ™ (Arysta Life Sciences, Cary, North Carolina, USA)
² Xanthion ™ (BASF Corporation, Research Triangle, NC, USA)

The trial was conducted in Spring-Summer 2016 with corn planted in early April and inoculated two-weeks prior to anthesis. Inoculum was applied by placing pathogen-infected corn debris around the base of each plant and then increasing relative humidity within the greenhouse to 60-90% during the day and greater than 90% at night, with the temperature ranging from 70-82° F. To promote infection, a gray leaf spot susceptible corn hybrid (Sun Prairie 617RR) was used for the trial. Disease ratings were taken at 14, 21, and 28 days post-inoculation and a final ear weight was collected for each plant.

The trial was arranged as an RCBD with 10 replications. In-furrow treatments were applied at planting using a hand-held sprayer and application volumes were scaled down to the total pot area. Fungicides were mixed and applied in accordance with label instructions. PPFMs were supplied as frozen concentrate that was thawed just prior to application. Extra seeds were planted and treated for each treatment group to ensure a total of ten replicates were available for inoculation. Two weeks after germination, corn seedlings were transferred from seedlings trays to 2.5 gallon pots, in which they grown for the remainder of the experiment. Foliar applications were performed at the VT growth-stage using a backpack sprayer. To prevent drift within the greenhouse, plants in cach treatment group were brought outside and the treatment was applied to the entire group. Plants were then returned to the greenhouse and randomized into the ten experimental replicates.

Disease data were analyzed in JMP version 12.0 (SAS Institute, Cary, NC). Disease incidence and severity values collected at each sampling data were used to calculate disease index [(incidence*severity)/100] and a cumulative area under the disease progress curve (AUDPC) was calculated the three disease index values for each treatment. Data were analyzed using the ‘Fit Model’ function in JMP with ‘Rep’ as a random effect and ‘Treatment’ as a fixed effect. A post-hoc Tukey test at an alpha level of 0.05 was applied to perform means separation between treatments (Table 13).

PPFM treatments alone did not perform differently from the UTC or from in-furrow treatment with Xanthion™. Xanthion™ is a combination product of pyraclostrobin and the Bacillus subtilis microbial inoculant strain MBI600; thus, PPFM strains alone performed similarly to a currently available commercial product that combines a chemical fungicide with a microbial agent. The combination of NL0020-IF/NL0109-F reduced disease relative to the UTC and disease levels under this treatment were not significantly different from those of either foliar-applied pyraclostrobin-based fungicide (Headline™ and Fortix™). NL0020-IF/Headline-F™ suppressed disease to levels similar to those of Xanthion-IF™/Headline-F™, Headline-F™, and Fortix-F™. As the Xanthion™/Headline™ combination requires two applications of pyraclostrobin, similar performance by NLS0020/Headline indicates that an application of this active ingredient could be dropped in favor of a PPFM application without any adverse effects on disease severity.

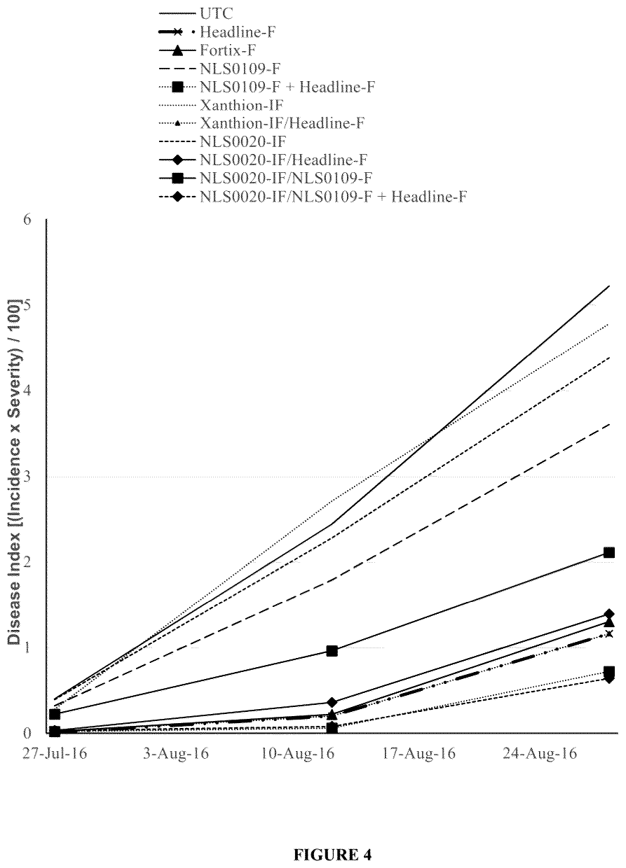

Of all treatments, NLS0109-F/Headline-F™ and NLS0020-IF/NLS0109-F+Headline-F™ had the greatest effects on disease suppression relative the UTC. In particular, these treatments numerically reduced disease to levels greater than Headline™ alone at the 21-and 14-day rating intervals (FIG. 4). This indicates the potential for PPFM applications, particularly NLS0109 foliar applications made in combination with Headline™, to extend the window of protection provided by conventional fungicides used for gray leaf spot mitigation.

In addition to disease suppression, PPFM application had a strong positive effect on ear weight (Table 12). The average weight for all treatments with PPFMs was 132.67 grams/ear, which is nearly ten grams higher than the 123.00 grams/ear average of the untreated control and fungicide-only treatments. Fortix™ alone was the only fungicide treatment to perform similarly to PPFM treatments and NLS0020 in-furrow application was particularly conducive to increased yield weight, garnering three of the four highest weights obtained.

This experiment demonstrates the potential for use of PPFMs in combination with conventional fungicides. PPFMs can be used to increase product efficacy by prolonging the window of effective protection offered by currently available chemistries and by offering an alternate product for use in resistance management. Additionally, PPFMs had a positive effect on ear weight, indicating a potential to boost yields when used alone or in combination with conventional fungicides.

TABLE 13
Gray Leaf Spot Greenhouse Evaluation Results

| | | % Dif- | | % Dif- | |
|---|---|---|---|---|---|
| | Yield | ference | | ference | LSMeans |
| | (grams/ | from | | from | Comparison |
| Treatment | ear) | UTC | AUDPC | UTC | (Tukey) |
| UTC | 120 | 0.00 | 39.55 | 0.00 | A |
| Headline-F ™ | 121 | 0.83 | 5.57 | −85.92 | BC |
| Fortix-F ™ | 135 | 12.50 | 6.3 | −84.07 | BC |
| NLS0109-F | 127 | 5.83 | 28.49 | −27.96 | A |
| NLS0109-F + | 129 | 7.50 | 3.15 | −92.04 | C |
| Headline-F ™ | | | | | |
| Xanthion-IF ™ | 116 | −3.33 | 38.64 | −2.30 | A |
| Xanthion-IF ™/ | 123 | 2.50 | 5.67 | −85.66 | BC |
| Headline-F ™ | | | | | |
| NLS0020-IF | 135 | 12.50 | 35.39 | −10.52 | A |
| NLS0020-IF/ | 138 | 15.00 | 7.72 | −80.48 | BC |
| Headline-F ™ | | | | | |
| NLS0020-IF/ | 131 | 9.17 | 16.44 | −58.43 | B |
| NLS0109-F | | | | | |
| NLS0020-IF/ | 136 | 13.33 | 3.12 | −92.11 | C |
| NLS0109-F + | | | | | |
| Headline-F ™ | | | | | |

### Example 4. Grey Leaf Spot Field Trials Summer 2016

A second year of corn pathology field trials was conducted in 2016. Trials were placed in sites with naturally high levels of gray leaf spot inoculum and a susceptible corn hybrid (Sun Prairie 617RR) was used to facilitate gray leaf spot infection. Two trial locations were included: Dana, Iowa and Bethel, Missouri. At each site, the trial was conducted as a randomized complete block with six replicates. Standard local agronomic practices for fertilizer application, tillage, row spacing, population, and pest management were used. Standard weed and insect management practices were employed and seed was supplied to cooperators pre-treated with a standard fungicide/insecticide seed treatment package (Acceleron™ 2016 Corn, Monsanto, St. Louis, MO, USA). Foliar fungicide applications were only made if specified in the trial protocol. Sixteen treatments were included in the trials, including PPFMs applied alone, PPFMs applied in combination, PPFMs applied alongside chemical fungicides, and fungicide alone standards (Table 14). PPFM compositions were diluted to a concentration of approximately 109 CFU/ml and applied at the rates shown in Table 14. Assessments collected included early season stand and vigor, mid-season gray leaf spot incidence and severity, flowering date, stalk diameter, lodging, final stand at harvest, yield, and moisture content. Incidence and severity data from each rating date were combined to provide a disease index value [(incidence*severity)/100]. Data from each site were analyzed separately used JMP v12.0 (SAS Institute, Cary, NC). Treatment effects on response variables of interest were determined using the ‘Fit Model’ function with ‘Treatment’ specified as a fixed effect and ‘Rep’ as a random effect. Post-hoc means separation were performed using the ‘LSMeans Differences Student's T’ option within ‘Fit Model’ and means are reported as LSMeans from the corresponding model. At the Bethel, Missouri site the Untreated check was the lowest yielding treatment and maintained the highest levels of gray leaf spot disease incidence and severity throughout the growing season (Table 15). Treatments with NLS0109 provided a greater than 10 bu/A yield increase over the Untreated check. Disease index values for both treatments with NLS0109 were significantly lower (P<0.05) than the Untreated check on the first two rating dates (August 12 and August 21) but on the final rating date (August 28) only the treatment combination of NLS0020 in-furrow followed by an NLS0109 foliar treatment suppressed disease relative to the Untreated check (Table 15).

TABLE 14
2016 Gray Leaf Spot Field Trial Treatments

| Trt | | | | |
|---|---|---|---|---|
| No | Treatment(s) | Application Timing | Rate | |
| 1 | Untreated Control | N/A | N/A | |
| 2 | Headline ™ | VT¹ | 6.0 | fl oz/A |
| 3 | Fortix ™ | VT | 4/0 | fl oz/A |
| 4 | Xanthion ™ | In-furrow @ planting | 0.6 fl oz/A Component A | |
| | | | 3.0 fl oz/A Component B | |
| | Headline ™ | VT | 6 | fl oz/A |
| 5 | NLS0020 | In-furrow @ planting | 1.25 | L/A |
| 6 | NLS0020 | In-furrow @ planting | 1.25 | L/A |
| | NLS0066 | VT | 5.0 | L/A |
| 7 | NLS0020 | In-furrow @ planting | 1.25 | L/A |
| | NLS0089 | VT | 5.0 | L/A |
| 8 | NLS0020 | In-furrow @ planting | 1.25 | L/A |
| | NLS0109 | VT | 5.0 | L/A |
| 9 | NLS0020 | In-furrow @ planting | 1.25 | L/A |
| | Headline ™ | VT | 6 | fl oz/A |
| 10 | NLS0020 | In-furrow @ planting | 1.25 | L/A |
| | Fortix ™ | VT | 4.0 | fl oz/A |
| 11 | NLS0021 | In-furrow @ planting | 1.25 | L/A |
| 12 | NLS0021 | In-furrow @ planting | 1.25 | L/A |
| | NLS0066 | VT | 5.0 | L/A |
| 13 | NLS0021 | In-furrow @ planting | 1.25 | L/A |
| | NLS0089 | VT | 5.0 | L/A |
| 14 | NLS0021 | In-furrow @ planting | 1.25 | L/A |
| | NLS0109 | VT | 5.0 | L/A |
| 15 | NLS0021 | In-furrow @ planting | 1.25 | L/A |
| | Headline ™ | VT | 6.0 | fl oz/A |
| 16 | NLS0021 | In-furrow @ planting | 1.25 | L/A |
| | Fortix | VT | 4.0 | fl oz/A |

¹VT stage of development: last branch of tassel completely visible but silks not visible.

TABLE 15
Summer 2016 Gray Leaf Spot Field Trial Results-Bethel, Missouri

| Treatment | Yield | | Index_12 Aug. | | Index_21 Aug. | | Index_28 Aug. | |
|---|---|---|---|---|---|---|---|---|
| Untreated Check | 141.77¹ | C² | 7.25 | A | 20.83 | A | 24.83 | A |
| NLS0020 | 149.93 | BC | 4.85 | B | 15.27 | B | 21.33 | AB |
| NLS0020/NLS0066 | 161.20 | AB | 2.83 | C | 12.28 | BCD | 20.07 | BC |
| NLS0020/NLS0089 | 159.88 | AB | 2.08 | CD | 12.37 | BCD | 18.53 | BC |
| NLS0020/NLS0109 | 155.15 | ABC | 2.65 | CD | 10.97 | CD | 19.07 | BC |
| NLS0020/Fortix ™ | 160.32 | AB | 0.38 | F | 1.78 | E | 8.55 | D |
| NLS0020/Headline ™ | 161.62 | AB | 0.83 | EF | 2.17 | E | 5.88 | D |
| NLS0021 | 157.32 | AB | 4.00 | B | 13.15 | BC | 19.88 | BC |
| NLS0021/NLS0066 | 159.47 | AB | 1.57 | DE | 8.87 | D | 16.3 | C |
| NLS0021/NLS0089 | 152.57 | BC | 2.18 | CD | 11.3 | BCD | 20.58 | ABC |
| NLS0021/NLS0109 | 153.40 | ABC | 2.45 | CD | 11.62 | BCD | 20.88 | AB |
| NLS0021/Fortix ™ | 164.22 | AB | 0.25 | F | 1.03 | E | 6.28 | D |
| NLS0021/Headline ™ | 167.23 | A | 0.23 | F | 1.57 | E | 4.83 | D |
| Fortix ™ | 161.62 | AB | 0.4 | F | 1.5 | E | 7.23 | D |
| Headline ™ | 163.78 | AB | 0.42 | F | 1.2 | E | 5.57 | D |
| Xanthion ™ | 167.27 | A | 0.17 | F | 0.9 | E | 5.52 | D |

¹Values reported are means from six replicated plots per treatment
²Means followed by the same capital letter are not significantly different from one another by LSMeans Differences Student's T post-hoc means comparison test (alpha = 0.05)

### Example 5 Detection of Methylobacterium Isolates on Target Crops

Assays are disclosed for detection of specific Methylobacterium strains and closely related derivatives on target crops.

A qPCR Locked Nucleic Acid (LNA) based assay for NLS109 is developed as follows. NLS109 genomic DNA sequence is compared by BLAST analysis of approximately 300bp fragments using a sliding window of from 1-25 nucleotides to whole genome sequences of over 1000 public and proprietary Methylobacterium isolates. Genomic DNA fragments were identified that had weak BLAST alignments, indicative of approximately 60-95% identity over the entire fragment, to corresponding fragments from NLS0109. Target fragments from the NLS0109 genome corresponding to the identified weak alignments regions that were selected for assay development are provided as SEQ ID NOS: 9-11

TABLE 16
Target Fragment Sequences of NLS0109

| | SEQ | |
|---|---|---|
| | ID | |
| Fragment | NO | Sequence |
| ref1_135566 | 9 | ACGGTCACCCCACGGACTGGGCGAGTACCTCACCGGTGTTC |
| | | TATCATAACGCCGAGTTAGTTTTCGACCGTCCCTTATGCGA |
| | | TGTACCACCGGTGTCGGCAGCCGATTTCGTCCCACCGGGAG |
| | | CTGGCGTTCCGGTTCAGACCACCATCATCGGTCACGATGTC |
| | | TGGATTGGACACGGGGCCTTCATCTCCCCCGGCGTGACTAT |
| | | AGGAAACGGCGCGATCGTCGGGGCCCAGGCGGTCGTCACA |
| | | AGAGATGTCCCACCCTATGCGGTAGTTGCTGGCGTCCCCGC |
| | | GACCGTACGACGAT |
| ref1_135772 | 10 | CCAATAAAAGCGTTGGCCGCCTGGGCAACCCGATCCGAGC |
| | | CTAAGACTCAAAGCGCAAGCGAACACTTGGTAGAGACAGC |
| | | CCGCCGACTACGGCGTTCCAGCACTCTCCGGCTTTGATCGG |
| | | ATAGGCATTGGTCAAGGTGCCGGTGGTGATGACCTCGCCC |
| | | GCCGCAAGCGGCGAATTACTCGGATCAGCGGCCAGCACCT |
| | | CGACCAAGTGTCGGAGCGCGACCAAAGGGCCACGTTCGAG |
| | | GACGTTTGAGGCGCGACCAGTCTCGATAGTCTCATCGTCGC |
| | | GGCGAAGCTGCACCTCGA |
| ref1_169470 | 11 | CGATGGCACCGACCTGCCATGCCTCTGCCGTCCGCGCCAGA |
| | | ATGGTAAAGAGGACGAAGGGGGTAAGGATCGTCGCTGCAG |
| | | TGTTGAGCAGCGACCAGAGAAGGGGGCCGAACATCGGCAT |
| | | CAAACCTCGATTGCCACTCGGACGCGAAGCGCGTCTTGAA |
| | | GGAGGGATGGAAGCGAAACGGCCGCAGAGTAACCGCCGA |
| | | CGAAAGATTGCACCCCTCATCGAGCAGGATCGGAGGTGAA |
| | | GGCAAGCGTGGGTTATTGGTAAGTGCAAAAAATATAATGG |
| | | TAGCGTCAGATCTAGCGTTC |

Regions in SEQ ID NOS: 9-11 where corresponding regions in other Methylobacterium strains were identified as having one or more nucleotide mismatches from the NLS109 sequence were selected, and qPCR primers designed using Primer3 software (Untergasser et al., 2012, Koressaar et al., 2007) to flank the mismatch regions, have a melting temperature (Tm) in the range of 53-58 degrees, and to generate a PCR DNA fragment of approximately 100 bp. The probe sequence was designed with a 5′ FAM reporter dye, a 3′ Iowa Black FQ quencher, and contains one to six LNA bases (Integrated DNA Technologies, Coralville, Iowa). At least 1 of the LNA bases is in the position of a mismatch, while the other LNA bases are used to raise the Tm. The Tm of the probe sequence is targeted to be 10 degrees above the Tm of the primers.

Primer and probe sequences for detection of specific detection of NLS0109 are provided as SEQ ID NOS: 12-20 in Table 17. Each of the probes contains a 5′ FAM reporter dye and a 3′ Iowa Black FQ quencher.

TABLE 17
Primer and Probe Sequences for Specific Detection of NLS0109

| | SEQ | |
|---|---|---|
| | ID | |
| Primer/Probe | NO | Sequence* |
| NLS0109_ref1_135566_forward | 12 | CCTCACCGGTGTTCTATCATAAC |
| NLS0109_ref1_135566_reverse | 13 | CCGATGATGGTGGTCTGAAC |
| NLS0109_ref1_135566_probe | 14 | CGTCCCTTATGCGATGTACCA |
| NLS0109_ref1_135772_forward | 15 | GATCCGAGCCTAAGACTCAAAG |
| NLS0109_ref1_135772_reverse | 16 | GACCAATGCCTATCCGATCAA |
| NLS0109_ref1_135772_probe | 17 | AACACTTGGTAGAGACAGCC |
| NLS0109_ref1_169470_forward | 18 | AAGGAGGGATGGAAGCGAAAC |
| NLS0109_ref1_169470_reverse | 19 | ATAACCCACGCTTGCCTTC |
| NLS0109_ref1_169470_probe | 20 | CGCAGAGTAACCGCCGACGAA |

*Bold and underlined letters represent the position of an LNA base

Use of Primer/Probe Sets on Isolated DNA to Detect NLS0109 and Distinguish from Related Methylobacterium Isolates

A qPCR reaction is conducted in 20 μl and contains 10 μl of 2× KiCqStartυ Probe qPCR ReadyMix™, Low ROXTM from Sigma (Cat#KCQS05-1250RXN), 1 μl of 20× primer-probe mix (final concentration of primers is 0.5 μM each and final concentration of probe is 0.25 μM), and 9 μl of DNA template/water. Approximately 30-40 ng of DNA template is used per reaction. The reaction is conducted in a Stratagene Mx3005P qPCR machine with the following program: 950C for 3 min, then 40 cycles of 950C for 15 sec and 600C for 1 min. The

MxPro software on the machine calculates a threshold and Ct value for each sample. Each sample was run in triplicate on the same qPCR plate. A positive result is indicated where the delta Ct between positive and negative controls is at least 5.

Use of the three primer/probe sets to distinguish NLS0109 from closely related isolates by analysis of isolated DNA is shown in Table 18 below. The similarity score shown for the related isolates takes into account both the average nucleotide identity and the alignment fraction between the isolates and NLS0109. One of the tested strains, NLS0730, was used as an additional positive control. It was isolated from a culture of NLS0109, was confirmed by full genome sequencing as identical to NLS0109, and scored positive in all three reactions. The similarity score of greater than 1.000 for this strain is likely the result of a slightly different assembly of the genome for this isolate compared to NLS0109. The delta Ct of approximately 15 or more between the NLS0109 and NLS0730 isolates and the water only control is consistent with the sequence confirmation of the identity of these isolates. Analysis of other isolates that are less closely related to NLS0109 results in delta Ct values similar to those for the water only control.

TABLE 18

| | Similarity | | | |
|---|---|---|---|---|
| | score to | Average Ct Value | | |
| NLS# | NLS0109 | Ref1_135566 | Ref1_135772 | Ref1_169470 |
| NLS0730 | 1.005 | 21.08 | 21.31 | 20.35 |
| NLS0109 | 1 | 21.97 | 22.62 | 22.08 |
| NLS0731 | 0.181 | No Ct | 37.85 | >37.91 |
| NLS0644 | 0.87 | >36.8 | >38.31 | No Ct |
| NLS0700 | 0.88 | >38.36 | >38.36 | >38.44 |
| NLS0710 | 0.894 | No Ct | >37.47 | >38.13 |
| NLS0834 | 0.852 | 37.81 | No Ct | 37.97 |
| NLS0939 | 0.862 | 37.94 | 38.37 | >38.35 |
| NLS0947 | 0.807 | 38.44 | No Ct | No Ct |
| NLS1015 | 0.894 | 38.77 | No Ct | >37.91 |
| NLS1217 | 0.872 | 37.64 | 37.20 | 37.96 |
| H2O only | | >38.14 | >35.92 | >37.12 |

### Use of Primer/Probes for Detection of NLS109 on Treated Plant Materials

### Detection of NLS0109 on Seed Washes From Treated Soybean Seeds.

NLS0109 can be detected and distinguished from other Methylobacterium isolates on treated soybean seeds as follows. Soybean seeds were treated with Methylobacterium isolates from 10x frozen glycerol stock to obtain a final concentration of 10⁶ CFU/seed. Becker Underwood Flo Rite 1706 polymer is used to improve adhesion. An uninoculated control containing polymer and water is used. DNA is isolated from the seeds as follows. Approximately 25 ml of treated seeds are submerged for 5 minutes in 20 ml 0.9% sterile saline. Tubes are vortexed for 15 minutes, then the seed wash is removed to a new tube. An additional 10 ml 0.9% sterile saline is added to the same seeds, vortexed briefly, and combined with the previous seed wash. The seed wash liquid is centrifuged. The loose pellet is saved and transferred to smaller tubes, while the supernatant is discarded. The sample is centrifuged again, and the final sample obtained as an approximately 100 μl loose pellet. The 100 μl pellet is used as the input for DNA extraction using MOBio UltraClean Microbial DNA Extraction kit Cat#12224-250. As shown in Table 19, NLS0109 and NLS0730, are detected in seed washes from treated soybean seeds using all 3 primer probe sets, as demonstrated by delta Ct of greater than 10 as compared to Ct values of negative controls.

TABLE 19

| | Similarity | | | |
|---|---|---|---|---|
| | score to | Average Ct Value | | |
| Treatment | NLS0109 | Ref1_135566 | Ref1_135772 | Ref1_169470 |
| NLS0109 | 1 | 18.07 | 17.49 | 17.95 |
| control | N/A | 34.80 | 33.72 | 33.59 |
| (polymer | | | | |
| only) | | | | |
| NLS0730 | 1.005 | 17.76 | 17.03 | 17.54 |
| NLS0731 | 0.181 | 33.67 | 32.70 | 32.43 |

Detection of NLS0109 on Leaves from Plants Grown from Treated Soybean Seeds.

Soybean seeds were treated with Methylobacterium isolates NLS0109, NS0730, and NLS0731 from 10× frozen glycerol stock to obtain a final concentration of 10⁶ CFU/seed. Becker Underwood Flo Rite 1706 polymer is used to improve adhesion. An uninoculated control contained polymer and water. Seeds were planted in field soil mix, placed in a growth chamber for approximately two weeks, and watered with unfertilized RO water every 1-2 days to keep soil moist. After 2 weeks of growth, true leaves from about 9 plants were harvested into sterile tubes. Each treatment had at least 2 reps in each experiment, and each experiment was grown at least 3 times.

DNA from bacteria on the harvested leaves is isolated as follows. Leaves are submerged for 5 minutes in buffer containing 20 mM Tris, 10 mM EDTA, and 0.024% Triton X-100. Tubes are vortexed for 10 minutes, and then sonicated in two 5 minute treatments (10 minutes total). Leaf tissue is removed, and the remaining liquid centrifuged. The loose pellet is saved and transferred to smaller tubes, while the supernatant is discarded. The sample is centrifuged again, and the final sample obtained as an approximately 100 μl loose pellet. The 100 μl pellet is used as the input for DNA extraction using MOBio UltraClean Microbial DNA Extraction kit Cat#12224-250. The average yield of DNA is 50-60 ng/μl in 30 μl. As shown in Table 20, NLS0109 and NLS0730, are detected on leaves harvested from plants grown from soybean seeds treated with the Methylobacterium strains using all 3 primer probe sets, as demonstrated by delta Ct values of around 5.

TABLE 20
Average of 3 experiments each with 3 biological replicates

| | Similarity | | | |
|---|---|---|---|---|
| | score to | Average Ct Value | | |
| Treatment | NLS0109 | Ref1_135566 | Ref1_135772 | Ref1_169470 |
| NLS0109 | 1.000 | 35.00 | 34.67 | 34.00 |
| control | N/A | 39.67 | 39.67 | 39.33 |
| (polymer | | | | |
| only) | | | | |
| NLS0730 | 1.005 | 35.00 | 35.00 | 34.00 |
| NLS0731 | 0.181 | 40.00 | 39.67 | 40.00 |

For detection of NLS0109 foliar spray treatment on corn: Untreated corn seeds were planted in field soil in the growth chamber, and watered with non-fertilized R.O. water. After plants germinated and grew for approximately 3 weeks, they were transferred to the greenhouse. At V5 stage, plants were divided into 3 groups for treatment: foliar spray of NLS0109, mock foliar spray, and untreated. Plants receiving the foliar spray of NLS0109 were treated with 10x glycerol stock at the rate of 71.4 μl per plant using Solo sprayers. This converts to the rate of 10L/acre in the field. Mock treated plants were sprayed with 71.4 μl water/plant. Untreated plants received no foliar spray treatment. Leaves were harvested two weeks after foliar spray treatment into sterile tubes and DNA from bacteria on the harvested leaves is isolated as described above. Each experiment was grown at least 2 times. As shown in Table 21, NLS0109 is detected on leaves harvested from corn plants treated by a foliar spray application of the Methylobacterium strains using all 3 primer probe sets, as demonstrated by delta Ct values of approximately 10 between the sample and the negative controls.

TABLE 21

| | Average Ct Value | | |
|---|---|---|---|
| Treatment | Ref1_135566 | Ref1_135772 | Ref1_169470 |
| Control (no | 32.43 | 32.10 | 31.55 |
| application) | | | |
| Control (mock | 35.54 | 35.34 | 34.80 |
| application) | | | |
| NLS0109 | 23.36 | 22.88 | 22.66 |
| (10 L/acre | | | |
| equivalent) | | | |

The above results demonstrate the use of genome specific primers and probes to detect Methylobacterium strain NLS0109 on various plant tissues following treatment with the strains, and provide methods to distinguish NLS0109 from closely related isolates. Similar methods are developed for additional Methylobacterium strains, NLS0020, NLS0017 and NLS0089 using target sequence fragments and primer/probe pairs as shown in Tables 22-27 below.

TABLE 22
Target Fragment Sequences of NLS0020

| | SEQ | |
|---|---|---|
| | ID | |
| Fragment | NO | Sequence |
| ref3_25009 | 21 | GCCCTTCTGTCAGGCGATATTGTATAATGGCGTTGCCCCAA |
| | | TAGAAGCAGCCATTCGTGCGAGGGCAGCAGCGACGCTAGG |
| | | TCGAAAGAGCATCCTAATCTCGATCAAGATGCGACTGAGA |
| | | TTTCTGATGAAAATATCTAGACACAAGCAAAGCTGGTGAA |
| | | ATTACAACGATCATGGCGACAATTGCGGCCAATTCGGCCG |
| | | GAACTTGAAGGAACATAAAAATGAATATTACAAATATACC |
| | | GCAAAGCATGTAGAGTTGCTACACCAAGGGTCGGGACGTC |
| | | CAAAAAAACTCACTGAGGA |
| ref3_25219 | 22 | GGAACATAAAAATGAATATTACAAATATACCGCAAAGCAT |
| | | GTAGAGTTGCTACACCAAGGGTCGGGACGTCCAAAAAAAC |
| | | TCACTGAGGAAGTCGACTGGAAGCACGAGGCGCCCCCCCC |
| | | AGGAGCGGGGCGACCGGCAAGGGGGCCCGCAATTGTCGCC |
| | | ATGATCGACCAGCTTAGGTAGGATCCTCTTTCGACCTAACG |
| | | AATGGCTGCTTCTATTGGGGCAACGCCATTATACAATATCG |
| | | CCTGACCATCTGGAACGCGGCCCGGTCCACCGGCAGGTTG |
| | | GCGACGACAGCGTCGGAG |
| ref1_4361220 | 23 | CGGCGTCGACCAGCCGGGCGAACTGCTTGGGCATGCTCTCC |
| | | CGCGACGCCGGCCACAGCCGCGTCCCCGTCCCTCCGCACA |
| | | GGATCATCGGGTGGATTTGAAAGGCAAAACGGGACATCAG |
| | | GATAGGCCGCTCAGGCGTTGGCGCTGAGGCGCTTGATGTC |
| | | GGCGTCGACCATCTCGGTGATCAGCGCCTCGAGGCTGGTCT |
| | | CGGCCTCCCAGCCGAAGGTCGCCTTGGCCTTGGCGGGGTTG |
| | | CCCAGCAGCACCTCGACCTCTGCCGGCCGGAACAGCGCCG |
| | | GGTCGACGATCAGGTGG |
| ref1_4602420 | 24 | CTGGACATGCGCCCACCCCGGCCAAGTCCGACCGCACCGG |
| | | CAACCGCTCCTGTAGTCGTCGTCATCGTTCTCACCCCTGAG |
| | | GCGGAGACCGTCCGCTAACGGGGTGTCTCAAGCAACCGTG |
| | | GGGCGGAGGAACACGCACGTAGTCGCGTTTCAAGGTTCGC |
| | | ACGAACGCCTCGGCCATGCCGTTGCTCTGCGGGCTCTCCAG |
| | | CGGCGTCGTTTTTGGCACCAAACCAAGGTCGCGGGCGAAG |
| | | CGGCGCGTGTCGCGGGGACTGTCAGGAATTTCGTGTGGGG |
| | | GCGGCCATAGTGGATCCG |

TABLE 23
Primer and Probe Sequences for
Specific Detection of NLS0020

| | SEQ | |
|---|---|---|
| | ID | |
| Primer/Probe | NO | Sequence* |
| NLS0020_ref3_25009_5′ | 25 | CACAAGCAAAGCTGGTGA |
| NLS0020_ref3_25009_3′ | 26 | AAGATATGCTTTGCGGGTA |
| NLS0020_ref3_25009_probe | 27 | CGATCATGGCGACAATTG |
| NLS0020_ref3_25219_5′ | 28 | CAATATCGCCTGACAGAAGG |
| NLS0020_ref3_25219_3′ | 29 | CACTTCAACAAAGGCGATCA |
| NLS0020_ref3_25219_probe | 30 | TTGGCGACGACAGC |
| NLS0020_ref1_4361220_5′ | 31 | ACTGCTTGGGCATGCTCTC |
| NLS0020_ref1_4361220_3′ | 32 | CCTATCCTGATGTCCCGTTT |
| NLS0020_ref1_4361220_probe | 33 | AGGATCATCGGGTGGATTTG |
| NLS0020_ref1_4602420_5′ | 34 | AGGAACACGCACGTAGTC |
| NLS0020_ref1_4602420_3′ | 35 | CCACACGAAATTCCTGAC |
| NLS0020_ref1_4602420_probe | 36 | TGGCACCAAACCAA |

*Bold and underlined letters represent the position of an LNA base

TABLE 24
Target Fragment Sequences of NLS0017

| | SEQ | |
|---|---|---|
| Fragment | ID NO | Sequence |
| ref4_930 | 37 | GCAAAACGACCTAATAGTTCTACAGCGGCATGCGCCAAGT |
| | | CAGCGCGGTGAACAGTATACCTGGGAGCAACTTGTCCTCC |
| | | GAAACCCACATAAAACAAATTACTCCTGGCAGTGCCCAGT |
| | | CCATCAAAATCGAATACAATATTTCTCGAGGAGGCATCTGT |
| | | AATAGCCTGCCAAAGCAACAAAGCTATGGCGCCGTTATGA |
| | | CTTTCATTGCTTCTGGTAGACATAAAATAATATGCCGATTT |
| | | GTGATCCCAAATGTAGAATATTGCCGCATCAATTGCGCCAA |
| | | GTTTATTTCGGATCGAT |
| ref1_142021 | 38 | GGCGCCAACGGTATGATCGCATGATTTTCCTGCGGCATAGC |
| | | TTGCGGGAATGGCGTATTTGGCGCTCTCCTCAGGAATTTCT |
| | | AAGGGCATACGCAGGAACTCTACAGCACTTTTACTGGTATT |
| | | TTGTAGTGACAGCGGAGGAGGCTGGTGCTCAAGGTAATCG |
| | | TGATGAAGTGATCCGGGCCATTCGGGGCGCGTTTCTAGTCT |
| | | TTCCAATCCGCGCCCTGTACCACGTATTACGCCGGACCGGT |
| | | CTGCGCCGCGCCGCCCTCTTGACCGCCCTAAATGTCTAAGA |
| | | GCGTCTAACAAAGC |
| ref1_142636 | 39 | GACGATATCGCTCATCTTCACTGCATTGAAGCTGGTGCCGT |
| | | ACTGCATAGGGATGAAAAAGTGATGCGGATAGACGGCTGA |
| | | CGGGAAAGCGCCTGGTCGATCGAAGACTTTGCTGACGAGG |
| | | TTGTGGTAGCCCCGGATATAGGCATCGAAGGCCGGGACGT |
| | | TGATCCCATCCTTTGCCTTATCTTGACTGGCGTCGTCGCGTG |
| | | CCGTCAGAACGGGCACGTCGCAGGTCATCGAGGCCAGCAC |
| | | CTTGCGGAACACCTGCGTTCCGCCGTTGGGATTATCGACGG |
| | | CGAACGCGGTGGCCGC |

TABLE 25
Primer and Probe Sequences for Specific Detection of NLS0017

| | SEQ | |
|---|---|---|
| Primer/Probe | ID NO | Sequence* |
| NLS0017_ref4_930_forward | 40 | GTCCTCCGAAACCCACATAAA |
| NLS0017_ref4_930_reverse | 41 | CTACCAGAAGCAATGAAAGTCAT |
| NLS0017_ref4_930_probe | 42 | TCTGTAATAGCCTGCCAAAGCA |
| NLS0017_ref1_142021_forward | 43 | GGCTGGTGCTCAAGGTAAT |
| NLS0017_ref1_142021_reverse | 44 | ACATTTAGGGCGGTCAAGAG |
| NLS0017_ref1_142021_probe | 45 | ATGAAGTGATCCGGGCCAT |
| NLS0017_ref1_142636_forward | 46 | CCGTACTGCATAGGGATGAAA |
| NLS0017_ref1_142636_reverse | 47 | TAAGGCAAAGGATGGGATCAA |
| NLS0017_ref1_142636_probe | 48 | TTGCTGACGAGGTTGTGGTAG |

*Bold and underlined letters represent the position of an LNA base

TABLE 26
Target Fragment Sequences of NLS0089

| | SEQ | |
|---|---|---|
| | ID | |
| Fragment | NO | Sequence |
| ref1_194299 | 49 | GGAAATCGGCTTCAAGTACGACGTCACGCCGGCCATGCAG |
| | | GTCACGGGTGCACTGTTCAATCTCGAGCGCGACAACCAGC |
| | | CGTTCCCCTCGAACGTGGAGTCCGGCCTCGTCCTTGGCGCA |
| | | GGTCAGACACGCACCCAGGGCGCGGAAATCGGCCTGGCCG |
| | | GCTATCTAACCGATTGGTGGCAGGTCTTTGGCGGCTACGCT |
| | | TATACCGAGGCACGCGTACTCTCGCCACTGGAAGACGATG |
| | | GAGACGTGATCGCAGCAGGTAATCTCGTCGGCAACGTTCC |
| | | GCTAAATACTTTCAGTCT |
| ref1_194305 | 50 | CGGCCTGGCCGGCTATCTAACCGATTGGTGGCAGGTCTTTG |
| | | GCGGCTACGCTTATACCGAGGCACGCGTACTCTCGCCACTG |
| | | GAAGACGATGGAGACGTGATCGCAGCAGGTAATCTCGTCG |
| | | GCAACGTTCCGCTAAATACTTTCAGTCTGTTCAACAAGTTC |
| | | GATATCAACGAGAATTTCTCCGTTGCTCTGGGCTATTACTA |
| | | TCAGGATGCCAGCTTTGCCTCCTCAGACAATGCAGTGCGTT |
| | | TGCCAAGTTATTCGCGGTTCGATGGCGGGTTGTTCTATCGA |
| | | TTCGACGAGTTGAC |
| ref1_194310 | 51 | ACGTTCCGCTAAATACTTTCAGTCTGTTCAACAAGTTCGAT |
| | | ATCAACGAGAATTTCTCCGTTGCTCTGGGCTATTACTATCA |
| | | GGATGCCAGCTTTGCCTCCTCAGACAATGCAGTGCGTTTGC |
| | | CAAGTTATTCGCGGTTCGATGGCGGGTTGTTCTATCGATTC |
| | | GACGAGTTGACACGCGTTCAGCTTAGCGTCGAGAACATTTT |
| | | CGACAGGCGTTACATCATCAACTCCAACAACAACAACAAC |
| | | CTCACGCCTGGCGCGCCGAGAACAGTCCGCGTGCAATTGA |
| | | TCGCTCGGTTCTAAA |

TABLE 27
Primer and Probe Sequences for Specific Detection of NLS0089

| | SEQ | |
|---|---|---|
| | ID | |
| Primer/Probe | NO | Sequence* |
| NLS0089_ref1_194299_forward | 52 | TTTGGCGGCTACGCTTATAC |
| NLS0089_ref1_194299_reverse | 53 | AACGTTGCCGACGAGATTAC |
| NLS0089_ref1_194299_probe | 54 | AGACGATGGAGACGTGATCGCA |
| NLS0089_ref1_194305_forward | 55 | GGCAACGTTCCGCTAAATAC |
| NLS0089_ref1_194305_reverse | 56 | AAAGCTGGCATCCTGATAGT |
| NLS0089_ref1_194305_probe | 57 | CGAGAATTTCTCCGTTGCTCTG |
| NLS0089_ref1_194310_forward | 58 | CGATGGCGGGTTGTTCTAT |
| NLS0089_ref1_194310_reverse | 59 | AGGCGTGAGGTTGTTGTT |
| NLS0089_ref1_194310_probe | 60 | AGGCGTTACATCATCAACTCCA |

*Bold and underlined letters represent the position of an LNA base

### Example 6 Grey Leaf Spot Field Trials Summer 2017

The effect of Methylobacterium treatment on grey leaf spot disease of corn was evaluated in corn field trials. The trials were conducted as a randomized complete block with six replicates. Standard local agronomic practices for fertilizer application, tillage, row spacing, population, and pest management were used. Standard weed and insect management practices were employed and seed was supplied to cooperators pre-treated with a standard fungicide/insecticide seed treatment package (Acceleron™ 2016 Corn, Monsanto, St. Louis, MO, USA). Foliar fungicide applications were only made if specified in the trial protocol and shown in the tables below. When seed was pretreated with Methylobacterium, it was provided from a composition comprising from 10⁹-10¹⁰ CFU/ml to provided approximately 10⁶ CFU/seed. For in furrow or foliar applications, PPFM compositions were diluted to a concentration of approximately 10⁹ CFU/ml and applied at the rates shown in the tables below.

In one trial, treatments with NLS0109 in combination with NLS0017 or NLS0020 were evaluated. Methylobacterium strains were applied either as seed treatment or in furrow applications. Disease severity and yield results are provided in Table 28 below. Treatment with NLS0109/NLS0017 in furrow provided a 15 bu/acre yield increase over the untreated check. Treatment with NLS0109/NLS0020 as a seed treatment provided a greater than 10 bu/acre yield increase over the untreated check. Each of these treatments resulted in a yield that was approximately the same (or slightly increased) as for corn treated with Fortix (strobilurin plus triazole).

TABLE 28
Reduction of Grey Leaf Spot Disease
with Combinations of PPFM Strains

| | | Yield | |
|---|---|---|---|
| GSF-04 2017 | Application | (bu/A) | GLS Severity |
| UNT | | 221 | 6.3 |
| NLS 17/109 0.625 liter/A | In furrow | 236 | 7.9 |
| NLS 20/109 0.625 liter/A | In furrow | 222 | 7 |
| NLS 17/109 | Seed treatment | 222 | 7.9 |
| NLS 20/109 | Seed treatment | 232 | 7.6 |
| Fortix 4.5 fl oz/A | In furrow | 228 | 5.9 |
| Monsoon 4.5 fl oz/A | Foliar @ VT | 237 | 6.7 |
| Headline 10 fl. Oz/A | Foliar @ VT | 254 | 4.9 |
| Average | | 231 | 6.775 |

In a second trial, NLS0089 and NLS0109 were applied at different application rates alone or in combination with commercial fungicides also applied at various rates. All applications were by foliar spray at the VT stage of development. Disease severity and yield results are provided in Tables 29 and 30 below.

TABLE 29
NLS 89 and 109 Applied in Combination
With Headline (Pyraclostrobin)

| | Application Rate | | bu/A | GLS Severity |
|---|---|---|---|---|
| UNT | | | 241 | 42.3 |
| Headline 0.5 | 4 | oz/A | 253 | 17.7 |
| Headline 1.0 | 8 | oz/A | 257 | 19.4 |
| NLS 89 1.0 | 2.5 | Liters/A | 232 | 32.9 |
| Headline 0.5/NLS 89 1.0 | 4 | oz/A | 248 | 20.2 |
| | 2.5 | Liters/A | | |
| Headline 1.0/NLS 89 1.0 | 8 | oz/A | 249 | 21.1 |
| | 2.5 | Liters/A | | |
| NLS 89 0.5 | 1.25 | Liters/A | 238 | 41.9 |
| Headline 0.5/NLS 89 0.5 | 4 | oz/A | 249 | 21.4 |
| | 1.25 | Liters/A | | |
| Headline 1.0/NLS 89 0.5 | 8 | oz/A | 258 | 18.9 |
| | 1.25 | Liters/A | | |
| NLS 109 1.0 | 2.5 | Liters/A | 241 | 40.6 |
| Headline 0.5/NLS 109 1.0 | 4 | oz/A | 254 | 20.8 |
| | 2.5 | Liters/A | | |
| Headline 1.0/NLS 109 1.0 | 8 | oz/A | 258 | 18.4 |
| | 2.5 | Liters/A | | |
| NLS 109 0.5 | 1.25 | Liters/A | 229 | 43.4 |
| Headline 0.5/NLS 109 0.5 | 4 | oz/A | 254 | 23.8 |
| | 1.25 | Liters/A | | |
| Headline 1.0/NLS 109 0.5 | 8 | oz/A | 243 | 17.1 |
| | 1.25 | Liters/A | | |

TABLE 30
NLS 89 and 109 Applied in Combination
With Monsoon (Tebuconazole)

| | | | bu/A | GLS Severity % |
|---|---|---|---|---|
| UNT | | | 231 | 49.9 |
| Monsoon 0.5 | 2 | oz/A | 231 | 43.0 |
| Monsoon 1.0 | 4 | oz/A | 237 | 43.2 |
| NLS 89 1.0 | 2.5 | Liters/A | 231 | 41.3 |
| Mon 0.5/89 1.0 | 2 | oz/A | 236 | 37.8 |
| | 2.5 | Liters/A | | |
| Mon 1.0/89 1.0 | 4 | oz/A | 239 | 40.8 |
| | 2.5 | Liters/A | | |
| NLS 89 0.5 | 1.25 | Liters/A | 234 | 47.6 |
| Mon 0.5/89 0.5 | 2 | oz/A | 232 | 45.2 |
| | 1.25 | Liters/A | | |
| Mon 1.0/89 0.5 | 4 | oz/A | 234 | 34.2 |
| | 1.25 | Liters/A | | |
| NLS 109 1.0 | 2.5 | Liters/A | 238 | 41.1 |
| Mon 0.5/109 1.0 | 2 | oz/A | 232 | 41.3 |
| | 2.5 | Liters/A | | |
| Mon 1.0/109 1.0 | 4 | oz/A | 233 | 40.5 |
| | 2.5 | Liters/A | | |
| NLS 109 0.5 | 1.25 | Liters/A | 234 | 42.7 |
| Mon 0.5/109 0.5 | 2 | oz/A | 228 | 41.5 |
| | 1.25 | Liters/A | | |
| Mon 1.0/109 0.5 | 4 | oz/A | 237 | 42.9 |
| | 1.25 | Liters/A | | |

### Example 7 Greenhouse Assay to Determine Activity of Methylobacterium Strains Against Pythium Species

A greenhouse experiment was conducted is to evaluate the ability of Methylobacterium strains to suppress Pythium disease in soybean. Methylobacterium treatments were compared to treatments with metalaxyl at various rates to determine if Methylobacterium strains can provide protection beyond and in addition to the level of protection provided by commonly used agronomic seed treatments. The experiment was designed to allow for growth of plants for in the presence of moderate levels of Pythium. spp. to determine impacts on emergence, % dead seed and root rot incidence and severity.

Pythium inoculants were prepared by mixing 453 g of parboiled rice and 323 mL of distilled water, placing the rice in a vented autoclavable plastic bag, and autoclaving 2×40 minutes with each cycle separated by 24 hours. Rice was inoculated with 3-4-day-old Pythium cultures and incubated for 7-10 days at 230C in the dark. The inoculum was then dried for 2 days.

A 20 ml layer of rice inoculum is placed on soil in cups and additional soil layered on top of the inoculum to position the inoculum approximately 2-3 cm below the seeds to be planted. Water is added to the soil and 10 seeds are planted in each cup. Domes are placed over the cups and the cups are placed in growth chambers with temperature set at 230C, and on a diurnal cycle of 16 h light and 8 h dark. The seeds are incubated for 14 days at 230C. For experiments to assess activity of Methylobacterium strains against a consortium of 4 Pythium species, the seeds are incubated for 14 days at 130C and for an additional 7 days at 230C.

Plants are rated when the first trifoliate is fully emerged and open to determine total plant weight, number of seedlings and number of dead seeds. Disease incidence is evaluated as percent plants with rotted roots and disease severity as percent rotted root tissue for each seedling. Results of the analysis are provided in Tables 31-36 below.

Results from inoculation with Pythium sylvaticum at 230C are shown in Tables 31-32 below.

TABLE 31

| Treatment | Emergence | % Dead Seed |
|---|---|---|
| UTC | 65 | 35 |
| 0.35 Fl. oz/cwt metalaxyl | 82 | 18 |
| NLS0089 | 88 | 12 |
| NLS0109 | 87 | 13 |

NLS0109 and NLS0089 improved emergence and reduced % dead seed better than 7gm/100 kg metalaxyl active ingredient

TABLE 32

| | Mean | Mean Root Rot |
|---|---|---|
| Treatment | Emergence (%) | Severity (%) |
| UTC | 60 | 11 |
| 1.25 Fl. oz/cwt metalaxyl | 78 | 21 |
| 0.75 Fl. oz/cwt metalaxyl | 92 | 18 |
| 0.35 Fl. oz/cwt metalaxyl | 78 | 17 |
| Bacillus subtilis | 83 | 6 |
| NLS0109 | 85 | 5 |

NLS0109 improved emergence and reduced root rot severity better than 7, 15 or 25 gm/100 kg metalaxyl active ingredient

Results from inoculation with Pythium lutarium at 230C are shown in Tables 33-34 below.

TABLE 33

| | Mean | |
|---|---|---|
| Treatment | Emergence (%) | % Dead Seed |
| UTC | 83 | 17 |
| 1.25 Fl. oz/cwt metalaxyl | 93 | 67 |
| 0.75 Fl. oz/cwt metalaxyl | 97 | 3 |
| NLS0109 | 97 | 3 |

NLS0109 improved emergence and reduced percent dead seed better than 25gm/100 kg metalaxyl active ingredient and performed as well as 15 gm/100 kg metalaxyl active ingredient

TABLE 34

| | Mean Root Rot | Mean Root Rot |
|---|---|---|
| Treatment | Incidence (%) | Severity (%) |
| 1.25 Fl. oz/cwt metalaxyl | 93 | 11 |
| 0.75 Fl. oz/cwt metalaxyl | 95 | 10 |
| 0.35 Fl. oz/cwt metalaxyl | 95 | 13 |
| Bacillus subtilis | 93 | 13 |
| NLS0109 | 92 | 9 |

NLS0109 improved emergence and reduced root rot severity better than 7, 15 or 25 gm/100 kg metalaxyl active ingredient

Results from inoculation with a consortium of 4 Pythium species at alternating temperature of 130C and 230C are shown in Tables 35-36 below. The four species, which are all important pathogens of corn and soybean, are P. torulosum, P. oopapillum, P. sylvaticum and P. lutarium.

TABLE 35

| | Mean | Mean Dead |
|---|---|---|
| Treatment | Emergence (%) | Seed (%) |
| UTC | 75 | 25 |
| 0.35 Fl. oz/cwt metalaxyl | 94 | 6 |
| NLS0089 | 100 | 2 |
| NLS0109 | 94 | 2 |
| 1.25 Fl. oz/cwt metalaxyl | 94 | 2 |
| 0.75 Fl. oz/cwt metalaxyl | 100 | 2 |

NLS0109 and NLS0089 improved emergence and NLS0089 reduced % dead seed better than 7gm/100 kg metalaxyl active ingredient.

TABLE 36

| | Root Rot | Root Rot |
|---|---|---|
| Treatment | INC (%) | Severity (%) |
| UTC | 71 | 12 |
| 1.25 Fl. oz/cwt metalaxyl | 94 | 9 |
| 0.75 Fl. oz/cwt metalaxyl | 100 | 12 |
| 0.35 Fl. oz/cwt metalaxyl | 94 | 13 |
| NLS0089 | 94 | 8 |
| NLS0109 | 90 | 7 |

NLS0109 and NLS0089 reduced the incidence and severity of root rot when applied to untreated seed better than 7, 15 or 25 gm/100 kg metalaxyl active ingredient

### Example 8 Analysis of Effect of Methylobacterium Strains on Rhizoctonia and Fusarium Soil Diseases

The effect of Methylobacterium treatment on emergence was evaluated in soy and corn field trials. Improved emergence is evidence of activity of the Methylobacterium treatment against common Fusarium and Rhizoctonia soil pathogens. The trials were conducted as a randomized complete block with four replicates. Standard local agronomic practices for fertilizer application, tillage, row spacing, population, and pest management were used. Standard weed and insect management practices were employed and seed was supplied to cooperators pre-treated with a standard fungicide/insecticide seed treatment. Seed treatment compositions for soybean contained ipconazole, metalaxyl, imidacloprid and a seed coating in addition to the Methylobacterium strains. Seed treatment compositions for corn contained ipconazole, metalaxyl, trifloxystrobin and clothianidin. Stand counts were made at 21 days after first emergence or about 27 days after planting. Effect of Methylobacterium treatment on emergence in corn was evaluated with Methylobacterium provided as a seed treatment or by in furrow application. Results are shown in Tables 37-39 below.

TABLE 37
Soybean Emergence

| Treatment | Stand Count |
|---|---|
| NLS 20/109 | 118432 |
| NLS 109 | 118170 |
| Integral ™ (B. subtilis) | 114456 |
| PPST 2030 | 114431 |
| NLS 64/109 | 112965 |
| UNT | 110376 |
| NLS 17/109 | 109652 |
| NLS 20/109 | 105465 |

Seed treatment with NLS0109 improved soybean stand count in comparison to treatment with Integral™ (B. subtilis, BASF), PPST 2030 (Pioneer Premium Seed Treatment) and untreated control.

TABLE 38
Corn Emergence following Methylobacterium seed treatment

| Treatment | Stand Count |
|---|---|
| NLS 17/109 | 34272 |
| UNT | 34141 |
| NLS 20/109 | 32638 |

TABLE 39
Corn Emergence following Methylobacterium in furrow application

| Treatment | Stand Count |
|---|---|
| NLS 17/109 | 34445 |
| NLS 20/109 | 33302 |
| UNT | 33186 |

Seed treatment with NLS0109 in combination with NLS0017 improved corn emergence in comparison to untreated control.

### REFERENCES

Abanda-Nkpwatt, D., M. Musch, J. Tschiersch, M. Boettner, and W. Schwab. 2006. Molecular interaction between Methylobacterium extorquens and seedlings: growth promotion, methanol consumption, and localization of the methanol emission site. J. Exp. Bot. 57:4025-4032.
Broekaert W F, Terras F R, Cammue B P, Vanderleyden J (1990) An automated quantitative assay for fungal growth inhibition. FEMS Microbiology Letters 69:55-60
Cappellini R A, Peterson J L (1965) Macroconidium formation in submerged cultures by a non-sporulating strain of Gibberella zeae. Mycologia 57:962-966.
Cao, Y-R, Wang, Q., Jin, R-X., Tang, S-K., He, W-X., Lai, H-X, Xu, L-H., and C-L Jiang. 2011. Methylobacterium soli sp. nov. a methanol-utilizing bacterium isolated from the forest soil. Antonie van Leeuwenhoek (2011) 99:629-634.
Cappellini R A, Peterson J L (1965) Macroconidium formation in submerged cultures by a non-sporulating strain of Gibberella zeae. Mycologia 57:962-966
Corpe, W. A., and D. V. Basile. 1982. Methanol-utilizing bacteria associated with green plants. Devel. Industr. Microbiol. 23:483-493.
Corpe, W. A., and S. Rheem. 1989. Ecology of the methylotrophic bacteria on living leaf surfaces. FEMS Microbiol. Ecol. 62:243-250.
Correll J C, Klittich C J R, Leslie J F (1987) Nitrate nonutilizing mutants of Fusarium graminearum and their use in vegetative compatibility tests. Phytopathology 77:1640-1646.
Green, P. N. 2005. Methylobacterium. In Brenner, D. J., N. R. Krieg, and J. T. Staley (eds.). “Bergey's Manual of Systematic Bacteriology. Volume two, The Proteobacteria. Part C, The alpha-, beta-, delta-, and epsilonproteobacteria.” Second edition. Springer, New York. Pages 567-571.
Green, P. N. 2006. Methylobacterium. In Dworkin, M., S. Falkow, E. Rosenberg, K.-H. Schleifer, and E. Stackebrandt (eds.). “The Prokaryotes. A Handbook on the Biology of Bacteria. Volume 5. Proteobacteria: Alpha and Beta Subclasses.” Third edition. Springer, New York. Pages 257-265.
Holland, M. A. 1997. Methylobacterium and plants. Recent. Res. Devel. in Plant Physiol. 1:207-213.
Holland, M. A., and J. C. Polacco. 1994. PPFMs and other covert contaminants: Is there more to plant physiology than just plant? Annu. Rev. Plant Physiol. Plant Mol. Biol. 45:197-209.
Kutschera, U. 2007. Plant-associated methylobacteria as co-evolved phytosymbionts. A hypothesis. Plant Signal Behav. 2:74-78
Lidstrom, M. E. 2006. Aerobic methylotrophic prokaryotes. In Dworkin, M., S. Falkow, E.
Rosenberg, K.-H. Schleifer, and E. Stackebrandt (eds.). “The Prokaryotes. A Handbook on the Biology of Bacteria. Volume 2. Ecophysiology and biochemistry.” Third edition. Springer, New York. Pages 618-634.
Madhaiyan, M., S. Poonguzhali, H.S. Lee, K. Hari, S. P. Sundaram, and T. M. Sa. 2005. Pink-pigmented facultative methylotrophic bacteria accelerate germination, growth and yield of sugarcane clone Co86032 (Saccharum officinarum L.) Biol. Fertil. Soils 41:350-358.
Madhaiyan, M., S. Poonguzhali, M. Senthilkumar, S. Seshadri, H. Chung, J. Yang, S. Sundaram, and T. Sa. 2004. Growth promotion and induction of systemic resistance in rice cultivar C0-47 (Oryza sativa L.) by Methylobacterium spp. Bot. Bull. Acad. Sin. 45:315-324.
Madhaiyan, M., S. Poonguzhali, and T. Sa. 2007. Influence of plant species and environmental conditions on epiphytic and endophytic pink-pigmented facultative methylotrophic bacterial populations associated with field-grown rice cultivars. J Microbiol Biotechnol. 2007 Oct;17(10):1645-54.
Ringler, G. A. 1995. Reaction of soybean to inoculation with Fuusarium solani. MS thesis. Univ. of Illinois, Urbana-Champaign.
Roy, K. W., J. C. Rupe, D. E. Hershman, and T. S. Abney. 19997. Sudden death syndrome of soybean, Plant Dis. 81:1100-1111.
Rupe, J. C, E. E. Gbur, and D. M. Marx. 1991. Cultivar responses to sudden death syndrome of soybean. Plant Dis. 75:47-50.
Spelbrink R G, Dilmac N, Allen A, Smith T J, Shah D M, et al. (2004) Differential antifungal and calcium channel-blocking activity among structurally related plant defensins. Plant Physiol 135:2055-2067.
Stanier, R.Y., N.J. Palleroni, and M. Doudoroff. 1966. The aerobic pseudomonads: A taxonomic study. J. Gen. Microbiol. 43:159-271.
Sy, A., Giraud, E., Jourand, P., Garcia, N., Willems, A., De Lajudie, P., Prin, Y., Neyra, M., Gillis, M., Boivin-Masson,C., and Dreyfus, B. 2001. Methylotrophic Methylobacterium Bacteria Nodulate and Fix Nitrogen in Symbiosis with Legumes. Jour. Bacteriol. 183 (1): 214-220, Sy, A., A .C. J. Timmers, C. Knief, and J. A. Vorholt. 2005. Methylotrophic metabolism is advantageous for Methylobacterium extorquens during colonization of Medicago truncatula under competitive conditions. Appl. Environ. Microbiol. 71:7245-7252.
Vogel, H. J., and D. M. Bonner. 1956. Acetylornithinase of Escherichia coli: Partial purification and some properties. J. Biol. Chem. 218:97-106.
Vogel, H. J. 1956. A convenient growth medium for Neurospora (Medium N). Microbial Genet Bull 13:42-43
Whittenbury, R., S. L. Davies, and J. F. Wilkinson. 1970. Enrichment, isolation and some properties of methane-utilizing bacteria. J. Gen. Microbiol. 61:205-218.
Wrather, J. A. 2010. Soybean disease loss estimates for the United States 1996-2010. Missouri Agric. Res. Sta., Delta Research Center. At the http internet site “aes.missouri.edu/delta/research/soyloss.htm”

The inclusion of various references herein is not to be construed as any admission by the Applicant that the references constitute prior art. Applicants expressly reserve their right to challenge any allegations of unpatentability of inventions disclosed herein over the references included herein.

Having illustrated and described the principles of the present disclosure, it should be apparent to persons skilled in the art that the disclosure can be modified in arrangement and detail without departing from such principles.

Although the materials and methods of this disclosure have been described in terms of various embodiments and illustrative examples, it will be apparent to those of skill in the art that variations can be applied to the materials and methods described herein without departing from the concept, spirit and scope of the disclosure. All such similar substitutes and modifications apparent to those skilled in the art are deemed to be within the spirit, scope and concept of the disclosure as defined by the appended claims or otherwise disclosed herein.

1. A composition comprising a mono- or co-culture of Methylobacterium that inhibits growth of a plant pathogenic fungus and an agriculturally acceptable excipient and/or an agriculturally acceptable adjuvant, wherein:
(i) the Methylobacterium has a 16S nucleic acid sequence having at least 97%, 98%, 99%, 99.5%, or 100% sequence identity to SEQ ID NO: 8;
(ii) the Methylobacterium has a sequence of any one of SEQ ID NOS: 9-11; or,
(iii) the Methylobacterium is NLS0109 or a derivative thereof.

2. The composition of claim 1, wherein said plant pathogenic fungus is a Blumeria sp., a Cercospora sp., a Cochliobolus sp., a Colletotrichum sp., a Diplodia sp., an Exserohilum sp., a Fusarium sp., Gaeumanomyces sp., Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. or a Verticillium sp.

3. The composition of claim 2, wherein said plant pathogenic fungus is a Fusarium sp., a Rhizoctonia sp., a Colletotrichum sp., a Cercospora sp., a Septoria sp., a Pythium sp., a Puccinia sp. or a Sclerotinia sp.

4. The composition of claim 3, wherein said Fusarium sp. is Fusarium graminearum, Fusarium verticillioides, Fusarium oxysporum, or Fusarium solani; wherein said Rhizoctonia sp. is Rhizoctonia solani or Rhizoctonia cerealis; wherein said Colletotrichum sp. is Colletotrichum graminicola; wherein said Cercospora sp. is Cercospora zeae-maydis, Cercospora sojina, or Cercospora kikuchii; wherein said Septoria sp. is Septoria glycines or Septoria tritici; wherein said Pythium sp. is Pythium sylvaticum, Pythium aphanidermatum, Pythium ultimum, Pythium torulosum, Pythium lutarium or Pythium oopapillum; wherein said Puccinia sp. is Puccinia sorghi; or wherein said Sclerotinia sp. is Sclerotinia sclerotiorum or Sclerotinia homoeocarpa.

5. The composition of claim 1, wherein the composition further comprises Methylobacterium strain NLS0017, NLS0020, NLS0089, a derivative thereof, or a Methylobacterium strain related thereto.

6. The composition of claim 5, wherein said Methylobacterium strain related to NLS0017, NLS0020, or NLS0089, has a sequence of any one of SEQ ID NOS: 21-24, 37-39 or 49-51.

7. The composition of claim 1, wherein the Methylobacterium sp. that inhibits growth of a plant pathogenic fungus is NLS0109 or a derivative thereof.

8. The composition of any one of claims 1-7, wherein the composition further comprises a fungicide.

9. The composition of claim 8 wherein said fungicide is a strobilurin, a triazole, a phenylamide, thiazole-carboxamide, or piperidinyl thiazole isoxazoline fungicide.

10. The composition of claim 9, wherein the strobilurin fungicide is azoxystrobin, pyraclostrobin, fluoxystrobin, or trifloxystrobin.

11. The composition of claim 9 wherein the fungicide is a phenylamide, thiazole-carboxamide, or piperidinyl thiazole isoxazoline fungicide.

12. The composition of claim 11, wherein the fungicide is metalaxyl, mefenoxam, ethaboxam or oxathiapiprolin.

13. The composition of claim 9, wherein the triazole fungicide is tebuconazole.

14. A method for controlling a plant pathogenic fungus that comprises applying a first composition comprising the composition of any one of claims 1-13 to a plant, a plant part, to soil where a plant is grown, or any combination thereof in an amount that provides for inhibition of infection by said plant pathogenic fungus in said plant, plant part, or a plant obtained therefrom relative to infection of a control plant, plant part, or plant that had not received an application of said composition or been grown in soil treated with the composition.

15. The method of claim 14, wherein application of said composition provides for at least a 40%, 50%, 75%, at least 85%, or at least 95% inhibition of a plant pathogenic fungal infection in said plant, plant part, or a plant derived therefrom relative to infection of the control plant, plant part, or plant.

16. The method of claim 15, wherein said plant part is selected from the group consisting of a leaf, a stem, a fruit, a flower, a root, a tuber, and a seed.

17. The method of claim 14, wherein mycotoxin levels in said plant part are reduced by at least 50%, at least 75%, at least 85%, or at least 95% relative to a plant part obtained from the control plant, plant part, or plant obtained therefrom.

18. The method of claim 14, wherein said method further comprises obtaining a processed food or feed composition from said plant or plant part.

19. The method of claim 18, wherein mycotoxin levels in said processed food or feed composition are reduced by at least 50%, at least 75%, at least 85%, or at least 95% relative to a processed food or feed composition obtained from the control plant, plant part, or plant obtained therefrom.

20. The method of claim 14, wherein the plant or plant part is a cereal plant or plant part.

21. The method of claim 20, wherein the cereal plant or plant part is selected from the group consisting of a rice, wheat, corn, barley, millet, sorghum, oat, and rye plant or plant part.

22. The method of claim 14, wherein the Methylobacterium is NLS0109 or a derivative thereof, or a Methylobacterium strains comprising any one of SEQ ID NO:9-11.

23. The method of claim 22, further comprising applying a second Methylobacterium to a plant, a plant part, to soil where a plant is grown, or any combination thereof, wherein said second Methylobacterium is NLS0017, NLS0020, NLS0089, or a derivative thereof, or a Methylobacterium sp. comprising any one of SEQ ID NOS: 21-24, 37-39 or 49-51.

24. The method of claim 23, wherein the second Methylobacterium is applied to the soil in furrow with the plant part, on a seed from which the plant is grown, or to the plant foliage.

25. The method of claim 23, wherein Methylobacterium NLS0109 and said second Methylobacterium are applied simultaneously.

26. The method of any one of claims 14-25, wherein said method further comprises applying a fungicide in a composition comprising said Methylobacterium or following application of said Methylobacterium.

27. The method of claim 26, wherein the fungicide is azoxystrobin, pyraclostrobin, fluoxystrobin, or trifloxystrobin.

28. The method of claim 27, wherein the strobilurin fungicide is pyraclostrobin.

29. The method of claim 26, wherein the fungicide is active against Pythium sp.

30. The composition of claim 26, wherein the fungicide is metalaxyl, mefenoxam, ethaboxam or oxathiapiprolin.

31. The composition of claim 30, wherein the fungicide is metalaxyl.

32. A method of making the composition of any one of claims 1-13 that comprises combining a Methylobacterium that inhibits growth of a plant pathogenic fungus with an agriculturally acceptable excipient and/or with an agriculturally acceptable adjuvant.

33. The method of claim 32, wherein the composition is an emulsion.

34. The method of claim 33, wherein the Methylobacterium is NLS0109 and the plant pathogenic fungus is a Fusarium sp., a Rhizoctonia sp., a Colletotrichum sp., a Cercospora sp., a Septoria sp., a Pythium sp., a Puccinia sp. or a Sclerotinia sp.

35. An isolated Methylobacterium that inhibits growth of a plant pathogenic fungus, wherein the Methylobacterium has a 16S RNA having at least 97%, 98%, 99%, 99.5%, or 100% sequence identity to SEQ ID NO:8 or has a sequence of any one of SEQ ID NOS: 9-11.

36. The Methylobacterium of claim 35, wherein said Methylobacterium is NLS0109.

37. The Methylobacterium of claim 35, wherein said plant pathogenic fungus is selected from the group consisting of a Blumeria sp., a Cercospora sp., a Cochliobolus sp., a Colletotrichum sp., a Diplodia sp., an Exserohilum sp., a Fusarium sp., a Gaeumanomyces sp., a Macrophomina sp., a Magnaporthe sp., a Microdochium sp., a Peronospora sp., a Phakopsora sp., a Phialophora sp., a Phoma sp., a Phymatotrichum sp., a Phytophthora sp., a Pyrenophora sp., a Pyricularia sp, a Pythium sp., a Rhizoctonia sp., a Sclerophthora, a Sclerospora sp., a Sclerotium sp., a Sclerotinia sp., a Septoria sp., a Stagonospora sp., a Stenocarpella sp. and a Verticillium sp.

38. The Methylobacterium of claim 37, wherein said plant pathogenic fungus is a Fusarium sp.

39. The Methylobacterium of claim 38, wherein said Fusarium sp. is selected from the group consisting of Fusarium graminearum, Fusarium verticillioides, Fusarium oxysporum, and Fusarium solani.

40. A plant that is at least partially coated with the composition of any one of claims 1-13.

41. The plant of claim 40, wherein the plant is a cereal plant.

42. The plant of claim 41, wherein the cereal plant is selected from the group consisting of a rice, wheat, corn, barley, millet, sorghum, oat, and rye plant part.

43. The plant of claim 42, wherein the Methylobacterium was obtained from a plant genus, plant species, plant sub-species, or plant cultivar that is distinct from the genus, species, sub-species, or cultivar of the plant.

44. An isolated plant part that is at least partially coated with the composition of any one of claims 1-13.

45. The plant part of claim 44, wherein the plant part is a cereal plant part.

46. The plant part of claim 45, wherein the cereal plant part is selected from the group consisting of a rice, wheat, corn, barley, millet, sorghum, oat, and rye plant part.

47. The plant part of claim 40, wherein the Methylobacterium was obtained from a plant genus, plant species, plant sub-species, or plant cultivar that is distinct from the genus, species, sub-species, or cultivar of the plant part.

48. A processed plant product that comprises a detectable amount of the Methylobacterium of any of the compositions of any one of claims 1-13.

49. The processed plant product of claim 48, wherein the Methylobacterium that is detected was obtained from a plant genus, plant species, plant sub-species, or plant cultivar that is distinct from the genus, species, sub-species, or cultivar used to obtain the processed plant product.

50. A method to detect the presence of Methylobacterium strain NL0109, NLS0020, NLS0017, or NLS0089 in a sample, wherein said method comprises detecting the presence in the sample of a nucleic acid comprising or located within SEQ ID NO:9-11, 21-24, 37-39 or 49-51.

51. The method of claim 50, wherein the detecting of the sequence comprises a polymerase chain reaction, branched DNA, ligase chain reaction, transcription mediated amplification (TMA), nucleic acid sequence-based amplification (NASBA), nanopore-, mass spectroscopy, hybridization, or direct sequencing based method, or any combination thereof.

52. The method of claim 51, said detection comprises the steps of (i) contacting the sample with a DNA primer pair, wherein said primer pair comprises forward and reverse primers for amplification of a DNA fragment comprising or located within SEQ ID NO:9-11, 21-24, 37-39 or 49-51, thereby generating a DNA fragment,
(ii) contacting said DNA fragment with a probe specific for the presence of said DNA fragment, and
(iii) comparing the results of said contacting with positive and negative controls to determine the presence of SEQ ID NO:9-11, 21-24, 37-39 or 49-51 in said sample.

53. The method of claim 50 wherein said sample is a plant material that was treated with one or more of Methylobacterium strains selected from NL0109, NLS0020, NLS0017, or NLS0089.

54. The method of claim 52 wherein said plant material is leaves, roots or seeds.

55. The method of claim 52 wherein the plant material is a processed plant product from a plant treated with one or more Methylobacterium strains selected from NLS0109, NLS0020, NLS0017, or NLS0089.

56. The method of claim 50 wherein said sample is a soil sample.


SEQUENCE LISTING (60 sequences)

SEQ ID NO: 1 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggggatttgctccagatcgctccttcgcctcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcgcgccaccgaagtggaccccaaatctctctgggtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgctactgcggtgcaagcaccccaacagctggcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagccctagtttcccagggttattccgaaccagaaggcacgttcccacgcgttactcacccgtccgccgctgaccccgaaaggcccgctcgacttgcatgtgttaagcctgccgccagcgttcgctctgagccaggatcaaactctc

SEQ ID NO: 2 - DNA, 1471 bp, Methylobacterium sp.
  source 1..1471: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggggatttgctccagatcgctccttcgcgtcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcgcgccaccgaagtggaccccaaatctctctgggtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgctactgcggtgcaagcaccccaacagctggcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagccctagtttcccagggttattccgaaccagaaggcacgttcccacgcgttactcacccgtccgccgctgaccccgaagggcccgctcgacttgcatgtgttaagcctgccgccagcgttcgctctgagccaggatcaaactctc

SEQ ID NO: 3 - DNA, 1472 bp, Methylobacterium sp.
  source 1..1472: mol_type = genomic DNA
gagtttgatcctggctcagagcgaacgctggcggcaggcttaacacatgcaagtcgaacgggcttcttcggaagtcagtggcagacgggtgagtaacacgtgggaacgtgcccttcggttcggaataactcagggaaacttgagctaataccggatacgcccttatggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggtctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaaccgcgaggttgagcaaatccccaaaagccgtctcagttcggattgcactctgcaactcgggtgcatgaaggcggaatcgctagtaatcgtggatcagcacgccacggtgaatacgttcccgggccttgtacacaccgcccgtcacaccatgggagttggtcttacccgacggcgctgcgccaaccgcaagggggcaggcgaccacggtagggtcagcgactggggtgaagtcgtaacaaggtagccgtaggggaacctgcggctggatcacct

SEQ ID NO: 4 - DNA, 1473 bp, Methylobacterium sp.
  source 1..1473: mol_type = genomic DNA
ggtgatccagccgcaggttcccctacggctaccttgttacgacttcaccccagtcgctgaccctaccgtggtcgcctgcctccttgcggttggcgcagcgccgtcgggtaagaccaactcccatggtgtgacgggcggtgtgtacaaggcccgggaacgtattcaccgtggcgtgctgatccacgattactagcgattccgccttcatgcacccgagttgcagagtgcaatccgaactgagacggtttttggggatttgctccacctcgcggcttcgcgtcccactgtcaccgccattgtagcacgtgtgtagcccatcccgtaagggccatgaggacttgacgtcatccacaccttcctcgcggcttatcaccggcagtctccctagagtgcccaactgaatgatggcaactaaggacgtgggttgcgctcgttgcgggacttaacccaacatctcacgacacgagctgacgacagccatgcagcacctgtgtgcacgcctccgaagaggatccccgatctctcgaggtaacatgccatgtcaagggatggtaaggttctgcgcgttgcttcgaattaaaccacatgctccaccgcttgtgcgggcccccgtcaattcctttgagttttaatcttgcgaccgtactccccaggcggaatgcttaatgcgttagcggcgccactgacctgcaagcaggccaacggctggcattcatcgtttacggcgtggactaccagggtatctaatcctgtttgctccccacgctttcgcgcctcagcgtcagaaccggaccagacagccgccttcgccactggtgttcttgcgaatatctacgaatttcacctctacactcgcagttccgctgtcctcttccggtctcaagccaaccagtatcgaaggcaattctgtggttgagccacaggctttcacccccgacttaatcggccgcctacgcgccctttacgcccagtgattccgagcaacgctagcccccttcgtattaccgcggctgctggcacgaagttagccggggcttattcttccggtaccgtcattatcgtcccggacaaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaacaagctaatcagacgcgggccgatccttcggcagtaaacctttccccaaaagggcgtatccggtattagctcaagtttccctgagttattccgaaccgaagggtacgttcccacgtgttactcacccgtctgccactgacacccgaaggtgcccgttcgacttgcatgtgttaagcctgccgccagcgttcgctctgagccaggatcaaactctc

SEQ ID NO: 5 - DNA, 1484 bp, Methylobacterium sp.
  source 1..1484: mol_type = genomic DNA
ggtgatccagccgcaggttcccctacggctaccttgttacgacttcaccccagtcgctgaccctaccgtggtcgcctgcctccagtcgagcaagctcgatttggttggcgcagcgccgtcgggtaagaccaactcccatggtgtgacgggcggtgtgtacaaggcccgggaacgtattcaccgtggcatgctgatccacgattactagcgattccgccttcatgcacgcgagttgcagcgtgcaatccgaactgagacggcttttggagattggctccgggtcaccccttcgcgtcccactgtcaccgccattgtagcacgtgtgtagcccatcccgtaagggccatgaggacttgacgtcatccacaccttcctcgcggcttatcaccggcagtctccctagagtgcccaaccaaatgatggcaactaaggacgtgggttgcgctcgttgcgggacttaacccaacatctcacgacacgagctgacgacagccatgcagcacctgtgtgcgcgcccccgaaggggacctggaatctctcccagtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgcttaatgcgttagctgcgctactgcggtgcatgcaccccaacagctagcattcatcgtttacggcgtggactaccagggtatctaatcctgtttgctccccacgctttcgcgcctcagcgtcagtaatggtccagttggccgccttcgccaccggtgttcttgcgaatatctacgaatttcacctctacactcgcagttccaccaacctctaccatactcaagcgtcccagtatcgaaggccattctgtggttgagccacaggctttcacccccgacttaaaacgccgcctacgcgccctttacgcccagtgattccgagcaacgctagcccccttcgtattaccgcggctgctggcacgaagttagccggggcttattcctccggtaccgtcattatcgtcccggagaaaagagctttacaaccctaaggccgtcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaacaagctaatcagacgcgggccgatcctccggcagtaaacctttctgccaaagcacgtatccggtattagccctagtttcccagggttatcccagaccggagggcacgttcccacgtgttactcacccgtctgccactcaccttgcggtgcgttcgacttgcatgtgttaagcctgccgccagcgttcgctctgagccaggatcaaactctc

SEQ ID NO: 6 - DNA, 1484 bp, Methylobacterium sp.
  source 1..1484: mol_type = genomic DNA
gagtttgatcctggctcagagcgaacgctggcggcaggcttaacacatgcaagtcgaacgcaccgcaaggtgagtggcagacgggtgagtaacacgtgggaacgtgccctccggtctgggataaccctgggaaactagggctaataccggatacgtgctttggcagaaaggtttactgccggaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgacggccttagggttgtaaagctcttttctccgggacgataatgacggtaccggaggaataagccccggctaacttcgtgccagcagccgcggtaatacgaagggggctagcgttgctcggaatcactgggcgtaaagggcgcgtaggcggcgttttaagtcgggggtgaaagcctgtggctcaaccacagaatggccttcgatactgggacgcttgagtatggtagaggttggtggaactgcgagtgtagaggtgaaattcgtagatattcgcaagaacaccggtggcgaaggcggccaactggaccattactgacgctgaggcgcgaaagcgtggggagcaaacaggattagataccctggtagtccacgccgtaaacgatgaatgctagctgttggggtgcatgcaccgcagtagcgcagctaacgcattaagcattccgcctggggagtacggtcgcaagattaaaactcaaaggaattgacgggggcccgcacaagcggtggagcatgtggtttaattcgaagcaacgcgcagaaccttaccatcctttgacatggcgtgttactgggagagattccaggtccccttcgggggcgcgcacacaggtgctgcatggctgtcgtcagctcgtgtcgtgagatgttgggttaagtcccgcaacgagcgcaacccacgtccttagttgccatcatttggttgggcactctagggagactgccggtgataagccgcgaggaaggtgtggatgacgtcaagtcctcatggcccttacgggatgggctacacacgtgctacaatggcggtgacagtgggacgcgaaggggtgacccggagccaatctccaaaagccgtctcagttcggattgcacgctgcaactcgcgtgcatgaaggcggaatcgctagtaatcgtggatcagcatgccacggtgaatacgttcccgggccttgtacacaccgcccgtcacaccatgggagttggtcttacccgacggcgctgcgccaaccaaatcgagcttgctcgactggaggcaggcgaccacggtagggtcagcgactggggtgaagtcgtaacaaggtagccgtaggggaacctgcggctggatcacctc

SEQ ID NO: 7 - DNA, 1472 bp, Methylobacterium sp.
  source 1..1472: mol_type = genomic DNA
gagtttgatcctggctcagagcgaacgctggcggcaggcttaacacatgcaagtcgaacgggcttcttcggaagtcagtggcagacgggtgagtaacacgtgggaacgtgcccttcggttcggaataactcagggaaacttgagctaataccggatacgcccttacggggaaaggtttactgccgaaggatcggcccgcgtctgattagcttgttggtggggtaacggcctaccaaggcgacgatcagtagctggtctgagaggatgatcagccacactgggactgagacacggcccagactcctacgggaggcagcagtggggaatattggacaatgggcgcaagcctgatccagccatgccgcgtgagtgatgaaggccttagggttgtaaagctcttttgtccgggacgataatgacggtaccggaagaataagccccggctaacttcgtgccagcagccgcggtaatacgaagggggctagcgttgctcggaatcactgggcgtaaagggcgcgtaggcggccgattaagtcgggggtgaaagcctgtggctcaaccacagaattgccttcgatactggttggcttgagaccggaagaggacagcggaactgcgagtgtagaggtgaaattcgtagatattcgcaagaacaccagtggcgaaggcggctgtctggtccggttctgacgctgaggcgcgaaagcgtggggagcaaacaggattagataccctggtagtccacgccgtaaacgatgaatgccagccgttggtctgcttgcaggtcagtggcgccgctaacgcattaagcattccgcctggggagtacggtcgcaagattaaaactcaaaggaattgacgggggcccgcacaagcggtggagcatgtggtttaattcgaagcaacgcgcagaaccttaccatcccttgacatggcatgttacctcgagagatcggggatcctcttcggaggcgtgcacacaggtgctgcatggctgtcgtcagctcgtgtcgtgagatgttgggttaagtcccgcaacgagcgcaacccacgtccttagttgccatcattcagttgggcactctagggagactgccggtgataagccgcgaggaaggtgtggatgacgtcaagtcctcatggcccttacgggatgggctacacacgtgctacaatggcggtgacagtgggacgcgaaaccgcgaggttgagcaaatccccaaaagccgtctcagttcggattgcactctgcaactcgggtgcatgaaggcggaatcgctagtaatcgtggatcagcacgccacggtgaatacgttcccgggccttgtacacaccgcccgtcacaccatgggagttggtcttacccgacggcgctgcgccaaccgcaagggggcaggcgaccacggtagggtcagcgactggggtgaagtcgtaacaaggtagccgtaggggaacctgcggctggatcacct

SEQ ID NO: 8 - DNA, 1473 bp, Methylobacterium sp.
  source 1..1473: mol_type = genomic DNA
ggtgatccagccgcaggttcccctacggctaccttgttacgacttcaccccagtcgctgaccctaccgtggtcgcctgctccccttgcgggtcggcgcagcgccgtcgggtaagaccaactcccatggtgtgacgggcggtgtgtacaaggcccgggaacgtattcaccgtggcatgctgatccacgattactagcgattccgccttcatgcactcgagttgcagagtgcaatccgaactgagacggcttttggagatttgcttgccctcgcgggttcgcgtcccactgtcaccgccattgtagcacgtgtgtagcccatcccgtaagggccatgaggacttgacgtcatccacaccttcctcgcggcttatcaccggcagtctccccagagtgcccaactgaatgatggcaactgaggacgtgggttgcgctcgttgcgggacttaacccaacatctcacgacacgagctgacgacagccatgcagcacctgtgtgcgcgctcccgaaggagaccgtggatctctccacgtaacacgccatgtcaaaggatggtaaggttctgcgcgttgcttcgaattaaaccacatgctccaccgcttgtgcgggcccccgtcaattcctttgagttttaatcttgcgaccgtactccccaggcggaatgctcaaagcgttagctgcgccactgagaggcaagccccccaacggctggcattcatcgtttacggcgtggactaccagggtatctaatcctgtttgctccccacgctttcgcgcctcagcgtcagtgtcggaccagttggccgccttcgccaccggtgttcttgcgaatatctacgaatttcacctctacactcgcagttccaccaacctcttccgaactcaagtctcccagtatcgaaggcaattctgtggttgagccacaggctttcacccccgacttaaaagaccgcctacgcgccctttacgcccagtgattccgagcaacgctagcccccttcgtattaccgcggctgctggcacgaagttagccggggcttattcctccggtaccgtcattatcgtcccggataaaagagctttacaaccctaaggccttcatcactcacgcggcatggctggatcaggcttgcgcccattgtccaatattccccactgctgcctcccgtaggagtctgggccgtgtctcagtcccagtgtggctgatcatcctctcagaccagctactgatcgtcgccttggtaggccgttaccccaccaactagctaatcagacgcgggccgatcttccggcagtaaacctttccccaaaagggcgtatccggtattagctcaagtttccctgagttattccgaaccagaaggcacgttcccacgcgttactcacccgtccgccgctgacaccgaagtgcccgctcgacttgcatgtgttaagcctgccgccagcgttcgctctgagccaggatcaaactctc

SEQ ID NO: 9 - DNA, 300 bp, Methylobacterium sp.
  source 1..300: mol_type = genomic DNA
acggtcaccccacggactgggcgagtacctcaccggtgttctatcataacgccgagttagttttcgaccgtcccttatgcgatgtaccaccggtgtcggcagccgatttcgtcccaccgggagctggcgttccggttcagaccaccatcatcggtcacgatgtctggattggacacggggccttcatctcccccggcgtgactataggaaacggcgcgatcgtcggggcccaggcggtcgtcacaagagatgtcccaccctatgcggtagttgctggcgtccccgcgaccgtacgacgat

SEQ ID NO: 10 - DNA, 300 bp, Methylobacterium sp.
  source 1..300: mol_type = genomic DNA
ccaataaaagcgttggccgcctgggcaacccgatccgagcctaagactcaaagcgcaagcgaacacttggtagagacagcccgccgactacggcgttccagcactctccggctttgatcggataggcattggtcaaggtgccggtggtgatgacctcgcccgccgcaagcggcgaattactcggatcagcggccagcacctcgaccaagtgtcggagcgcgaccaaagggccacgttcgaggacgtttgaggcgcgaccagtctcgatagtctcatcgtcgcggcgaagctgcacctcga

SEQ ID NO: 11 - DNA, 300 bp, Methylobacterium sp.
  source 1..300: mol_type = genomic DNA
cgatggcaccgacctgccatgcctctgccgtccgcgccagaatggtaaagaggacgaagggggtaaggatcgtcgctgcagtgttgagcagcgaccagagaagggggccgaacatcggcatcaaacctcgattgccactcggacgcgaagcgcgtcttgaaggagggatggaagcgaaacggccgcagagtaaccgccgacgaaagattgcacccctcatcgagcaggatcggaggtgaaggcaagcgtgggttattggtaagtgcaaaaaatataatggtagcgtcagatctagcgttc

SEQ ID NO: 12 - DNA, 23 bp, synthetic construct
  source 1..23: mol_type = other DNA
cctcaccggtgttctatcataac

SEQ ID NO: 13 - DNA, 20 bp, synthetic construct
  source 1..20: mol_type = other DNA
ccgatgatggtggtctgaac

SEQ ID NO: 14 - DNA, 21 bp, synthetic construct
  source 1..21: mol_type = other DNA
cgtcccttatgcgatgtacca

SEQ ID NO: 15 - DNA, 22 bp, synthetic construct
  source 1..22: mol_type = other DNA
gatccgagcctaagactcaaag

SEQ ID NO: 16 - DNA, 21 bp, synthetic construct
  source 1..21: mol_type = other DNA
gaccaatgcctatccgatcaa

SEQ ID NO: 17 - DNA, 20 bp, synthetic construct
  source 1..20: mol_type = other DNA
aacacttggtagagacagcc

SEQ ID NO: 18 - DNA, 21 bp, synthetic construct
  source 1..21: mol_type = other DNA
aaggagggatggaagcgaaac

SEQ ID NO: 19 - DNA, 19 bp, synthetic construct
  source 1..19: mol_type = other DNA
ataacccacgcttgccttc

SEQ ID NO: 20 - DNA, 21 bp, synthetic construct
  source 1..21: mol_type = other DNA
cgcagagtaaccgccgacgaa

SEQ ID NO: 21 - DNA, 300 bp, Methylobacterium sp.
  source 1..300: mol_type = genomic DNA
gcccttctgtcaggcgatattgtataatggcgttgccccaatagaagcagccattcgtgcgagggcagcagcgacgctaggtcgaaagagcatcctaatctcgatcaagatgcgactgagatttctgatgaaaatatctagacacaagcaaagctggtgaaattacaacgatcatggcgacaattgcggccaattcggccggaacttgaaggaacataaaaatgaatattacaaatataccgcaaagcatgtagagttgctacaccaagggtcgggacgtccaaaaaaactcactgagga

SEQ ID NO: 22 - DNA, 300 bp, Methylobacterium sp.
  source 1..300: mol_type = genomic DNA
ggaacataaaaatgaatattacaaatataccgcaaagcatgtagagttgctacaccaagggtcgggacgtccaaaaaaactcactgaggaagtcgactggaagcacgaggcgccccccccaggagcggggcgaccggcaagggggcccgcaattgtcgccatgatcgaccagcttaggtaggatcctctttcgacctaacgaatggctgcttctattggggcaacgccattatacaatatcgcctgaccatctggaacgcggcccggtccaccggcaggttggcgacgacagcgtcggag

SEQ ID NO: 23 - DNA, 300 bp, Methylobacterium sp.
  source 1..300: mol_type = genomic DNA
cggcgtcgaccagccgggcgaactgcttgggcatgctctcccgcgacgccggccacagccgcgtccccgtccctccgcacaggatcatcgggtggatttgaaaggcaaaacgggacatcaggataggccgctcaggcgttggcgctgaggcgcttgatgtcggcgtcgaccatctcggtgatcagcgcctcgaggctggtctcggcctcccagccgaaggtcgccttggccttggcggggttgcccagcagcacctcgacctctgccggccggaacagcgccgggtcgacgatcaggtgg

SEQ ID NO: 24 - DNA, 300 bp, Methylobacterium sp.
  source 1..300: mol_type = genomic DNA
ctggacatgcgcccaccccggccaagtccgaccgcaccggcaaccgctcctgtagtcgtcgtcatcgttctcacccctgaggcggagaccgtccgctaacggggtgtctcaagcaaccgtggggcggaggaacacgcacgtagtcgcgtttcaaggttcgcacgaacgcctcggccatgccgttgctctgcgggctctccagcggcgtcgtttttggcaccaaaccaaggtcgcgggcgaagcggcgcgtgtcgcggggactgtcaggaatttcgtgtgggggcggccatagtggatccg

SEQ ID NO: 25 - DNA, 18 bp, synthetic construct
  source 1..18: mol_type = other DNA
cacaagcaaagctggtga

SEQ ID NO: 26 - DNA, 19 bp, synthetic construct
  source 1..19: mol_type = other DNA
aagatatgctttgcgggta

SEQ ID NO: 27 - DNA, 18 bp, synthetic construct
  source 1..18: mol_type = other DNA
cgatcatggcgacaattg

SEQ ID NO: 28 - DNA, 20 bp, synthetic construct
  source 1..20: mol_type = other DNA
caatatcgcctgacagaagg

SEQ ID NO: 29 - DNA, 20 bp, synthetic construct
  source 1..20: mol_type = other DNA
cacttcaacaaaggcgatca

SEQ ID NO: 30 - DNA, 14 bp, synthetic construct
  source 1..14: mol_type = other DNA
ttggcgacgacagc

SEQ ID NO: 31 - DNA, 19 bp, synthetic construct
  source 1..19: mol_type = other DNA
actgcttgggcatgctctc

SEQ ID NO: 32 - DNA, 20 bp, synthetic construct
  source 1..20: mol_type = other DNA
cctatcctgatgtcccgttt

SEQ ID NO: 33 - DNA, 20 bp, synthetic construct
  source 1..20: mol_type = other DNA
aggatcatcgggtggatttg

SEQ ID NO: 34 - DNA, 18 bp, synthetic construct
  source 1..18: mol_type = other DNA
aggaacacgcacgtagtc

SEQ ID NO: 35 - DNA, 18 bp, synthetic construct
  source 1..18: mol_type = other DNA
ccacacgaaattcctgac

SEQ ID NO: 36 - DNA, 14 bp, synthetic construct
  source 1..14: mol_type = other DNA
tggcaccaaaccaa

SEQ ID NO: 37 - DNA, 300 bp, Methylobacterium sp.
  source 1..300: mol_type = genomic DNA
gcaaaacgacctaatagttctacagcggcatgcgccaagtcagcgcggtgaacagtatacctgggagcaacttgtcctccgaaacccacataaaacaaattactcctggcagtgcccagtccatcaaaatcgaatacaatatttctcgaggaggcatctgtaatagcctgccaaagcaacaaagctatggcgccgttatgactttcattgcttctggtagacataaaataatatgccgatttgtgatcccaaatgtagaatattgccgcatcaattgcgccaagtttatttcggatcgat

SEQ ID NO: 38 - DNA, 300 bp, Methylobacterium sp.
  source 1..300: mol_type = genomic DNA
ggcgccaacggtatgatcgcatgattttcctgcggcatagcttgcgggaatggcgtatttggcgctctcctcaggaatttctaagggcatacgcaggaactctacagcacttttactggtattttgtagtgacagcggaggaggctggtgctcaaggtaatcgtgatgaagtgatccgggccattcggggcgcgtttctagtctttccaatccgcgccctgtaccacgtattacgccggaccggtctgcgccgcgccgccctcttgaccgccctaaatgtctaagagcgtctaacaaagc

SEQ ID NO: 39 - DNA, 300 bp, Methylobacterium sp.
  source 1..300: mol_type = genomic DNA
gacgatatcgctcatcttcactgcattgaagctggtgccgtactgcatagggatgaaaaagtgatgcggatagacggctgacgggaaagcgcctggtcgatcgaagactttgctgacgaggttgtggtagccccggatataggcatcgaaggccgggacgttgatcccatcctttgccttatcttgactggcgtcgtcgcgtgccgtcagaacgggcacgtcgcaggtcatcgaggccagcaccttgcggaacacctgcgttccgccgttgggattatcgacggcgaacgcggtggccgc

SEQ ID NO: 40 - DNA, 21 bp, synthetic construct
  source 1..21: mol_type = other DNA
gtcctccgaaacccacataaa

SEQ ID NO: 41 - DNA, 23 bp, synthetic construct
  source 1..23: mol_type = other DNA
ctaccagaagcaatgaaagtcat

SEQ ID NO: 42 - DNA, 22 bp, synthetic construct
  source 1..22: mol_type = other DNA
tctgtaatagcctgccaaagca

SEQ ID NO: 43 - DNA, 19 bp, synthetic construct
  source 1..19: mol_type = other DNA
ggctggtgctcaaggtaat

SEQ ID NO: 44 - DNA, 20 bp, synthetic construct
  source 1..20: mol_type = other DNA
acatttagggcggtcaagag

SEQ ID NO: 45 - DNA, 19 bp, synthetic construct
  source 1..19: mol_type = other DNA
atgaagtgatccgggccat

SEQ ID NO: 46 - DNA, 21 bp, synthetic construct
  source 1..21: mol_type = other DNA
ccgtactgcatagggatgaaa

SEQ ID NO: 47 - DNA, 21 bp, synthetic construct
  source 1..21: mol_type = other DNA
taaggcaaaggatgggatcaa

SEQ ID NO: 48 - DNA, 21 bp, synthetic construct
  source 1..21: mol_type = other DNA
ttgctgacgaggttgtggtag

SEQ ID NO: 49 - DNA, 300 bp, Methylobacterium sp.
  source 1..300: mol_type = genomic DNA
ggaaatcggcttcaagtacgacgtcacgccggccatgcaggtcacgggtgcactgttcaatctcgagcgcgacaaccagccgttcccctcgaacgtggagtccggcctcgtccttggcgcaggtcagacacgcacccagggcgcggaaatcggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtct

SEQ ID NO: 50 - DNA, 300 bp, Methylobacterium sp.
  source 1..300: mol_type = genomic DNA
cggcctggccggctatctaaccgattggtggcaggtctttggcggctacgcttataccgaggcacgcgtactctcgccactggaagacgatggagacgtgatcgcagcaggtaatctcgtcggcaacgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgac

SEQ ID NO: 51 - DNA, 300 bp, Methylobacterium sp.
  source 1..300: mol_type = genomic DNA
acgttccgctaaatactttcagtctgttcaacaagttcgatatcaacgagaatttctccgttgctctgggctattactatcaggatgccagctttgcctcctcagacaatgcagtgcgtttgccaagttattcgcggttcgatggcgggttgttctatcgattcgacgagttgacacgcgttcagcttagcgtcgagaacattttcgacaggcgttacatcatcaactccaacaacaacaacaacctcacgcctggcgcgccgagaacagtccgcgtgcaattgatcgctcggttctaaa

SEQ ID NO: 52 - DNA, 20 bp, synthetic construct
  source 1..20: mol_type = other DNA
tttggcggctacgcttatac

SEQ ID NO: 53 - DNA, 20 bp, synthetic construct
  source 1..20: mol_type = other DNA
aacgttgccgacgagattac

SEQ ID NO: 54 - DNA, 22 bp, synthetic construct
  source 1..22: mol_type = other DNA
agacgatggagacgtgatcgca

SEQ ID NO: 55 - DNA, 20 bp, synthetic construct
  source 1..20: mol_type = other DNA
ggcaacgttccgctaaatac

SEQ ID NO: 56 - DNA, 20 bp, synthetic construct
  source 1..20: mol_type = other DNA
aaagctggcatcctgatagt

SEQ ID NO: 57 - DNA, 22 bp, synthetic construct
  source 1..22: mol_type = other DNA
cgagaatttctccgttgctctg

SEQ ID NO: 58 - DNA, 19 bp, synthetic construct
  source 1..19: mol_type = other DNA
cgatggcgggttgttctat

SEQ ID NO: 59 - DNA, 18 bp, synthetic construct
  source 1..18: mol_type = other DNA
aggcgtgaggttgttgtt

SEQ ID NO: 60 - DNA, 22 bp, synthetic construct
  source 1..22: mol_type = other DNA
aggcgttacatcatcaactcca